(19) United States
(12) Patent Application Publication
(10) Pub. No.: US 2024/0189278 A1
(43) Pub. Date: Jun. 13, 2024

(54) NOVEL COMPOUNDS AND METHODS FOR INCREASING KLOTHO GENE EXPRESSION

(71) Applicant: Klotho Therapeutics, Inc., Las Vegas, NV (US)

(72) Inventors: James Plante, Las Vegas, NV (US); William Ramage, Del Mar, CA (US); Michael Bishop, San Diego, CA (US)

(21) Appl. No.: 18/206,076

(22) Filed: Jun. 5, 2023

Related U.S. Application Data
(63) Continuation-in-part of application No. 15/985,154, filed on May 21, 2018, now abandoned.
(63) Continuation-in-part of application No. 18/015,040, filed on Jan. 6, 2023.
(60) Provisional application No. 62/509,818, filed on May 23, 2017.
(60) Provisional application No. 63/049,614, filed on Jul. 8, 2020.

(51) Int. Cl. A61K 31/4045; A61P 9/12

(52) U.S. Cl. CPC A61K 31/4045; A61P 9/12

(57) ABSTRACT

Compositions and methods for the treatment and amelioration of arterial stiffness, hypertension, and/or arterial aging in a subject. In embodiments, the active agents of the compositions provide anti-aging treatments by causing arterial remodeling by decreasing collagen production and increasing elastin production in a subject. In certain embodiments, the active agents can be used to treat a subject having diabetes or a diabetes-related disease or condition, such as but not limited to, Type 1 diabetes mellitus (T1DM), Type 2 diabetes mellitus (T2DM), and hyperinsulenima (pre-diabetes). In certain embodiments, the active agents can be used to treat subjects having hypertension, aortic disease, cardiovascular disease, including heart failure (such as congestive heart failure), kidney disease, osteoporosis, Alzheimer's disease, infertility, and emphysema.

### CROSS-REFERENCE TO RELATED APPLICATIONS

This application is a continuation-in-part of U.S. Application No. 62/509,818, filed on May 23, 2017, claims priority to U.S. Provisional Application No. 62/509,818, filed on May 23, 2017, which is expressly incorporated herein by reference in its entirety.

### STATEMENT REGARDING FEDERALLY SPONSORED RESEARCH OR DEVELOPMENT

One or more aspects or embodiments of this invention was made with government support under Contract Numbers HL105302 and HL102074 from the National Institutes of Health. The government has certain rights in the invention.

### BACKGROUND1

### 1. Technical Field

The present disclosure relates to novel compounds and compositions comprising the same and to methods of manufacturing and using the same, particularly for increasing Klotho gene expression.

### 2. Related Technology

Millions of people suffer from heart attack and stroke each year. Arterial stiffness and hypertension are major risk factors for heart attack and stroke in the aged population. Arterial stiffness generally describes the elasticity or hardness of the arteries. The stiffness of arteries influences how hard the heart has to work to pump blood through the body. Hypertension generally refers to abnormally high blood pressure. Blood pressure is the force of blood pushing against the walls of arteries. Over time, arterial stiffness and hypertension can weaken and drainage the cardiovascular system leading to heart attack and stroke.

Current antihypertensive drugs are primarily designed to reduce peripheral resistance and are not adequate to alter the pathological process of arterial stiffening or arterial stiffness-related hypertension. Peripheral resistance is the resistance of the arteries to blood flow. As arteries constrict, peripheral resistance increases and as arteries dilate, peripheral resistance decreases. Peripheral resistance is determined by factors such as: (i) autonomic activity, whereas sympathetic activity constricts peripheral arteries; (ii) pharmacologic agents, such as vasoconstrictor drugs which increase peripheral resistance and vasorlition drugs which decrease peripheral resistance; and (iii) blood viscosity, whereas increased viscosity increases peripheral resistance.

DNA demethylation is a physiological process that maintains transcriptional activity of genes. An increase in methylation in the promoter region of a gene diminishes the promoter activity and gene transcription. DNA methylation is increased with age and the prevalence of arterial stiffness and hypertension are also increased with age. Physiologically, an appropriate methylation level is maintained by the balanced methylase and demethylase activity.

The Klotho gene was originally identified as a putative aging-suppressor gene in mice that extended life span when overexpressed and induced a premature aging syndrome when disrupted. Subsequently, the Klotho gene was found to be involved in numerous aging-associated pathologies, including chronic kidney disease, diabetes, cancers, and cardiovascular diseases. A deficiency of the Klotho gene can cause arterial stiffness. Further, Klotho protein (also referred to herein as “Klotho”) levels decrease with age while the prevalence of arterial stiffness and hypertension increase with age. For example, at age 70 years, the serum level of Klotho protein in a human is only about one half of what it was at age 40 years, Moreover, the serum Klotho protein level is significantly decreased in humans with arterial stiffness and chronic kidney diseases.

Arterial stiffening is an independent predictor of cardiovascular outcomes, such as hypertension, myocardial infarction, cognitive decline in aging, stroke, and kidney diseases. However, the relationship between DNA methylation and aging-related arterial stiffening and hypertension has not been previously understood. For example, it has not been known if increased methylation could lead to arterial stiffening and hypertension, or if increased demethylation could attenuate arterial stiffening and hypertension. The present work addresses these questions.

Klotho (or alpha-Klotho, a-Klotho, etc.) is a recently characterized protein encoded by the KL (or klotho) gene, located on human chromosome 13. Two transcripts that arise from a single klotho gene through alternative RNA splicing have been identified. See FIGS. 1 and 2. The first transcript is predicted to encode Klotho isoform 1—a full-length, 1,012 amino acid, single-pass transmembrane-membrane protein, with a short cytoplasmic tail (human residues 1003-1012), a transmembrane (TM) domain (human residues 982-1002), and extracellular region or domain (human residues 1-981) comprising two largely homologous (internal repeat) domains (termed KL1 (human residues 56-506, which is 450 residues long) and KL2 (human residues 515-953, which is 438 residues long), which each share 20%-40% amino acid sequence homology to β-glucosidases, but may lack similar levels of glucosidase catalytic activity), and a signal sequence (SS) domain (human residues 1-33). The SS, KL1, and KL2 domain-containing extracellular region (human residues 1-981) may be enzymatically cleaved by α/β-secretases, and released into the circulatory stream as a 130 kDa circulating protein, termed soluble klotho (or sKlotho, s-Klotho, alpha soluble-Klotho, etc.). The extracellular region can also be cleaved into separate 68 kDa protein (KL1+SS) and 64 kDa protein (KL2).

The second transcript, a splicing variant of alpha-klotho mRNA, encodes a second isoform of Klotho protein corresponding mainly to the KL1 domain. The internal splice donor site is thought to be located in exon 3 of the klotho gene. The resultant alternatively spliced transcript contains a 50 bp insertion after exon 3, with an in-frame translation stop codon at the end thereof. The expressed protein product is secreted into the circulation and is termed secreted Klotho (or Klotho isoform 2), which differs from the canonical sequence of isoform 1 at amino acid residues 535-549: DTTLSQFTDLNVYLW→SQLTKPISSLTKPYH, and with amino acid residues 550-1012 missing.

Accordingly, there may be a number of different Klotho proteins in the circulation at any given time, depending on gene expression, RNA splicing, and enzymatic cleavage. Despite the existence of various forms of alpha-Klotho protein, only the full length, membrane-bound, isoform 1 is known to form a complex with fibroblast growth factor (FGF) receptors and functions as an obligatory co-receptor for FGF23—a bone-derived hormone that induces phosphate excretion into urine and which has a regulatory role on Pᵢ and vitamin D metabolism.

Klotho is highly expressed in the kidney, brain, and to a lesser extent in other organs, and may also be found in the cerebrospinal fluid and urine of mammals. Circulating levels of soluble Klotho proteins in mammals are thought to decrease with age. In addition, Klotho-deficient mice exhibit accelerated aging phenotypes, whereas over-expression of klotho in mice has been shown to extend lifespan. In addition, Klotho has been implicated in a number of cellular processes related to aging. In light of the foregoing, a developing hypothesis states that soluble Klotho may function as an anti-aging compound in the human body.

The Klotho gene was originally identified as a putative aging-suppressor gene in mice that extended life span when overexpressed and induced a premature aging syndrome when disrupted. Subsequently, the Klotho gene was found to be involved in numerous aging-associated pathologies, including chronic kidney disease, diabetes, cancers, cognitive decline, sarcopenia and cardiovascular diseases. For example, a deficiency of the Klotho gene can cause arterial stiffness. Further, Klotho protein levels decrease with age while the prevalence of arterial stiffness and hypertension increase with age. For example, at age 70 years, the serum level of Klotho protein in a human is only about one half of what it was at age 40 years. Moreover, the serum Klotho protein level is significantly decreased in humans with arterial stiffness and chronic kidney diseases.

Aging is an inevitable and progressive biological process resulting in dysfunction and destruction of almost all tissues and organs, ultimately resulting in death. The aging of the human body, for instance, is associated with the decline of cellular function, which can lead to the development of a variety of diseases. Aging is thought to be driven by a tightly regulated and complex interplay between genetic, epigenetic, and acquired factors and is typically characterized by an increase in senescence, a quantitative and qualitative decrease in stem cells, and abnormal structure at tissue levels. For instance, an increase in DNA methylation in the promoter region of a gene diminishes the promoter activity and gene transcription. DNA demethylation is a physiological process that maintains transcriptional activity of genes, while DNA methylation is increased with age and the prevalence of arterial stiffness and hypertension are also increased with age. Physiologically, an appropriate methylation level is maintained by the balanced methylase and demethylase activity.

As the so-called “baby boomers” generation continues to advance in age, the population of aging individuals (e.g., age 60-65) is rapidly increasing globally. The increased demand for health care for this aging population places significant financial burden on any healthcare system. Molecular compounds (or so-called “small molecules”) provide promising therapeutic agents to counter age-related health conditions. Developing strategies and health intervention methods based on the production and purification of compounds that increase klotho gene expression, and the administration of such compounds to subjects may help to ameliorate this situation and the problems associated therewith. Developing strategies and health intervention methods based on the administration of small molecules that increase Klotho protein levels through increasing klotho gene expression to subjects, especially humans and/or within an increasing aging population, may help to ameliorate this situation.

Currently, there is not a commercial small molecule product or treatment method for increasing Klotho protein levels through increasing klotho gene expression, especially products and methods approved by the U.S. Food and Drug Administration (FDA). To date, all relevant data is connected to pre-clinical research studies in animal models. For example, King et al. published the Identification of novel small molecules that elevate Klotho expression (Biochem J. 2012 January 1; 441(1): 453-461), Jung et al. published the Induction of anti-aging gene klotho with a small chemical compound that demethylates CpG islands (Oncotarget, 2017, Vol. 8, (No. 29), pp: 46745-46755), Chen and Sun published the Activation of DNA demethylases attenuates aging-associated arterial stiffening an hypertension (Aging Cell. 2018; e12762), and US 2018/0338951 discloses Treatments for arterial stiffening, hypertension and anti-aging. However, each of the foregoing references, the entirety of each of which is incorporated by reference herein, falls short of providing a compound for FDA approved administration. For example, King et al. teaches a Compound H, which is deficient in therapeutic efficacy.

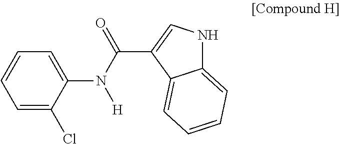 [SMILES: O=C(Nc1ccccc1Cl)c1c[nH]c2ccccc12]

Accordingly, there are a number of short-comings in the art that can be addressed by the development, production, manufacture, and administration of small molecules or compounds for increasing Klotho protein levels through increasing klotho gene expression.

### BRIEF SUMMARY

Embodiments of the present disclosure solve one or more of the foregoing or other problems in the art with compounds (e.g., small molecules) and compositions including the same for increasing klotho gene expression, particularly for increasing circulating and/or soluble Klotho protein levels through increasing klotho gene expression, and methods of manufacturing and using the same.

Illustratively, the novel compounds are, or function as, therapeutic agents that activate, enhance, or increase klotho gene expression, thereby increasing circulating and/or soluble Klotho protein levels in mammals. Accordingly, some embodiments include a method of increasing klotho gene expression, or a method for increasing circulating and/or soluble Klotho protein levels through increasing klotho gene expression, the methods comprising administering a composition of the present disclosure to a mammalian subject.

Illustrative embodiments of the present disclosure include a compound according to Formula I:

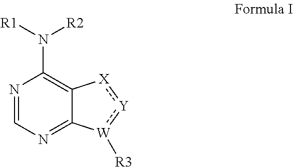

or a stereoisomer, tautomer or pharmaceutically acceptable salt thereof.

In various embodiments, X is (selected from (the group consisting of)) N, S, or C—R₄, wherein R₄ is (selected from (the group consisting of)) H or C1-C5 alkyl. In some embodiments, X is N. In some embodiments, X is S. In some embodiments, X is CH. In some embodiments, X is C1-C5 alkyl.

In various embodiments, Y is (selected from (the group consisting of)) N or C—R₄, wherein R₄ is (selected from (the group consisting of)) H or C1-C5 alkyl. In some embodiments, Y is N. In some embodiments, Y is CH. In some embodiments, Y is C1-C5 alkyl.

In various embodiments, the bond between X—Y is a single bond or a double bond. In some embodiments, the bond between X—Y is a single bond. In some embodiments, the bond between X-Y is a double bond.

In various embodiments, W is (selected from (the group consisting of)) N, S, or C. In some embodiments, W is N. In some embodiments, W is S. In some embodiments, W is C.

In various embodiments, the bond between Y—W is a single bond or a double bond. In some embodiments, the bond between Y—W is a single bond. In some embodiments, the bond between Y—W is a double bond.

In various embodiments, R₁ is (selected from (the group consisting of)) H, CH₃, or, together with R₂, forms substituted or unsubstituted heterocyclic amine. In some embodiments, R₁ is H. In some embodiments, R₁ is CH₃. In various embodiments, R₁, together with R₂, forms substituted or unsubstituted heterocyclic amine.

In various embodiments, R₂ is (selected from (the group consisting of)):

(CH₂)_{Z}—(CR₅R₆)_{V}—R₇, wherein Z is an integer from 0-2, V is an integer from 0-2, R₅ is (selected from (the group consisting of)) H or CH₃, R₆ is (selected from (the group consisting of)) H or CH₃; and R₇ is (selected from (the group consisting of)):

(i) saturated or unsaturated C3-C8 substituted or unsubstituted cycloalkyl or bicycloalkyl (e.g., bicyclo octane, preferably, bicyclo(2.2.2)octane), optionally substituted at one or more (ring) positions and each (ring) substituent is (selected from (the group consisting of)) halo, aryl (phenyl or benzyl), or branched or unbranched C1-C3 alkyl;

(ii) substituted or unsubstituted aryl (phenyl or benzyl), optionally substituted at one or more (1 or 2) (ring) positions and each (ring) substituent is (selected from (the group consisting of))halo or branched or unbranched C1-C3 alkyl; and/or

(iii) branched or unbranched C1-C3 alkyl (e.g., isopropyl);

C3-C7 substituted or unsubstituted cycloalkyl (e.g., 2-phenylcyclopropyl), optionally substituted at one or more (ring) positions and each (ring) substituent is (selected from (the group consisting of)) substituted or unsubstituted aryl (phenyl or benzyl), branched or unbranched C1-C3 substituted or unsubstituted alkyl; and/or

together with R₁, forms substituted or unsubstituted heterocyclic amine.

In some embodiments, R₂ is not H. In some embodiments, R₂ is not CH₃. In some embodiments, R₂ is not, one or more of, alkyl, ethyl, propyl, isopropyl, or butyl. In some embodiments, R₂ is not aryl, phenyl or benzyl. In some embodiments, R₂ is not carboxyl (COOH). In some embodiments, R₂ is not, one or more of, methanol, ethanol, propanol, or isopropanol.

In various embodiments, R₃ is (selected from (the group consisting of)): nothing, H, alkyl, cycloalkyl, aryl (phenyl or benzyl), nitrile, (CH₂)_{Z}CN, wherein Z is an integer from 1-3, branched or unbranched C1-C3 substituted or unsubstituted alkyl, C3-C7 substituted or unsubstituted cycloalkyl, aryl (phenyl or benzyl) optionally substituted at one or more (ring) positions and each (ring) substituent is (selected from (the group consisting of)) branched or unbranched C1-C3 substituted or unsubstituted alkyl, halo, or nitrile. In some embodiments, R₃ is nothing. In some embodiments, R₃ is H. In some embodiments, R₃ is alkyl. In some embodiments, R₃ is cycloalkyl. In some embodiments, R₃ is aryl (phenyl or benzyl). In some embodiments, R₃ is nitrile. In some embodiments, R₃ is (CH₂)_{Z}CN, wherein Z is an integer from 1-3. In some embodiments, R₃ is branched or unbranched C1-C3 substituted or unsubstituted alkyl. In some embodiments, R₃ is C3-C7 substituted or unsubstituted cycloalkyl. In some embodiments, R₃ is aryl (phenyl or benzyl) substituted at one or more (ring) positions and each (ring) substituent is (selected from (the group consisting of)) branched or unbranched C1-C3 substituted or unsubstituted alkyl, halo, or nitrile. In some embodiments, R₃ is aryl (phenyl or benzyl) substituted at one or more (ring) positions with branched or unbranched C1-C3 substituted or unsubstituted alkyl, halo, or nitrile.

In some embodiments, R₁ is H and R₂ is (CH₂)_{Z}—(CR₅R₆)_{V}—R₇, wherein Z is an integer from 1-2, V is 0, and R₇ is (selected from (the group consisting of):

saturated or unsaturated C4-C7 cycloalkyl, optionally substitute at one or more (ring) positions with one or more methyl, preferably saturated C4-C6 cycloalkyl, optionally substitute at one or more (ring) positions with one or more methyl, more preferably unsubstituted saturated C4-C6 cycloalkyl or saturated C6 cycloalkyl optionally substituted at one or more (ring) position with one or more methyl or fluoro, still more preferably monounsaturated C5-C7 cycloalkyl, preferably 2-(1-cycloalkenyl or 4-(1-cycloalkenyl, more preferably 2-(1-cyclopentenyl), 2-(1-cyclohexenyl), 2-(1-cycloheptenyl), 4-(1-cyclopentenyl), 4-(1-cyclohexenyl), or 4-(1-cycloheptenyl);

bicycloalkyl, preferably bicyclo octane, more preferably, bicyclo(2.2.2)octane;

aryl (or phenyl or benzyl), preferably substituted at one or more (ring) positions with halo, preferably chloro, more preferably 3-chlorobenzyl or 2,3-dichlorobenzyl;

isopropyl; and/or

C3 cycloalkyl, preferably substituted with aryl (or phenyl or benzyl), preferably 2-phenylcyclopropyl.

In some embodiments, R₁ is H and R₂ is (CH₂)_{Z}—(CR₅R₆)_{V}—R₇, wherein Z is 1, V is 1, R₅ is CH₃, R₆ is CH₃, and R₇ is monounsaturated cyclohexyl, preferably 2-(1-cyclohexenyl).

In some embodiments, R₁ is CH₃ and R₂ is (CH₂)_{Z}—(CR₅R₆)_{V}—R₇, Z is 2, V is 0, and R₇ is cyclohexyl.

In some embodiments, R₁ together with R₂, forms substituted or unsubstituted heterocyclic amine, preferably, substituted azepane, more preferably 4,4-diethylazepane. In some embodiments, R₁ and R₂ form a substituted or unsubstituted, saturated or unsaturated heterocyclic amine, preferably a substituted azepane, aziridine, azetidine, pyrrolidine, piperidine, or azocane, more preferably azepane, still more preferably 4,4-diethyl azepane.

In some embodiments:

X is N, Y is C—R₄, R₄ is H, W is N, the bond between X—Y is a double bond, and the bond between Y—W is a single bond, and wherein R₃ is preferably alkyl or cycloalkyl, more preferably ethyl or cyclopropyl;

X is C—R₄, Y is C—R₄, R₄ is H, W is N, the bond between X—Y is a double bond, and the bond between Y—W is a single bond, and preferably, wherein R₃ is H;

X is C—R₄, R₄ is H, Y is N, W is N, the bond between X—Y is a double bond, and the bond between Y—W is a single bond, and preferably, wherein R₃ is preferably (i) alkyl, more preferably methyl, (ii) substituted or unsubstituted aryl, phenyl or benzyl, still more preferably fluorophenyl or fluorobenzyl, or (iii) nitrile, preferably ethanenitrile or ethyl cyanide;

X is N or C—R₄, Y is C—R₄, each R₄ is, independently, H or CH₃, W is S, the bond between X—Y is a double bond, and the bond between Y—W is a single bond, and wherein R₃ is nothing;

X is C—R₄, Y is C—R₄, each R₄ is, independently, H or CH₃, W is S, the bond between X—Y is a double bond, and the bond between Y—W is a single bond, and wherein R₃ is nothing;

X is N, Y is C—R₄, R₄ is H, W is S, the bond between X—Y is a double bond, and the bond between Y—W is a single bond, and wherein R₃ is nothing;

X is N, Y is C—R₄, R₄ is H, W is N, the bond between X—Y is a double bond, and the bond between Y—W is a single bond, and wherein R₃ is preferably C2-C3 alkyl or cycloalkyl, preferably, ethyl or cyclopropyl; or

X is S, Y is C—R₄, R₄ is H, W is C, the bond between X—Y is a single bond, and the bond between Y—W is a double bond, and wherein R₃ is preferably H.

In some embodiments, X is N, Y is C—R₄, R₄ is H, W is N, the bond between X—Y is a double bond, and the bond between Y—W is a single bond, and wherein R₃ is preferably alkyl or cycloalkyl, more preferably ethyl or cyclopropyl.

In some embodiments, X is C—R₄, Y is C—R₄, R₄ is H, W is N, the bond between X—Y is a double bond, and the bond between Y—W is a single bond, and preferably, wherein R₃ is H.

In some embodiments, X is C—R₄, R₄ is H, Y is N, W is N, the bond between X—Y is a double bond, and the bond between Y—W is a single bond, and preferably, wherein R₃ is preferably (selected from (the group consisting of)) (i) alkyl, more preferably methyl, (ii) substituted or unsubstituted aryl, phenyl or benzyl, still more preferably fluorophenyl or fluorobenzyl, or (iii) nitrile, preferably ethanenitrile or ethyl cyanide.

In some embodiments, X is N or C—R₄, Y is C—R₄, each R₄ is, independently, H or CH₃, W is S, the bond between X—Y is a double bond, and the bond between Y—W is a single bond, and wherein R₃ is nothing.

In some embodiments, X is C—R₄, Y is C—R₄, each R₄ is, independently, H or CH₃, W is S, the bond between X—Y is a double bond, and the bond between Y—W is a single bond, and wherein R₃ is nothing.

In some embodiments, X is N, Y is C—R₄, R₄ is H, W is S, the bond between X—Y is a double bond, and the bond between Y—W is a single bond, and wherein R₃ is nothing.

In some embodiments, X is N, Y is C—R₄, R₄ is H, W is N, the bond between X—Y is a double bond, and the bond between Y—W is a single bond, and wherein R₃ is preferably (selected from (the group consisting of)) C2-C3 alkyl or cycloalkyl, preferably, ethyl or cyclopropyl.

In some embodiments, X is S, Y is C—R₄, R₄ is H, W is C, the bond between X—Y is a single bond, and the bond between Y—W is a double bond, and wherein R₃ is preferably H.

In some embodiments, X is N, Y is C—R₄, R₄ is H, W is N, the bond between X—Y is a double bond, the bond between Y—W is a single bond, R₁ is H, R₂ is (CH₂)_{Z}—(CR₅R₆)_{V}—R₇, wherein Z is 1, V is 0, and R₇ is (selected from (the group consisting of)) unsubstituted monounsaturated C6 cycloalkyl or unsubstituted cyclohexenyl, preferably 4-(1-cyclohexenyl), more preferably R-4-(1-cyclohexenyl) or S-4-(1-cyclohexenyl), and R₃ is cyclopropyl.

In some embodiments, X is N, Y is C—R₄, R₄ is H, W is N, the bond between X—Y is a double bond, and the bond between Y—W is a single bond, R₃ is (selected from (the group consisting of)) alkyl or cycloalkyl, preferably ethyl or cyclopropyl, R₁ is H and R₂ is (CH₂)_{Z}—(CR₅R₆)ᵥ-R₇, wherein Z is an integer from 1-2, V is 0, and R₇ is (selected from (the group consisting of)):

unsubstituted saturated C4-C6 cycloalkyl;

saturated C6 cycloalkyl substituted at one (ring) positions with fluoro, methyl, or dimethyl, preferably 1-fluorocyclohexyl, 1-methylcyclohexyl, or 4,4-dimethylcyclohexyl;

unsubstituted monounsaturated C5-C7 cycloalkyl, preferably 2-(1-cycloalkenyl or 4-(1-cycloalkenyl, more preferably 2-(1-cyclopentenyl), 2-(1-cyclohexenyl), 2-(1-cycloheptenyl), 4-(1-cyclopentenyl), 4-(1-cyclohexenyl), or 4-(1-cycloheptenyl);

bicyclo octane, more preferably, bicyclo(2.2.2)octane;

aryl (or phenyl or benzyl) substituted at one or two (ring) positions with halo, preferably chloro, more preferably 3-chlorobenzyl or 2,3-dichlorobenzyl;

isopropyl; and/or

C3 cycloalkyl substituted with aryl (or phenyl or benzyl), preferably 2-phenylcyclopropyl.

Illustrative embodiments of the present disclosure include a compound according to Formula Ia:

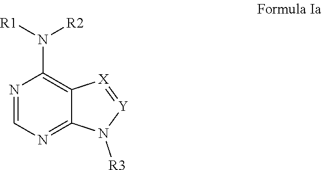

or a stereoisomer, tautomer or pharmaceutically acceptable salt thereof, wherein X, Y, R₁, R₂, and R₃ are as described for Formula I.

Illustrative embodiments of the present disclosure include a compound according to Formula II:

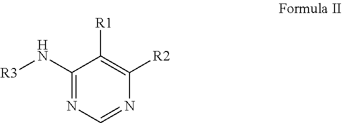

or a stereoisomer, tautomer or pharmaceutically acceptable salt thereof.

In various embodiments, R₁ is (selected from (the group consisting of) H or NH₂. In some embodiments, R₁ is H. In some embodiments, R₁ is NH₂.

In various embodiments, R₂ is (selected from (the group consisting of) saturated or unsaturated, heterocyclic amine or heterocyclic diamine. In some embodiments, R₂ is saturated or unsaturated heterocyclic amine. In some embodiments, R₂ is saturated or unsaturated heterocyclic diamine.

In some embodiments, R₂ is saturated heterocyclic amine, preferably N-pyrrolidine or N-piperidine, or unsaturated heterocyclic diamine, preferably N-pyrazole.

In some embodiments, when R₁ is H, then R₂ is N-pyrrolidine or N-piperidine.

In some embodiments, when R₁ is NH₂, then R₂ is N-pyrazole.

In various embodiments, R₃ is (CH₂)₂-R₇, wherein R₇ is unsaturated cycloalkyl.

In some embodiments, R₇ is monounsaturated C6-C7 cycloalkyl, preferably cyclohexenyl, more preferably 2-(1-cyclohexenyl), or cycloheptenyl, more preferably 2-(1-cycloheptenyl).

In some embodiments, the compound of Formula II is selected from the group consisting of Formulas IIa-IId:

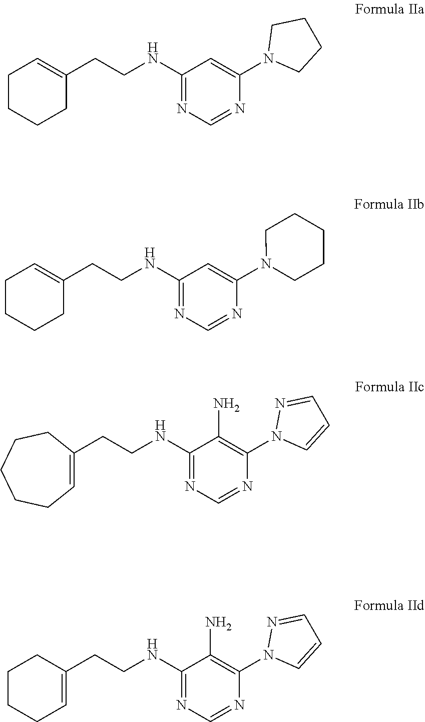 [SMILES: C1=C(CCNc2cc(N3CCCC3)ncn2)CCCC1 ; C1=C(CCNc2cc(N3CCCCC3)ncn2)CCCC1 ; Nc1c(NCCC2=CCCCCC2)ncnc1-n1cccn1 ; Nc1c(NCCC2=CCCCC2)ncnc1-n1cccn1]

or a stereoisomer, tautomer or pharmaceutically acceptable salt thereof.

Illustrative embodiments of the present disclosure include a compound according to Formula III:

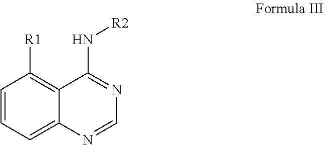

or a stereoisomer, tautomer or pharmaceutically acceptable salt thereof, wherein R₁ is halo; and R₂ is (CH₂)₂-R₇ wherein R₇ is unsaturated cycloalkyl. In some embodiments, R₁ is fluoro and R₇ is unsaturated cycloalkyl, preferably monounsaturated cycloalkyl, more preferably cyclohexenyl, more preferably 2-(1-cyclohexenyl), still most preferably, wherein the compound is according to Formula IIIa:

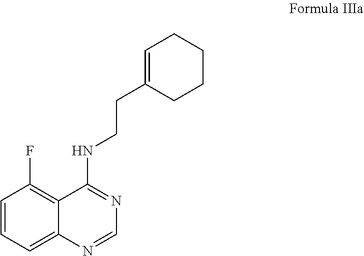 [SMILES: Fc1cccc2ncnc(NCCC3=CCCCC3)c12]

In some embodiments, the compound according to Formula I can be one of Compounds 1-49 of Table 1.

Table 1. Nonlimiting examples of illustrative compounds:

TABLE 1

| Compound | | | Molecular |
|---|---|---|---|
| Number | Structure | Mol. Wt. | Formula |
| 1 | 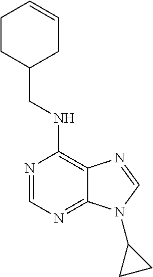 [SMILES: C1=CCC(CNc2ncnc3c2ncn3C2CC2)CC1] | 269.4 | C15H19N5 |
| 2 | 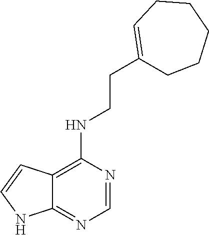 [SMILES: C1=C(CCNc2ncnc3[nH]ccc23)CCCCC1] | 256.4 | C15H20N4 |
| 3 | 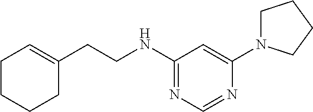 [SMILES: C1=C(CCNc2cc(N3CCCC3)ncn2)CCCC1] | 272.4 | C16H24N4 |
| 4 | 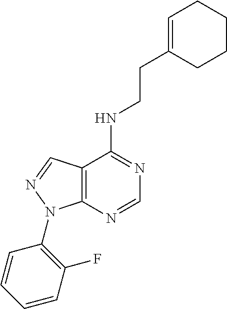 [SMILES: Fc1ccccc1-n1ncc2c(NCCC3=CCCCC3)ncnc21] | 337.4 | C19H20N5F |
| 5 | 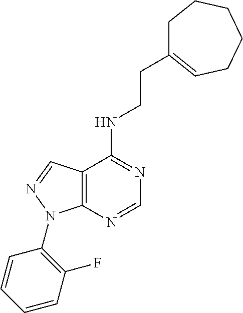 [SMILES: Fc1ccccc1-n1ncc2c(NCCC3=CCCCCC3)ncnc21] | 351.4 | C20H22N5F |
| 6 | 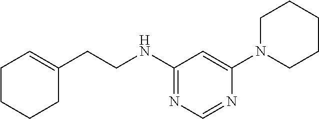 [SMILES: C1=C(CCNc2cc(N3CCCCC3)ncn2)CCCC1] | 286.4 | C17H26N4 |
| 7 | 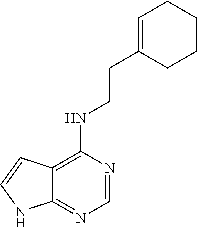 [SMILES: C1=C(CCNc2ncnc3[nH]ccc23)CCCC1] | 242.3 | C14H18N4 |
| 8 | 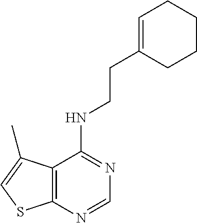 [SMILES: Cc1csc2ncnc(NCCC3=CCCCC3)c12] | 273.4 | C15H19N3S |
| 9 | 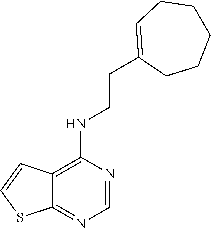 [SMILES: C1=C(CCNc2ncnc3sccc23)CCCCC1] | 273.4 | C15H19N3S |
| 10 | 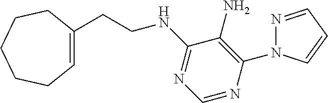 [SMILES: Nc1c(NCCC2=CCCCCC2)ncnc1-n1cccn1] | 298.4 | C16H22N6 |
| 11 | 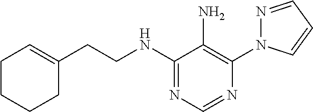 [SMILES: Nc1c(NCCC2=CCCCC2)ncnc1-n1cccn1] | 284.4 | C15H20N6 |
| 12 | 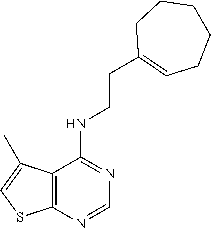 [SMILES: Cc1csc2ncnc(NCCC3=CCCCCC3)c12] | 287.4 | C16H21N3S |
| 13 | 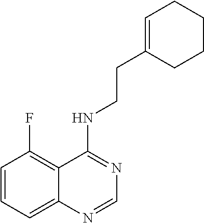 [SMILES: Fc1cccc2ncnc(NCCC3=CCCCC3)c12] | 271.3 | C16H18N3F |
| 14 | 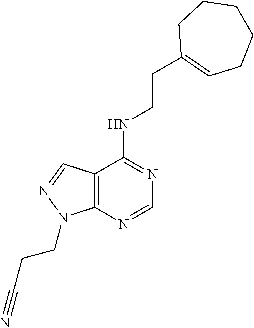 [SMILES: N#CCCn1ncc2c(NCCC3=CCCCCC3)ncnc21] | 310.4 | C17H22N6 |
| 15 | 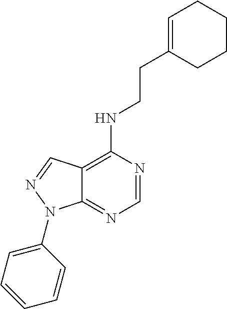 [SMILES: C1=C(CCNc2ncnc3c2cnn3-c2ccccc2)CCCC1] | 319.4 | C19H21N5 |
| 16 | 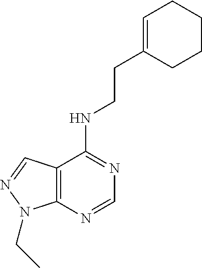 [SMILES: CCn1ncc2c(NCCC3=CCCCC3)ncnc21] | 271.4 | C15H21N5 |
| 17 | 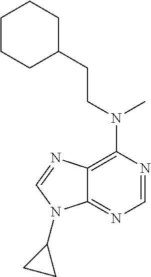 [SMILES: CN(CCC1CCCCC1)c1ncnc2c1ncn2C1CC1] | 299.4 | C17H25N5 |
| 18 | 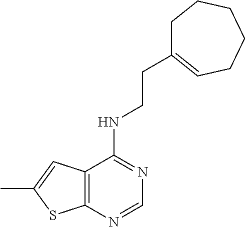 [SMILES: Cc1cc2c(NCCC3=CCCCCC3)ncnc2s1] | 287.4 | C16H21N3S |
| 19 | 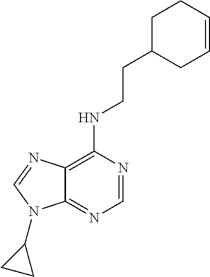 [SMILES: C1=CCC(CCNc2ncnc3c2ncn3C2CC2)CC1] | 283.4 | C16H21N5 |
| 20 | 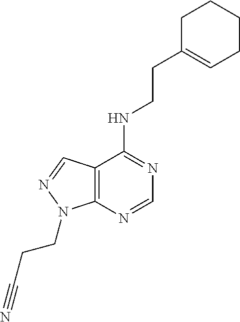 [SMILES: N#CCCn1ncc2c(NCCC3=CCCCC3)ncnc21] | 296.4 | C16H20N6 |
| 21 | 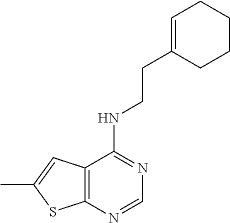 [SMILES: Cc1cc2c(NCCC3=CCCCC3)ncnc2s1] | 273.4 | C15H19N3S |
| 22 | 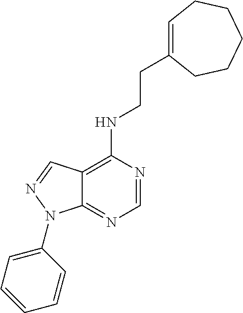 [SMILES: C1=C(CCNc2ncnc3c2cnn3-c2ccccc2)CCCCC1] | 333.4 | C20H23N5 |
| 23 | 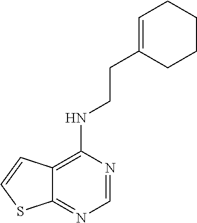 [SMILES: C1=C(CCNc2ncnc3sccc23)CCCC1] | 259.4 | C14H17N3S |
| 24 | 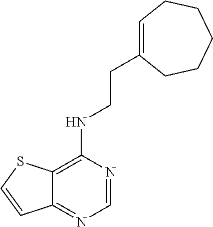 [SMILES: C1=C(CCNc2ncnc3ccsc23)CCCCC1] | 273.4 | C15H19N3S |
| 25 | 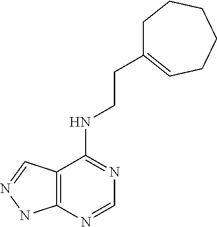 [SMILES: C1=C(CCNc2ncnc3[nH]ncc23)CCCCC1] | 271.4 | C15H21N5 |
| 26 | 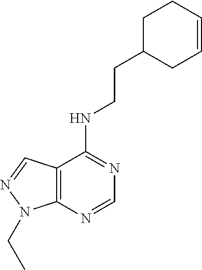 [SMILES: CCn1ncc2c(NCCC3CC=CCC3)ncnc21] | 271.4 | C15H21N5 |
| 27 | 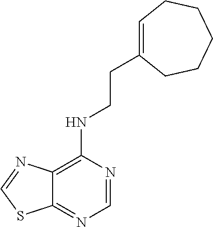 [SMILES: C1=C(CCNc2ncnc3scnc23)CCCCC1] | 274.4 | C14H18N4S |
| 28 | 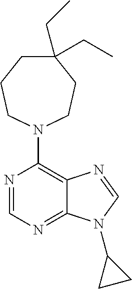 [SMILES: CCC1(CC)CCCN(c2ncnc3c2ncn3C2CC2)CC1] | 313.4 | C18H27N5 |
| 29 | 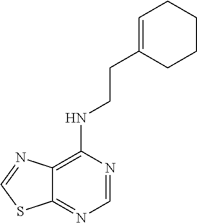 [SMILES: C1=C(CCNc2ncnc3scnc23)CCCC1] | 260.4 | C13H16N4S |
| 30 | 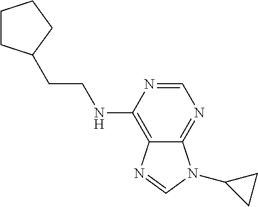 [SMILES: c1nc(NCCC2CCCC2)c2ncn(C3CC3)c2n1] | 271.4 | C15H21N5 |
| 31 | 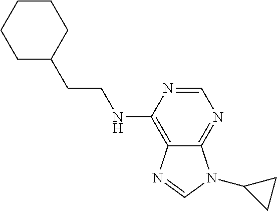 [SMILES: c1nc(NCCC2CCCCC2)c2ncn(C3CC3)c2n1] | 285.4 | C16H23N5 |
| 32 | 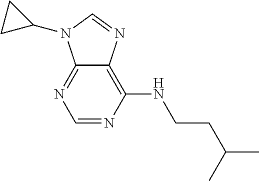 [SMILES: CC(C)CCNc1ncnc2c1ncn2C1CC1] | 245.3 | C13H19N5 |
| 33 | 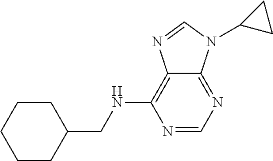 [SMILES: c1nc(NCC2CCCCC2)c2ncn(C3CC3)c2n1] | 271.4 | C15H21N5 |
| 34 | 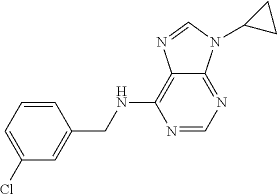 [SMILES: Clc1cccc(CNc2ncnc3c2ncn3C2CC2)c1] | 299.8 | C15H14N5Cl |
| 35 | 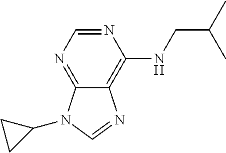 [SMILES: CC(C)CNc1ncnc2c1ncn2C1CC1] | 231.3 | C12H17N5 |
| 36 | 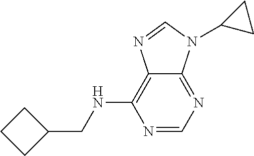 [SMILES: c1nc(NCC2CCC2)c2ncn(C3CC3)c2n1] | 243.3 | C13H17N5 |
| 37 | 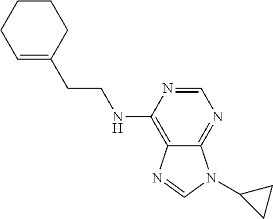 [SMILES: C1=C(CCNc2ncnc3c2ncn3C2CC2)CCCC1] | 283.4 | C16H21N5 |
| 38 | 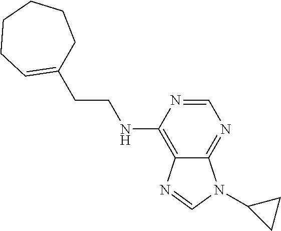 [SMILES: C1=C(CCNc2ncnc3c2ncn3C2CC2)CCCCC1] | 297.4 | C17H23N5 |
| 39 | 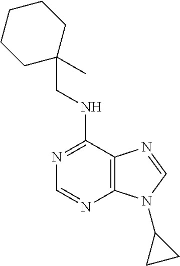 [SMILES: CC1(CNc2ncnc3c2ncn3C2CC2)CCCCC1] | 285.4 | C16H23N5 |
| 40 | 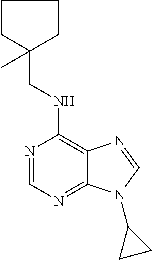 [SMILES: CC1(CNc2ncnc3c2ncn3C2CC2)CCCC1] | 271.4 | C15H21N5 |
| 41 | 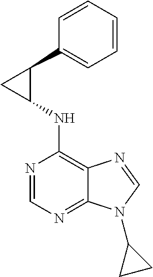 [SMILES: c1ccc([C@@H]2C[C@H]2Nc2ncnc3c2ncn3C2CC2)cc1] | 291.4 | C17H17N5 |
| 42 | 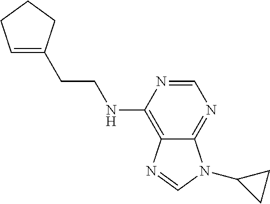 [SMILES: C1=C(CCNc2ncnc3c2ncn3C2CC2)CCC1] | 269.4 | C15H19N5 |
| 43 | 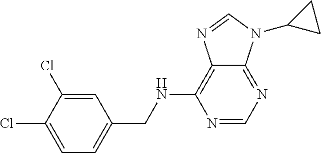 [SMILES: Clc1ccc(CNc2ncnc3c2ncn3C2CC2)cc1Cl] | 334.2 | C15H13N5Cl2 |
| 44 | 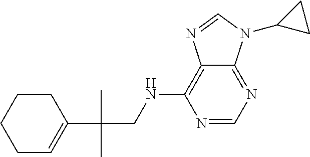 [SMILES: CC(C)(CNc1ncnc2c1ncn2C1CC1)C1=CCCCC1] | 311.4 | C18H25N5 |
| 45 | 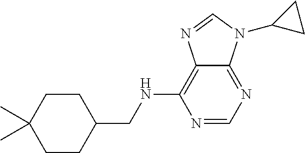 [SMILES: CC1(C)CCC(CNc2ncnc3c2ncn3C2CC2)CC1] | 299.4 | C17H25N5 |
| 46 | 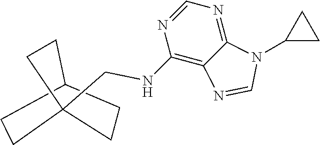 [SMILES: c1nc(NCC23CCC(CC2)CC3)c2ncn(C3CC3)c2n1] | 297.4 | C17H23N5 |
| 47 | 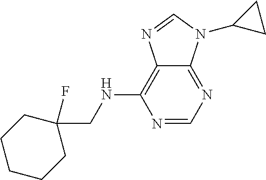 [SMILES: FC1(CNc2ncnc3c2ncn3C2CC2)CCCCC1] | 289.4 | C15H20N5F |
| 48 | 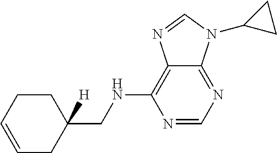 [SMILES: C1=CC[C@@H](CNc2ncnc3c2ncn3C2CC2)CC1] | 269.4 | C15H19N5 |
| 49 | 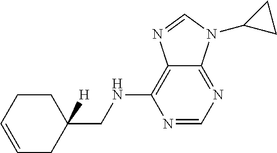 [SMILES: C1=CC[C@@H](CNc2ncnc3c2ncn3C2CC2)CC1] | 269.4 | C15H19N5 |

Furthermore, all compounds of the present disclosure which exist in free base or acid form can be converted to their pharmaceutically acceptable salts by treatment with the appropriate inorganic or organic base or acid by methods known to one skilled in the art. Salts of the compounds of the present disclosure can be converted to their free base or acid form by standard techniques.

Various “R-groups” are disclosed in the foregoing chart and in FIG. 1. An R-group according to the present disclosure may be any of the R-groups disclosed in the foregoing chart or in FIG. 1. Those skilled in the art will appreciate that other R-groups may be suitable as substituents for the disclosed R-group. Illustratively, an R-group may be selected from the group consisting of: nothing, hydrogen, halogen, ketone, sulfur, sulfone, sulfoxide, OH, CH₂OH, CONH₂, CN, NO₂, CHO, CH₃CH(OH), OCH₂COOH, or an (optionally substituted) alkyl, cycloalkyl, alkoxy, thioalkyl, sulfoxyalkyl, sulfonylalkyl, alkylene dioxy, haloalkyl, haloalkoxy, acetoxy, N(alkyl)₂, benzyl, benzyloxy, α,α-dimethylbenzyl, acetyl, or aromatic ring system—e.g., said optionally substituted aromatic ring system being optionally selected from the group consisting of phenyl, phenoxy, and heterocyclic aryl, wherein up to 5 substituents are optionally present on the aromatic ring system, and each substituent is optionally independently selected from the group consisting of: hydrogen, alkyl, cycloalkyl, halogen, alkoxy, thioalkyl, sulfoxyalkyl, sulfonylalkyl, alkylene dioxy, haloalkyl, haloalkoxy, OH, CH₂OH, CONH₂, CN, acetoxy, N(alkyl)₂, NO₂, CHO, CH₃CH(OH), acetyl, OCH₂COOH, (CH₂)₂SO₃H, or CHZCOOH, wherein Z is one of the group consisting of H, CH₃, CH(CH₃)₂, CH₂C₆H₅, CH₂CH(CH₃)₂, and CH(CH₃)CH₂CH₃; or R_{A} and R_{B} together are a cycloalkyl.

Some embodiments are directed to a (pharmaceutical) composition comprising a pharmaceutically acceptable carrier or excipient and a compound of Formula I, II, or III.

Some embodiments are directed to a (pharmaceutical) medicament comprising a pharmaceutically acceptable carrier or excipient and a compound of Formula I, II, or III, or composition comprising the same.

Some embodiments are directed to a composition or medicament including a compound of Formula I, II, or III for use in (i) increasing klotho gene expression, (ii) increasing circulating and/or soluble Klotho protein levels, preferably through increasing klotho gene expression, (iii) treating Klotho protein deficiency, and/or (iv) treating other related conditions or affecting other related molecular mechanisms, in a mammal or mammalian subject (in need thereof). In some embodiments, the mammal or mammalian subject is human.

Some embodiments are directed to use of the compound of Formula I, II, or III, a pharmaceutical compositions comprising the same, or a medicament comprising the same, for (i) increasing klotho gene expression, (ii) increasing circulating and/or soluble Klotho protein levels, preferably through increasing klotho gene expression, (iii) treating Klotho protein deficiency, and/or (iv) treating other related conditions or affecting other related molecular mechanisms, in a mammal or mammalian subject (in need thereof). In some embodiments, the mammal or mammalian subject is human.

Some embodiments are directed to a method of (i) increasing klotho gene expression, (ii) increasing circulating and/or soluble Klotho protein levels, preferably through increasing klotho gene expression, (iii) treating Klotho protein deficiency, and/or (iv) treating other related conditions or affecting other related molecular mechanisms, in a mammal or mammalian subject (in need thereof), the method comprising administering a compound according to Formula I, II, or III, a pharmaceutical compositions comprising the same, or a medicament comprising the same, to the mammal or mammalian subject (in need thereof). In some embodiments, the mammal or mammalian subject is human.

Embodiments of the present disclosure are designed to be effective for increasing klotho gene expression in (mammalian) patients to which the novel compound(s), or composition(s) comprising the same, is/are administered. An increase in klotho gene expression can lead directly to an increase in circulating and/or soluble Klotho protein level(s). Those skilled in the art will appreciate that any disease or condition (particularly in older patients) that is caused, worsened, or exacerbated, in whole or in part, by (or associated with) low or diminished Klotho protein levels, may be addressed and/or treated (post-diagnosis or prophylactically) by administration of the novel compound(s), or composition(s) comprising the same, disclosed herein.

Some embodiments may include any of the features, options, and/or possibilities set out elsewhere in the present disclosure, including in other aspects or embodiments of the present disclosure. It is also noted that each of the foregoing, following, and/or other features described herein represent a distinct embodiment of the present disclosure. Moreover, combinations of any two or more of such features represent distinct embodiments of the present disclosure. Such features or embodiments can also be combined in any suitable combination and/or order without departing from the scope of this disclosure. Thus, each of the features described herein can be combinable with any one or more other features described herein in any suitable combination and/or order. Accordingly, the present disclosure is not limited to the specific combinations of exemplary embodiments described in detail herein.

Additional features and advantages of exemplary embodiments of the present disclosure will be set forth in the description that follows, and in part will be obvious from the description, or may be learned by the practice of such exemplary embodiments. The features and advantages of such embodiments may be realized and obtained by means of the instruments and combinations particularly pointed out in the appended claims. These and other features will become more fully apparent from the following description and appended claims, or may be learned by the practice of such exemplary embodiments as set forth hereinafter.

### BRIEF DESCRIPTION OF THE DRAWINGS

Several embodiments of the. present disclosure are hereby illustrated in the appended drawings. It is to be noted however, that the appended drawings only illustrate certain embodiments and are therefore not intended to be considered limiting of the scope of the present disclosure. In order to describe the manner in which the above-recited and other advantages and features of the present disclosure can be obtained, a more particular description of the embodiments briefly described above will be rendered by reference to specific embodiments thereof which are illustrated in the appended drawings. For better understanding, the like elements have been designated by like reference numbers throughout the figure(s). Understanding that these drawings depict only typical embodiments of the disclosure and are not therefore to be considered to be limiting of its scope, the disclosure will be described and explained with additional specificity and detail through the use of the accompanying drawing(s) in which:

FIG. 1 depicts 49 illustrative compounds and information associated with the same.

FIG. 2 illustrates change in systolic blood pressure over time in rats treated with illustrative Compound 49.

FIG. 3 illustrates change in diastolic blood pressure over time in rats treated with illustrative Compound 49.

FIG. 4A illustrates systolic blood pressure in male mice treated with illustrative Compound 49.

FIG. 4B illustrates diastolic blood pressure in male mice treated with illustrative Compound 49.

FIG. 4C illustrates mean blood pressure in male mice treated with illustrative Compound 49.

FIG. 4D illustrates grip strength in male mice treated with illustrative Compound 49.

FIG. 5A illustrates systolic blood pressure in female mice treated with illustrative Compound 49.

FIG. 5B illustrates diastolic blood pressure in female mice treated with illustrative Compound 49.

FIG. 5C illustrates mean blood pressure in female mice treated with illustrative Compound 49.

FIG. 5D illustrates grip strength in female mice treated with illustrative Compound 49.

FIG. 6A illustrates grip strength in male mice treated with illustrative Compound 49.

FIG. 6B illustrates grip strength in female mice treated with illustrative Compound 49.

FIG. 7A-7E graphically depicts the effects of the active agent (Compound H, a.k.a., Com H) on arterial pulse, wave velocity (PWV) and blood pressure (BP) in old mice in comparison to levels in untreated adult mice. (A) PWV as measured by 10-MHz Doppler probes. (B-E) pulse, systolic, diastolic, and mean BP, respectively, measured after 2 weeks of active agent treatment by the volume-pressure recording (“VPR”) tail-cuff method using a CODA 6 BP Monitoring System. Data are expressed as mean±standard error (SE) and analyzed by one-way ANOVA, n=6-7, *p<0.05. **p<0.01 vs adult mice; #p<0.05, ##p-<0.0.1 vs old mice. Bars indicate the standard error of the mean (SEM).

FIG. 8A-8C graphically depicts the effects of the active agent on DNA hypennethylation of Klotho gene in the kidney of old mice in comparison to levels in untreated adult mice. (A) DNA demethylase activity, tF6, with and without the active agent. (B) DNA methyltransferase activity, n=6, with and without the active agent. (C) DNA methylation index of Klotho gene, n=A, with and without the active agent Data are expressed as mean±SE and analyzed by one-way ANOVA, *p<0.05, **p<0.0.1 vs adult mice: #p<0.05, ##p<0.01 vs old mice. Bars indicate the SEM.

FIG. 9A-9C graphically depicts the effects of the active agent on full-length and secreted Klotho protein (“Klotho”) levels in the kidney and serum of old mice in comparison to levels in untreated adult mice. (A) western blot analysis of Klotho in kidney, n=4. (B) western blot analysis of Klotho in Serum, n=4. (C) Klotho mRNA expression in kidney, n=4. Data are expressed as mean±SE and analyzed by one-way ANOVA, *p<0.05, **p<0.01 vs adult mice; #p<0.05, ##p<0.01 vs old mice. Bars indicate the SEM.

FIG. 10A graphically depicts the effects of the active agent on the accumulation of collagen and degeneration of elastin in the aorta of old mice in comparison to levels in untreated adult mice. The histological and immunohistochemical staining results of collagen-1 and elastin are indicated by arrows, n=5. The active agent decreases the amount of collagen and increases the amount of elastin in old mice. Data are expressed as mean±SE and analyzed by one-way ANOVA, *p<0.05, **p<0.01 vs adult mice; #p<0.05, ##p<0.0 vs old mice. Bars indicate the SEM.

FIG. 10B graphically depicts western blot analysis of the effects of the active agent on collagen-1 and elastin in old mice, in comparison to levels in untreated adult mice, n=4. The western blot analysis confirms that the active agent decreases the amount of collagen and increases the amount of elastin in old mice. Data are expressed as mean±SE and analyzed by one-way ANOVA, *p<0.05, **p<0.01 vs adult mice; #p<0.05, ##p<0.01 vs old mice. Bars indicate the SEM. Tubulin is used as a control protein.

FIG. 11A graphically depicts by zymogram PAGE that the active agent significantly reduced the increases in arterial matrix metalloproteinase-2 (MMP-2) and matrix metalloproteinase-9 (MMP-9) activity and expression in old mice in comparison to levels in untreated adult mice. Data are expressed as mean±SE and analyzed by one-way ANOVA, n=4, *p<0.05, **p<0.01 vs adult mice; #p<0.05, ##p<0.01. vs old mice. Bars indicate the SEM.

FIG. 11B graphically depicts by western blot analysis that the active agent significantly reduced MMP-2 and MMP-9 expression in old mice in comparison to levels in untreated adult mice. Data are expressed as mean±SE and analyzed by one-way ANOVA, n=4, *p<0.05, **p<0.01 vs adult mice; #p<0.05, ##p<0.01 vs old mice. Bars indicate the SEM.

FIG. 12A-12E graphically depicts by western blot analysis the effects of the active agent on levels of arterial transforming growth factor-β1 (TGF-β1), transforming growth factor-β3 (TGF-β3), runt related transcription factor2 (RUNX2) and alkaline phosphalase (ALP) expression in old mice in comparison to untreated adult mice. (A) western blot analysis of TGF-β1, TGF-β3, RUNX2 and ALP. (B) quantification of TGF-β1 protein levels. (C) quantification of TGF-⊖3 protein levels. (D) quantification of RUNX2 protein levels. (E) quantification of ALP1 protein levels. Data are expressed as mean±SE and analyzed by one-way ANOVA, n=4, *p<0.05, **p<0.01 vs adult mice; #p-<0.05, ##p<0.01 vs old mice. Bars indicate the SEM.

FIG. 13A graphically depicts by western blot analysis the effects of the active agent on the Silent information regulator T1 enzyme (SirT1) activity and on its substrate acetyl p53 tumor suppressor protein (Ace-p53). The active agent increased SirT1 activity in the aorta of old mice. Data are expressed as mean±SE and analyzed by one-way ANOVA, n=4, *p<0.05, **p<0.01 vs adult mice; #p<0.05, ##p<0.01 vs old mice. Bars indicate the SEM.

FIG. 13B graphically depicts by western blot analysis the effects of the active agent on AMP-activated protein kinase (AMPK) and phospho-AMP-activated protein kinase (p-AMPK). Expression of both p-AMPK and AMPK was increased by the active agent in old mice. Data are expressed as mean±SE and analyzed by one-way ANOVA, n=4, *p<0.05, *p<0.05, **p<0.01 vs. adult mice; #p<0.05, ##p<0.01 vs old mice. Bars indicate the SEM.

FIG. 13C graphically depicts by western blot analysis the effects of the active agent on endothelial nitric oxide synthase (eNOS) and phosphor-endothelial nitric oxide synthase (p-eNOS). Expression of both p˜eNOS and eNOS was increased by the active agent in old mice. Data are expressed as mean±SE and analyzed by one-way ANOVA, n=4, *p<0.05, **p<0.01 vs adult mice; #p<0.05, ##p<0.01 vs old mice. Bars indicate the SEM.

FIG. 14 graphically depicts by western blot analysis that the active agent did not affect MMP2, MMP9, TGFIβ1, and TGFβ3 expression in mouse vascular aortic smooth muscle cells (MOVAS). MOVAS were treated with active agent, Klotho free (KL (−)) medium, and/or secreted Klotho (SKL) for 16 h and then harvested for western blot analysis. Data is expressed as mean±SE and analyzed by one-way ANOVA, n=4, *p<0.05, **p<0.01 vs regular medium; #p<0.05, ##p<0.01 vs KL(−) medium. Bars indicate the SEM.

FIG. 15A-15C graphically depicts by western blot that the active agent did not affect microtubule-associated protein light chain 3 (LC3) expression in aortas of old mice (A), hearts of old mice (B), or kidneys of old mice (C). Data is expressed as mean±SE and analyzed by one-way ANOVA, n=4, *p<0.05, **p<0.01 vs adult mice; #p<0.05, ##p<0.01 vs old mice. Bars indicate the standard error of the mean (SEM).

### DETAILED DESCRIPTION

Before describing various embodiments of the present disclosure in detail, it is to be understood that this disclosure is not limited only to the specific parameters, verbiage, and description of the particularly exemplified systems, methods, and/or products that may vary from one embodiment to the next. Thus, while certain embodiments of the present disclosure will be described in detail, with reference to specific features (e.g., configurations, parameters, properties, steps, components, ingredients, members, elements, parts, and/or portions, etc.), the descriptions are illustrative and are not to be construed as limiting the scope of the present disclosure and/or the claimed invention. In addition, the terminology used herein is for the purpose of describing the embodiments, and is not necessarily intended to limit the scope of the present disclosure and/or the claimed invention.

Unless defined otherwise, all technical and scientific terms used herein have the same meaning as commonly understood by one of ordinary skill in the art to which the present disclosure pertains.

Various aspects of the present disclosure, including systems, methods, and/or products may be illustrated with reference to one or more embodiments, which are exemplary in nature. As used herein, the terms “embodiment” mean “serving as an example, instance, or illustration,” and should not necessarily be construed as preferred or advantageous over other aspects disclosed herein. In addition, reference to an “ embodiment” of the present disclosure or invention is intended to provide an illustrative example without limiting the scope of the invention, which is indicated by the appended claims.

As used in this specification and the appended claims, the singular forms “a,” “an” and “the” each contemplate, include, and specifically disclose both the singular and plural referents, unless the context clearly dictates otherwise. For example, reference to a “protein” contemplates and specifically discloses one, as well as a plurality of (e.g., two or more, three or more, etc.) proteins. Similarly, use of a plural referent does not necessarily require a plurality of such referents, but contemplates, includes, specifically discloses, and/or provides support for a single, as well as a plurality of such referents, unless the context clearly dictates otherwise.

As used throughout this disclosure, the words “can” and “may” are used in a permissive sense (i.e., meaning having the potential to), rather than the mandatory sense (i.e., meaning must). Additionally, the terms “including,” “having,” “involving,” “containing,” “characterized by,” variants thereof (e.g., “includes,” “has,” and “involves,” “contains,” etc.), and similar terms as used herein, including the claims, shall be inclusive and/or open-ended, shall have the same meaning as the word “comprising” and variants thereof (e.g., “comprise” and “comprises”), and do not exclude additional, un-recited elements or method steps, illustratively.

The term “condition” refers to any disorder, disease, injury, or illness, as understood by those skilled in the art, that is manifested or anticipated in a patient. Manifestation of such a condition can be an early, middle, or late stage manifestation, as known in the art, including pre-condition symptoms, signs, or markers. Anticipation of such a condition can be or include the predicted, expected, envisioned, presumed, supposed, and/or speculated occurrence of the same, whether founded in scientific or medical evidence, risk assessment, or mere apprehension or trepidation.

The term “patient,” as used herein, is synonymous with the term “subject” and generally refers to any animal under the care of a medical professional, as that term is defined herein, with particular reference to (i) humans (under the care of a doctor, nurse, or medical assistant or volunteer) and (ii) non-human animals, such as non-human mammals (under the care of a veterinarian or other veterinary professional, assistant, or volunteer).

Embodiments of the present disclosure are also meant to encompass all pharmaceutically acceptable compounds according to Formula I that are isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C , ¹⁴C , ¹³N , ¹⁵N , ¹⁵O, ¹⁷O, ¹⁸O, ³¹P, ³²P, ³⁵S, ¹⁸F, ³⁶Cl, ¹²³I, and ¹²⁵I, respectively. These radiolabeled compounds may be useful to help determine or measure the effectiveness of the compounds, by characterizing, for example, the site or mode of action, or binding affinity to pharmacologically important site of action. Certain isotopically-labelled compounds according to Formula I, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula I can generally be prepared by conventional techniques known to those skilled in the art or by processes analogous to those described in the Preparations and Examples as set out below using an appropriate isotopically-labeled reagent in place of the non-labeled reagent previously employed.

Embodiments of the present disclosure may also encompass the in vivo metabolic products of the disclosed compounds. Such products may result from, for example, the oxidation, reduction, hydrolysis, amidation, esterification, and the like of the administered compound, primarily due to enzymatic processes. Accordingly, embodiments of the present disclosure include compounds produced by a process comprising administering a compound of this present disclosure to a mammal for a period of time sufficient to yield a metabolic product thereof. Such products are typically identified by administering a radiolabeled compound of the present disclosure in a detectable dose to an animal, such as rat, mouse, guinea pig, monkey, or to human, allowing sufficient time for metabolism to occur, and isolating its conversion products from the urine, blood or other biological samples.

The term(s) “carrier, diluent and/or excipient,” as well as “pharmaceutically acceptable carrier, diluent and/or excipient” include, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The term “salt” or “pharmaceutically acceptable salt” includes both acid and base addition salts.

Salts may include “acid addition salt” or “pharmaceutically acceptable acid addition salt”, which refer to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

Salts may include “base addition salt” or “pharmaceutically acceptable base addition salt”, which refer to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the compound of the present disclosure. As used herein, the term “solvate” refers to an aggregate that comprises one or more molecules of a compound of the present disclosure with one or more molecules of solvent. The solvent may be water, in which case the solvate may be a hydrate. Alternatively, the solvent may be an organic solvent. Thus, embodiments of the compounds of the present disclosure may exist as a hydrate, including a monohydrate, dihydrate, hemihydrate, sesquihydrate, trihydrate, tetrahydrate and the like, as well as the corresponding solvated forms. Embodiments of the compound of the present disclosure may be true solvates, while in other cases, the compound of the present disclosure may merely retain adventitious water or be a mixture of water plus some adventitious solvent.

A “pharmaceutical composition” refers to a formulation of a compound of the present disclosure and a medium generally accepted in the art for the delivery of the biologically active compound to mammals, e.g., humans. Such a medium includes all pharmaceutically acceptable carriers, diluents or excipients therefor.

“Mammal” includes humans and both domestic animals such as laboratory animals and household pets (e.g., cats, dogs, swine, cattle, sheep, goats, horses, rabbits), and non-domestic animals such as wildlife and the like.

“Effective amount” or “therapeutically effective amount” refers to that amount of a compound of the present disclosure which, when administered to a mammal, preferably a human, is sufficient to effect treatment, as defined below, of a disease associated with overexpression of a cyclin-dependent kinase (CDK) in the mammal, preferably a human. The amount of a compound of the present disclosure which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest in a mammal, preferably a human, having the disease or condition of interest, and includes:
(i) preventing the disease or condition from occurring in a mammal, in particular, when such mammal is predisposed to the condition but has not yet been diagnosed as having it;
(ii) inhibiting the disease or condition, i.e., arresting its development;
(iii) relieving the disease or condition, i.e., causing regression of the disease or condition; or
(iv) relieving the symptoms resulting from the disease or condition, i.e., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

Compounds of the present disclosure, or their pharmaceutically acceptable salts may contain one or more asymmetric centers and may thus give rise to enantiomers, diastereomers, and other stereoisomeric forms that may be defined, in terms of absolute stereochemistry, as (R)− or (S)− or, as (D)− or (L)− for amino acids. The present disclosure is meant to include all such possible isomers, as well as their racemic and optically pure forms. Optically active (+) and (−), (R)− and (S)−, or (D)− and (L)− isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. Embodiments of the present disclosure contemplate various stereoisomers and mixtures thereof and includes “enantiomers”, which refers to two stereoisomers whose molecules are nonsuperimposeable minor images of one another.

A “tautomer” refers to a proton shift from one atom of a molecule to another atom of the same molecule. Embodiments of the present disclosure include tautomers of any said compounds.

For the sake of brevity, the present disclosure may recite a list or range of numerical values. It will be appreciated, however, that where such a list or range of numerical values (e.g., greater than, less than, up to, at least, and/or about a certain value, and/or between two recited values) is disclosed or recited, any specific value or range of values falling within the disclosed values or list or range of values is likewise specifically disclosed and contemplated herein.

To facilitate understanding, like references (i.e., like naming of components and/or elements) have been used, where possible, to designate like elements common to different embodiments of the present disclosure Similarly, like components, or components with like functions, will be provided with similar reference designations, where possible. Specific language will be used herein to describe the exemplary embodiments. Nevertheless it will be understood that no limitation of the scope of the disclosure is thereby intended. Rather, it is to be understood that the language used to describe the exemplary embodiments is illustrative only and is not to be construed as limiting the scope of the disclosure (unless such language is expressly described herein as essential).

While the detailed description is separated into sections, the section headers and contents within each section are for organizational purposes only and are not intended to be self-contained descriptions and embodiments or to limit the scope of the description or the claims. Rather, the contents of each section within the detailed description are intended to be read and understood as a collective whole, where elements of one section may pertain to and/or inform other sections. Accordingly, embodiments specifically disclosed within one section may also relate to and/or serve as additional and/or alternative embodiments in another section having the same and/or similar products, methods, and/or terminology. Compounds Illustrative embodiments of the present disclosure include a compound according to Formula I:

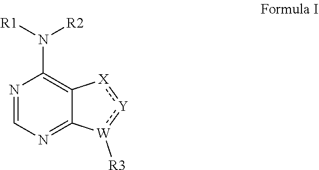

or a stereoisomer, tautomer or pharmaceutically acceptable salt thereof.

In various embodiments, X is (selected from (the group consisting of)) N, S, or C—R₄, wherein R₄ is (selected from (the group consisting of)) H or C1-C5 alkyl. In some embodiments, X is N. In some embodiments, X is S. In some embodiments, X is CH. In some embodiments, X is C1-C5 alkyl.

In various embodiments, Y is (selected from (the group consisting of)) N or C—R₄, wherein R₄ is (selected from (the group consisting of)) H or C1-C5 alkyl. In some embodiments, Y is N. In some embodiments, Y is CH. In some embodiments, Y is C1-C5 alkyl.

In various embodiments, the bond between X—Y is a single bond or a double bond. In some embodiments, the bond between X—Y is a single bond. In some embodiments, the bond between X-Y is a double bond.

In various embodiments, W is (selected from (the group consisting of)) N, S, or C. In some embodiments, W is N. In some embodiments, W is S. In some embodiments, W is C.

In various embodiments, the bond between Y—W is a single bond or a double bond. In some embodiments, the bond between Y—W is a single bond. In some embodiments, the bond between Y—W is a double bond.

In various embodiments, R₁ is (selected from (the group consisting of)) H, CH₃, or, together with R₂, forms substituted or unsubstituted heterocyclic amine. In some embodiments, R₁ is H. In some embodiments, R₁ is CH₃. In various embodiments, R₁, together with R₂, forms substituted or unsubstituted heterocyclic amine.

In various embodiments, R₂ is (selected from (the group consisting of)):

(CH₂)_{Z}—(CR₅R₆)_{V}—R₇, wherein Z is an integer from 0-2, V is an integer from 0-2, R₅ is (selected from (the group consisting of)) H or CH₃, R₆ is (selected from (the group consisting of)) H or CH₃; and R₇ is (selected from (the group consisting of)):

(i) saturated or unsaturated C3-C8 substituted or unsubstituted cycloalkyl or bicycloalkyl (e.g., bicyclo octane, preferably, bicyclo(2.2.2)octane), optionally substituted at one or more (ring) positions and each (ring) substituent is (selected from (the group consisting of)) halo, aryl (phenyl or benzyl), or branched or unbranched C1-C3 alkyl;

(ii) substituted or unsubstituted aryl (phenyl or benzyl), optionally substituted at one or more (1 or 2) (ring) positions and each (ring) substituent is (selected from (the group consisting of))halo or branched or unbranched C1-C3 alkyl; and/or

(iii) branched or unbranched C1-C3 alkyl (e.g., isopropyl);

C3-C7 substituted or unsubstituted cycloalkyl (e.g., 2-phenylcyclopropyl), optionally substituted at one or more (ring) positions and each (ring) substituent is (selected from (the group consisting of)) substituted or unsubstituted aryl (phenyl or benzyl), branched or unbranched C1-C3 substituted or unsubstituted alkyl; and/or

together with R₁, forms substituted or unsubstituted heterocyclic amine.

In some embodiments, R₂ is not H. In some embodiments, R₂ is not CH₃. In some embodiments, R₂ is not, one or more of, alkyl, ethyl, propyl, isopropyl, or butyl. In some embodiments, R₂ is not aryl, phenyl or benzyl. In some embodiments, R₂ is not carboxyl (COOH). In some embodiments, R₂ is not, one or more of, methanol, ethanol, propanol, or isopropanol.

In various embodiments, R₃ is (selected from (the group consisting of)): nothing, H, alkyl, cycloalkyl, aryl (phenyl or benzyl), nitrile, (CH₂)_{Z}CN, wherein Z is an integer from 1-3, branched or unbranched C1-C3 substituted or unsubstituted alkyl, C3-C7 substituted or unsubstituted cycloalkyl, aryl (phenyl or benzyl) optionally substituted at one or more (ring) positions and each (ring) substituent is (selected from (the group consisting of)) branched or unbranched C1-C3 substituted or unsubstituted alkyl, halo, or nitrile. In some embodiments, R₃ is nothing. In some embodiments, R₃ is H. In some embodiments, R₃ is alkyl. In some embodiments, R₃ is cycloalkyl. In some embodiments, R₃ is aryl (phenyl or benzyl). In some embodiments, R₃ is nitrile. In some embodiments, R₃ is (CH₂)_{Z}CN, wherein Z is an integer from 1-3. In some embodiments, R₃ is branched or unbranched C1-C3 substituted or unsubstituted alkyl. In some embodiments, R₃ is C3-C7 substituted or unsubstituted cycloalkyl. In some embodiments, R₃ is aryl (phenyl or benzyl) substituted at one or more (ring) positions and each (ring) substituent is (selected from (the group consisting of)) branched or unbranched C1-C3 substituted or unsubstituted alkyl, halo, or nitrile. In some embodiments, R₃ is aryl (phenyl or benzyl) substituted at one or more (ring) positions with branched or unbranched C1-C3 substituted or unsubstituted alkyl, halo, or nitrile.

In some embodiments, R₁ is H and R₂ is (CH₂)_{Z}—(CR₅R₆)_{V}—R₇, wherein Z is an integer from 1-2, V is 0, and R₇ is (selected from (the group consisting of):

saturated or unsaturated C4-C7 cycloalkyl, optionally substitute at one or more (ring) positions with one or more methyl, preferably saturated C4-C6 cycloalkyl, optionally substitute at one or more (ring) positions with one or more methyl, more preferably unsubstituted saturated C4-C6 cycloalkyl or saturated C6 cycloalkyl optionally substituted at one or more (ring) position with one or more methyl or fluoro, still more preferably monounsaturated C5-C7 cycloalkyl, preferably 2-(1-cycloalkenyl or 4-(1-cycloalkenyl, more preferably 2-(1-cyclopentenyl), 2-(1-cyclohexenyl), 2-(1-cycloheptenyl), 4-(1-cyclopentenyl), 4-(1-cyclohexenyl), or 4-(1-cycloheptenyl);

bicycloalkyl, preferably bicyclo octane, more preferably, bicyclo(2.2.2)octane;

aryl (or phenyl or benzyl), preferably substituted at one or more (ring) positions with halo, preferably chloro, more preferably 3-chlorobenzyl or 2,3-dichlorobenzyl;

isopropyl; and/or

C3 cycloalkyl, preferably substituted with aryl (or phenyl or benzyl), preferably 2-phenylcyclopropyl.

In some embodiments, R₁ is H and R₂ is (CH₂)_{Z}—(CR₅R₆)_{V}—R₇, wherein Z is 1, V is 1, R₅ is CH₃, R₆ is CH₃, and R₇ is monounsaturated cyclohexyl, preferably 2-(1-cyclohexenyl).

In some embodiments, R₁ is CH₃ and R₂ is (CH₂)_{Z}—(CR₅R₆)_{V}—R₇, Z is 2, V is 0, and R₇ is cyclohexyl.

In some embodiments, R₁ together with R₂, forms substituted or unsubstituted heterocyclic amine, preferably, substituted azepane, more preferably 4,4-diethylazepane. In some embodiments, R₁ and R₂ form a substituted or unsubstituted, saturated or unsaturated heterocyclic amine, preferably a substituted azepane, aziridine, azetidine, pyrrolidine, piperidine, or azocane, more preferably azepane, still more preferably 4,4-diethyl azepane.

In some embodiments:

X is N, Y is C—R₄, R₄ is H, W is N, the bond between X—Y is a double bond, and the bond between Y—W is a single bond, and wherein R₃ is preferably alkyl or cycloalkyl, more preferably ethyl or cyclopropyl;

X is C—R₄, Y is C—R₄, R₄ is H, W is N, the bond between X—Y is a double bond, and the bond between Y—W is a single bond, and preferably, wherein R₃ is H;

X is C—R₄, R₄ is H, Y is N, W is N, the bond between X—Y is a double bond, and the bond between Y—W is a single bond, and preferably, wherein R₃ is preferably (i) alkyl, more preferably methyl, (ii) substituted or unsubstituted aryl, phenyl or benzyl, still more preferably fluorophenyl or fluorobenzyl, or (iii) nitrile, preferably ethanenitrile or ethyl cyanide;

X is N or C—R₄, Y is C—R₄, each R₄ is, independently, H or CH₃, W is S, the bond between X—Y is a double bond, and the bond between Y—W is a single bond, and wherein R₃ is nothing;

X is C—R₄, Y is C—R₄, each R₄ is, independently, H or CH₃, W is S, the bond between X-Y is a double bond, and the bond between Y—W is a single bond, and wherein R₃ is nothing;

X is N, Y is C—R₄, R₄ is H, W is S, the bond between X—Y is a double bond, and the bond between Y—W is a single bond, and wherein R₃ is nothing;

X is N, Y is C—R₄, R₄ is H, W is N, the bond between X—Y is a double bond, and the bond between Y—W is a single bond, and wherein R₃ is preferably C2-C3 alkyl or cycloalkyl, preferably, ethyl or cyclopropyl; or

X is S, Y is C—R₄, R₄ is H, W is C, the bond between X—Y is a single bond, and the bond between Y—W is a double bond, and wherein R₃ is preferably H.

In some embodiments, X is N, Y is C—R₄, R₄ is H, W is N, the bond between X—Y is a double bond, and the bond between Y—W is a single bond, and wherein R₃ is preferably alkyl or cycloalkyl, more preferably ethyl or cyclopropyl.

In some embodiments, X is C—R₄, Y is C—R₄, R₄ is H, W is N, the bond between X—Y is a double bond, and the bond between Y—W is a single bond, and preferably, wherein R₃ is H.

In some embodiments, X is C—R₄, R₄ is H, Y is N, W is N, the bond between X—Y is a double bond, and the bond between Y—W is a single bond, and preferably, wherein R₃ is preferably (selected from (the group consisting of)) (i) alkyl, more preferably methyl, (ii) substituted or unsubstituted aryl, phenyl or benzyl, still more preferably fluorophenyl or fluorobenzyl, or (iii) nitrile, preferably ethanenitrile or ethyl cyanide.

In some embodiments, X is N or C—R₄, Y is C—R₄, each R₄ is, independently, H or CH₃, W is S, the bond between X—Y is a double bond, and the bond between Y—W is a single bond, and wherein R₃ is nothing.

In some embodiments, X is C—R₄, Y is C—R₄, each R₄ is, independently, H or CH₃, W is S, the bond between X—Y is a double bond, and the bond between Y—W is a single bond, and wherein R₃ is nothing.

In some embodiments, X is N, Y is C—R₄, R₄ is H, W is S, the bond between X—Y is a double bond, and the bond between Y—W is a single bond, and wherein R₃ is nothing.

In some embodiments, X is N, Y is C—R₄, R₄ is H, W is N, the bond between X—Y is a double bond, and the bond between Y—W is a single bond, and wherein R₃ is preferably (selected from (the group consisting of)) C2-C3 alkyl or cycloalkyl, preferably, ethyl or cyclopropyl.

In some embodiments, X is S, Y is C—R₄, R₄ is H, W is C, the bond between X—Y is a single bond, and the bond between Y—W is a double bond, and wherein R₃ is preferably H.

In some embodiments, X is N, Y is C—R₄, R₄ is H, W is N, the bond between X—Y is a double bond, the bond between Y—W is a single bond, R₁ is H, R₂ is (CH₂)_{Z}—(CR₅R₆)_{V}—R₇, wherein Z is 1, V is 0, and R₇ is (selected from (the group consisting of)) unsubstituted monounsaturated C6 cycloalkyl or unsubstituted cyclohexenyl, preferably 4-(1-cyclohexenyl), more preferably R-4-(1-cyclohexenyl) or S-4-(1-cyclohexenyl), and R₃ is cyclopropyl.

In some embodiments, X is N, Y is C—R₄, R₄ is H, W is N, the bond between X—Y is a double bond, and the bond between Y—W is a single bond, R₃ is (selected from (the group consisting of)) alkyl or cycloalkyl, preferably ethyl or cyclopropyl, R₁ is H and R₂ is (CH₂)_{Z}—(CR₅R₆)_{V}—R₇, wherein Z is an integer from 1-2, V is 0, and R₇ is (selected from (the group consisting of)):

unsubstituted saturated C4-C6 cycloalkyl;

saturated C6 cycloalkyl substituted at one (ring) positions with fluoro, methyl, or dimethyl, preferably 1-fluorocyclohexyl, 1-methylcyclohexyl, or 4,4-dimethylcyclohexyl;

unsubstituted monounsaturated C5-C7 cycloalkyl, preferably 2-(1-cycloalkenyl or 4-(1-cycloalkenyl, more preferably 2-(1-cyclopentenyl), 2-(1-cyclohexenyl), 2-(1-cycloheptenyl), 4-(1-cyclopentenyl), 4-(1-cyclohexenyl), or 4-(1-cycloheptenyl);

bicyclo octane, more preferably, bicyclo(2.2.2)octane;

aryl (or phenyl or benzyl) substituted at one or two (ring) positions with halo, preferably chloro, more preferably 3-chlorobenzyl or 2,3-dichlorobenzyl;

isopropyl; and/or

C3 cycloalkyl substituted with aryl (or phenyl or benzyl), preferably 2-phenylcyclopropyl.

Illustrative embodiments of the present disclosure include a compound according to Formula Ia:

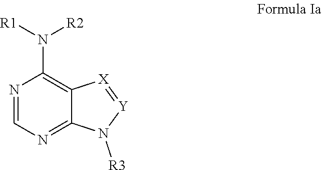

or a stereoisomer, tautomer or pharmaceutically acceptable salt thereof, wherein X, Y, R₁, R₂, and R₃ are as described for Formula I.

Illustrative embodiments of the present disclosure include a compound according to Formula II:

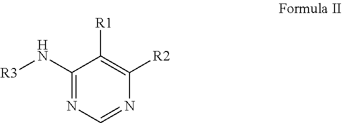

or a stereoisomer, tautomer or pharmaceutically acceptable salt thereof.

In various embodiments, R₁ is (selected from (the group consisting of) H or NH₂. In some embodiments, R₁ is H. In some embodiments, R₁ is NH₂.

In various embodiments, R₂ is (selected from (the group consisting of) saturated or unsaturated, heterocyclic amine or heterocyclic diamine. In some embodiments, R₂ is saturated or unsaturated heterocyclic amine. In some embodiments, R₂ is saturated or unsaturated heterocyclic diamine.

In some embodiments, R₂ is saturated heterocyclic amine, preferably N-pyrrolidine or N-piperidine, or unsaturated heterocyclic diamine, preferably N-pyrazole.

In some embodiments, when R₁ is H, then R₂ is N-pyrrolidine or N-piperidine.

In some embodiments, when R₁ is NH₂, then R₂ is N-pyrazole.

In various embodiments, R₃ is (CH₂)₂-R₇, wherein R₇ is unsaturated cycloalkyl.

In some embodiments, R₇ is monounsaturated C6-C7 cycloalkyl, preferably cyclohexenyl, more preferably 2-(1-cyclohexenyl), or cycloheptenyl, more preferably 2-(1-cycloheptenyl).

In some embodiments, the compound of Formula II is selected from the group consisting of Formulas IIa-IId:

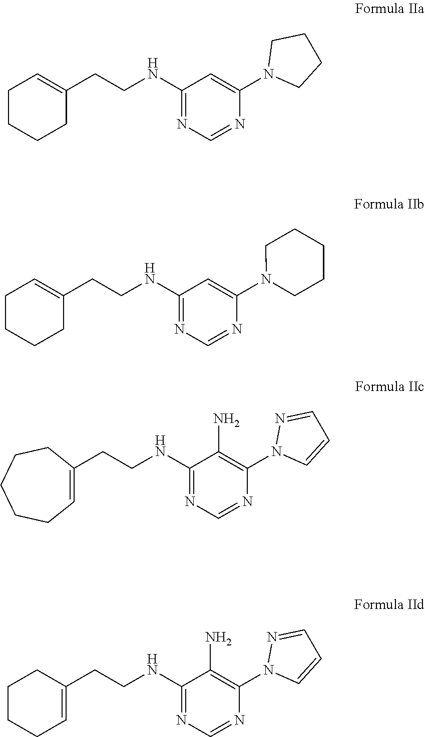 [SMILES: C1=C(CCNc2cc(N3CCCC3)ncn2)CCCC1 ; C1=C(CCNc2cc(N3CCCCC3)ncn2)CCCC1 ; Nc1c(NCCC2=CCCCCC2)ncnc1-n1cccn1 ; Nc1c(NCCC2=CCCCC2)ncnc1-n1cccn1]

or a stereoisomer, tautomer or pharmaceutically acceptable salt thereof.

Illustrative embodiments of the present disclosure include a compound according to Formula III:

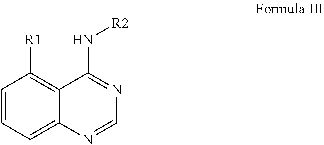

or a stereoisomer, tautomer or pharmaceutically acceptable salt thereof, wherein R₁ is halo; and R₂ is (CH₂)₂-R₇, wherein R₇ is unsaturated cycloalkyl. In some embodiments, R₁ is fluoro and R₇ is unsaturated cycloalkyl, preferably monounsaturated cycloalkyl, more preferably cyclohexenyl, more preferably 2-(1-cyclohexenyl), still most preferably, wherein the compound is according to Formula IIIa:

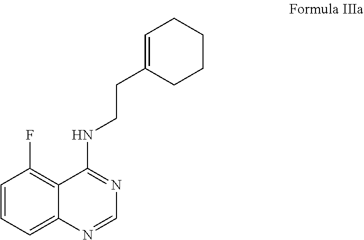 [SMILES: Fc1cccc2ncnc(NCCC3=CCCCC3)c12]

In some embodiments, the compound according to Formula I can be one of Compounds 1-49 of Table 1.

### Compositions and Medicaments

Administration of the compounds of the present disclosure, or their pharmaceutically acceptable salts, in pure form or in an appropriate pharmaceutical composition or medicament, can be carried out via any of the accepted modes of administration of agents for serving similar utilities. The pharmaceutical compositions or medicaments of embodiments of the present disclosure can be prepared by combining a compound of the present disclosure with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions or medicaments include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Pharmaceutical compositions or medicaments of the present disclosure are formulated so as to allow the active ingredients contained therein to be bioavailable upon administration of the composition or medicament to a patient. Compositions or medicaments that will be administered to a subject or patient take the form of one or more dosage units, where for example, a tablet may be a single dosage unit, and a container of a compound of the present disclosure in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition or medicament to be administered will, in any event, contain a therapeutically effective amount of a compound of the present disclosure, or a pharmaceutically acceptable salt thereof, for treatment of a disease or condition of interest in accordance with the teachings of this disclosure.

A pharmaceutical composition or medicament of some embodiments of the present disclosure may be in the form of a solid or liquid. In one aspect, the carrier(s) are particulate, so that the compositions or medicaments are, for example, in tablet or powder form. The carrier(s) may be liquid, with the compositions being, for example, an oral syrup, injectable liquid or an aerosol, which is useful in, for example, inhalation or inhalatory administration.

When intended for oral administration, the pharmaceutical composition or medicament is preferably in either solid or liquid form, where semi-solid, semi-liquid, suspension and gel forms are included within the forms considered herein as either solid or liquid.

As a solid composition or medicament for oral administration, the pharmaceutical composition or medicament may be formulated into a powder, granule, compressed tablet, pill, capsule, chewing gum, wafer or the like form. Such a solid composition or medicament will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition or medicament is in the form of a capsule, for example, a gelatin capsule, it may contain, in addition to materials of the above type, a liquid carrier such as polyethylene glycol or oil.

The pharmaceutical composition or medicament may be in the form of a liquid, for example, an elixir, syrup, solution, emulsion or suspension. The liquid may be for oral administration or for delivery by injection, as two examples. When intended for oral administration, preferred composition or medicament contain, in addition to the present compounds, one or more of a sweetening agent, preservatives, dye/colorant and flavor enhancer. In a composition or medicament intended to be administered by injection, one or more of a surfactant, preservative, wetting agent, dispersing agent, suspending agent, buffer, stabilizer and isotonic agent may be included.

The liquid pharmaceutical compositions and medicaments of some embodiments of the present disclosure, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition or medicament is preferably sterile.

A liquid pharmaceutical composition or medicament of certain embodiments of the present disclosure intended for either parenteral or oral administration should contain an amount of a compound of the present disclosure such that a suitable dosage will be obtained.

In some embodiments, the pharmaceutical composition or medicament of the present disclosure may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition or medicament for topical administration. If intended for transdermal administration, the composition or medicament may include a transdermal patch or iontophoresis device.

The pharmaceutical composition or medicament of various embodiments of the present disclosure may be intended for rectal administration, in the form, for example, of a suppository, which will melt in the rectum and release the drug. The composition or medicament for rectal administration may contain an oleaginous base as a suitable nonirritating excipient. Such bases include, without limitation, lanolin, cocoa butter and polyethylene glycol.

Embodiments of the pharmaceutical composition or medicament of the present disclosure may include various materials, which modify the physical form of a solid or liquid dosage unit. For example, the composition or medicament may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition or medicament of some embodiments of the present disclosure in solid or liquid form may include an agent that binds to the compound of the present disclosure and thereby assists in the delivery of the compound. Suitable agents that may act in this capacity include a monoclonal or polyclonal antibody, a protein or a liposome.

The pharmaceutical composition or medicament of other embodiments of the present disclosure may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the present disclosure may be delivered in single phase, bi-phasic, or tri-phasic systems in order to deliver the active ingredient(s).

Delivery of the aerosol includes the necessary container, activators, valves, subcontainers, and the like, which together may form a kit. One skilled in the art, without undue experimentation may determine preferred aerosols.

In some embodiments, the pharmaceutical compositions or medicaments of the present disclosure may be prepared by methodology well known in the pharmaceutical art. For example, a pharmaceutical composition or medicament intended to be administered by injection can be prepared by combining a compound of the present disclosure with sterile, distilled water so as to form a solution. A surfactant may be added to facilitate the formation of a homogeneous solution or suspension. Surfactants are compounds that non-covalently interact with the compound of the present disclosure so as to facilitate dissolution or homogeneous suspension of the compound in the aqueous delivery system.

The compounds of the present disclosure, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy.

Compounds of the present disclosure, or pharmaceutically acceptable derivatives thereof, may also be administered simultaneously with, prior to, or after administration of one or more other therapeutic agents. Such combination therapy includes administration of a single pharmaceutical dosage formulation which contains a compound of the present disclosure and one or more additional active agents, as well as administration of the compound of the present disclosure and each active agent in its own separate pharmaceutical dosage formulation. For example, a compound of the present disclosure and the other active agent can be administered to the patient together in a single oral dosage composition such as a tablet or capsule, or each agent administered in separate oral dosage formulations. Where separate dosage formulations are used, the compounds of the present disclosure and one or more additional active agents can be administered at essentially the same time, i.e., concurrently, or at separately staggered times, i.e., sequentially; combination therapy is understood to include all these regimens.

In some embodiments, the concentration of the compound of Formula I provided in the pharmaceutical compositions or medicaments of the present disclosure is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001%, w/w, w/v or v/v, of the pharmaceutical composition or medicament.

In some embodiments, the concentration of the compound of Formula I provided in the pharmaceutical compositions or medicaments of the present disclosure is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001%, w/w, w/v, or v/v, of the pharmaceutical composition or medicament.

In some embodiments, the concentration of the compound of the Formula I provided in the pharmaceutical compositions or medicaments of the present disclosure is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v, of the pharmaceutical composition or medicament.

In some embodiments, the concentration of the compound of Formula I provided in the pharmaceutical compositions or medicaments of the present disclosure is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v, of the pharmaceutical composition or medicament.

In some embodiments, the amount the compound of Formula I provided in the pharmaceutical compositions or medicaments of the present disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g of the pharmaceutical composition or medicament.

In some embodiments, the amount of the compound of Formula I provided in the pharmaceutical compositions or medicaments of the present disclosure is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g of the pharmaceutical composition or medicament.

In some embodiments, the amount of the compound of Formula I provided in the pharmaceutical compositions or medicaments of the present disclosure is in the range of 0.0001-10 g, 0.0005-9 g, 0.001-8 g, 0.005-7 g, 0.01-6 g, 0.05-5 g, 0.1-4 g, 0.5-4 g, or 1-3 g of the pharmaceutical composition or medicament.

### Chemical Synthesis

All chemicals, reagents and solvents were obtained from commercial vendors, such as Enamine, Sigma-Aldrich, and Fisher Scientific. Indicated reaction temperatures refer to those of the reaction bath, while room temperature (rt) is noted as 25° C. Analytical thin layer chromatography (TLC) was performed with glass backed silica plates (20×20 cm, pH=5, MF254). Visualization was accomplished using a 254 nm UV lamp. ¹H spectra were recorded on a 400 MHz spectrometer using solutions of samples in DMSO-d6 or other commercially-available deuterated solvents, as noted. Chemical shifts are reported in ppm with tetramethylsilane as standard. Data are reported as follows: chemical shift, number of protons, multiplicity (s=singlet, d=doublet, dd=doublet of doublet, t=triplet, q=quartet, b=broad, m=multiplet). All novel compounds were characterized by 1 H-NMR and mass spectroscopy (MS).

### Example 1

### N-[(cyclohex-3-en-1-yl)methyl]-9-cyclopropyl-9H-purin-6-amine

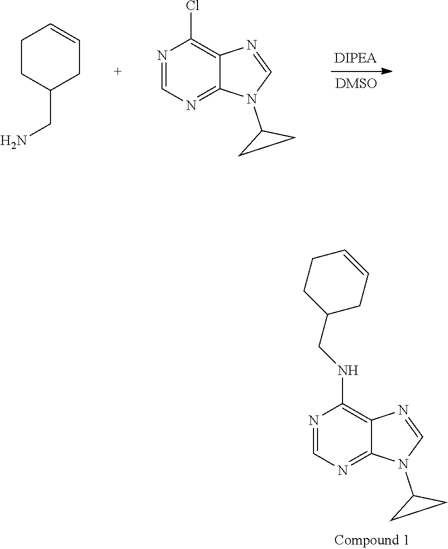 [SMILES: NCC1CC=CCC1 ; Clc1ncnc2c1ncn2C1CC1 ; C1=CCC(CNc2ncnc3c2ncn3C2CC2)CC1]

A stirred mixture of 1-(cyclohex-3-en-1-yl)methanamine (1.3 g, 11.7 mmol) and N,N-diisopropylethylamine (1.727 g, 13.4 mmol) in DMSO (15 mL) was stirred for 15 min at r.t. and 6-chloro-9-cyclopropyl-9H-purine (2.168 g, 11.1 mmol) was added. The reaction mixture was heated to 90° C. and stirred for 16 h. After cooling down, the resulting mixture was diluted with water and extracted with ethyl acetate. The organic layer was washed with water, dried over Na2SO4 and evaporated in vacuo at 50° C. to afford the crude residue, which was purified by flash column chromatography to obtain compound 1, N-[(cyclohex-3-en-1-yl)methyl]-9-cyclopropyl-9H-purin-6-amine (2.1 g, 70%). 1H NMR (400 MHz, DMSO-d6): δ8.18 (s, 1H), 8.08 (s, 1H), 7.77 (t, J=10.2 Hz, 1H), 5.62 (s, 2H), 3.91-3.47 (m, 1H), 3.48-3.36 (m, 2H), 2.20-1.82 (m, 4H), 1.81-1.61 (m, 2H), 1.27-1.12 (m, 1H), 1.14-0.91 (m, 4H). MS: m/z 270 [M+H]+.

### Example 2

### N-[2-(cyclohept-1-en-1-yl)ethyl]-7H-pyrrolo[2,3 -d]pyrimidin-4-amine

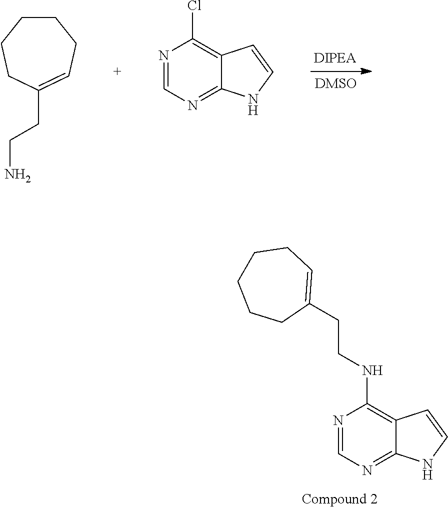 [SMILES: NCCC1=CCCCCC1 ; Clc1ncnc2[nH]ccc12 ; C1=C(CCNc2ncnc3[nH]ccc23)CCCCC1]

A stirred mixture of 2-(cyclohept-1-en-1-yl)ethan-1-amine (3.5 g, 12.7 mmol, hydrochloride salt) and N,N-diisopropylethylamine (3.328 g, 25.7 mmol) in DMSO (15 mL) was stirred for 15 min at r.t. and 4-chloro-7H-pyrrolo[2,3-d]pyrimidine (1.797 g, 11.7 mmol) was added. The reaction mixture was heated to 90° C. and stirred for 16 h. After cooling down, the resulting mixture was diluted with water and extracted with ethyl acetate. The organic layer was washed with water, dried over Na2SO4 and evaporated in vacuo at 50° C. to afford the crude residue, which was purified by flash column chromatography to obtain compound 2, N-[2-(cyclohept-1-en-1-yl)ethyl]-7H-pyrrolo[2,3-d]pyrimidin-4-amine (1.238 g, 41%). 1H NMR (400 MHz, DMSO-d6 δ11.44 (s, 1H), 8.08 (s, 1H), 7.27 (t, J=5.1 Hz, 1H), 7.03 (s, 1H), 6.50 (s, 1H), 5.58 (t, J=6.3 Hz, 1H), 3.54-3.44 (m, 2H), 2.25 (t, J=7.4 Hz, 2H), 2.18-2.08 (m, J=10.4 Hz, 2H), 2.05-1.90 (m, J=8.2 Hz, 2H), 1.75-1.57 (m, J=5.4 Hz, 2H), 1.52-1.21 (m, 4H). MS: m/z 259 [M+H]+.

### Example 3

### Additional Illustrative Compounds

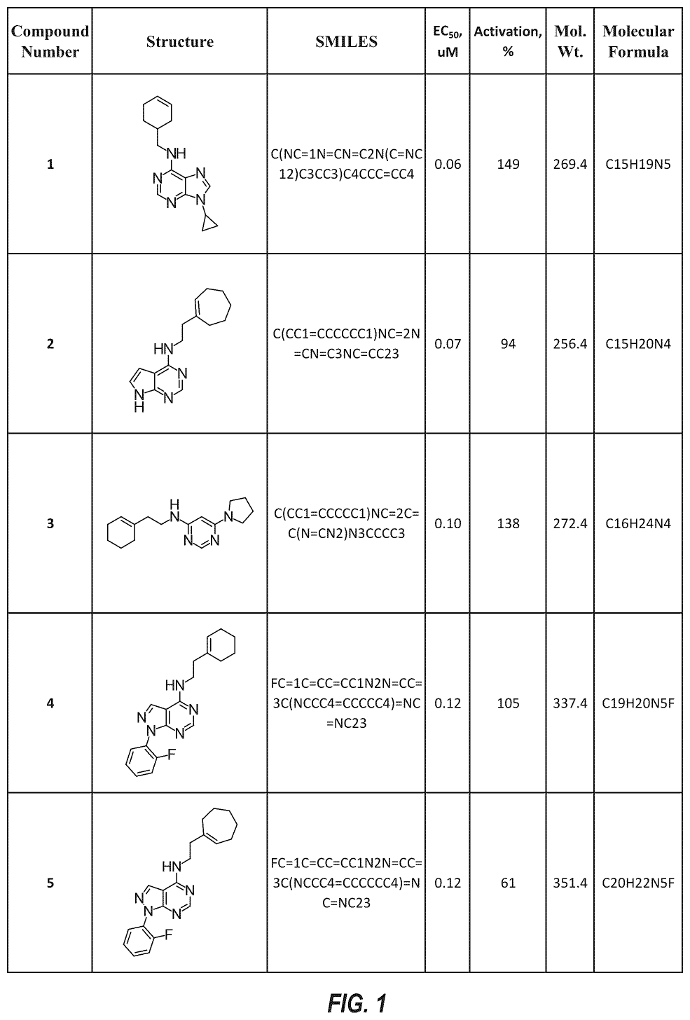

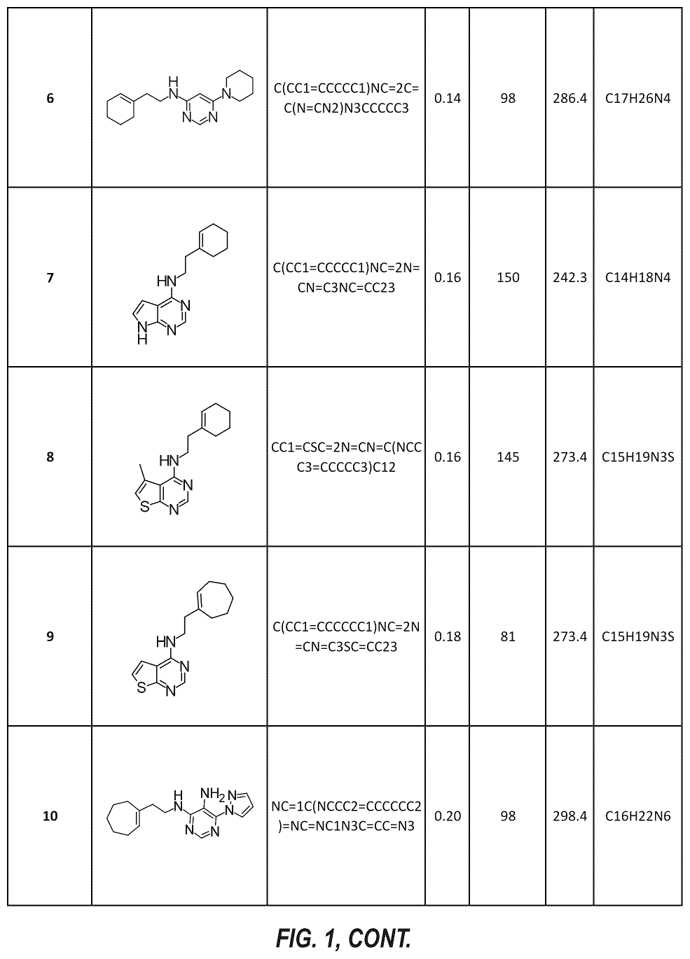

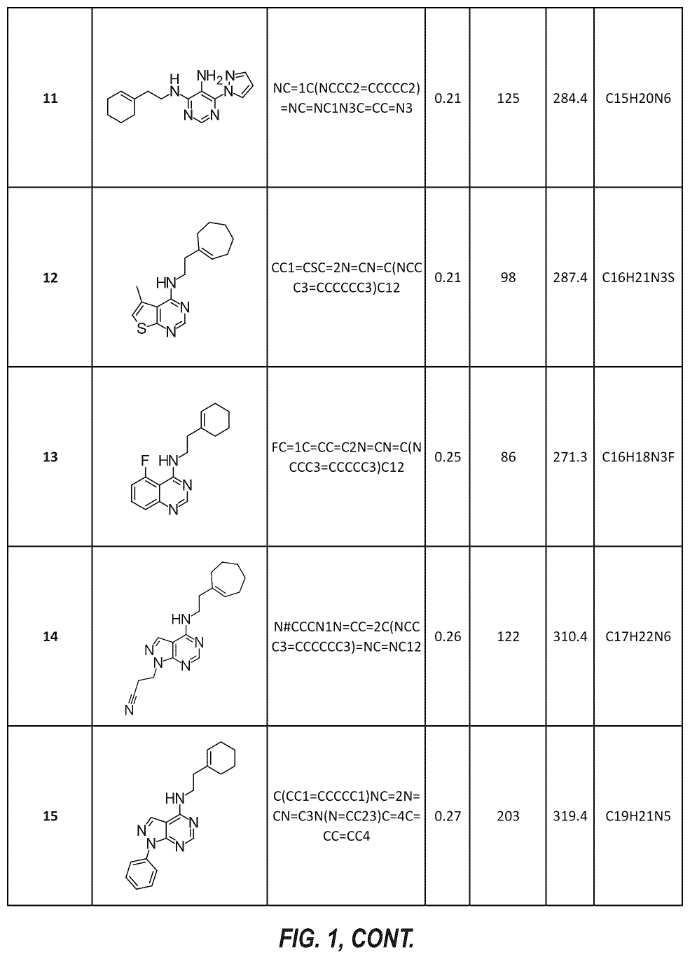

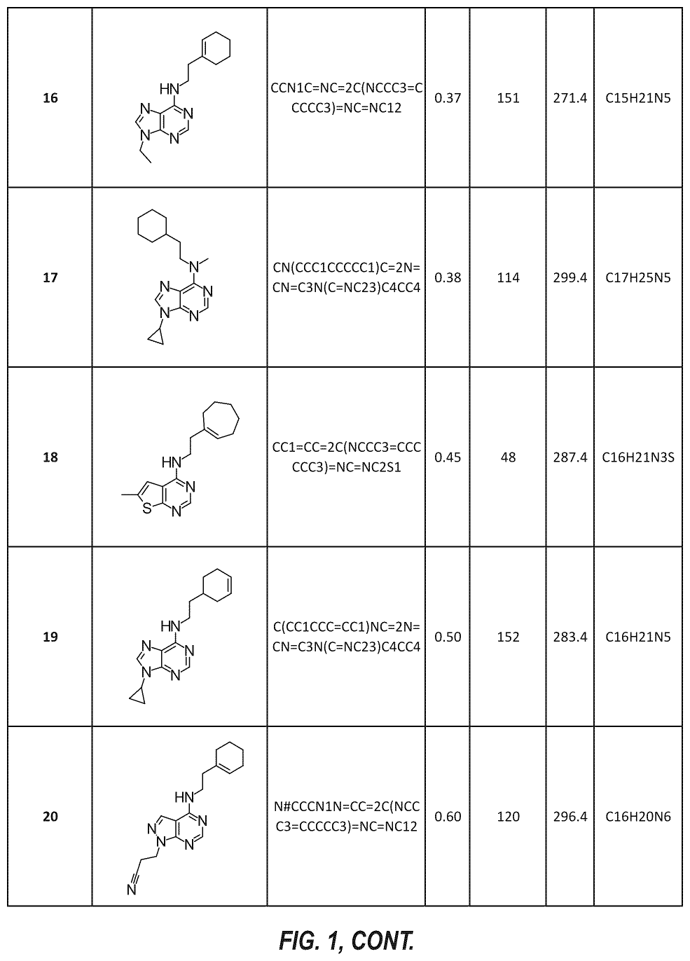

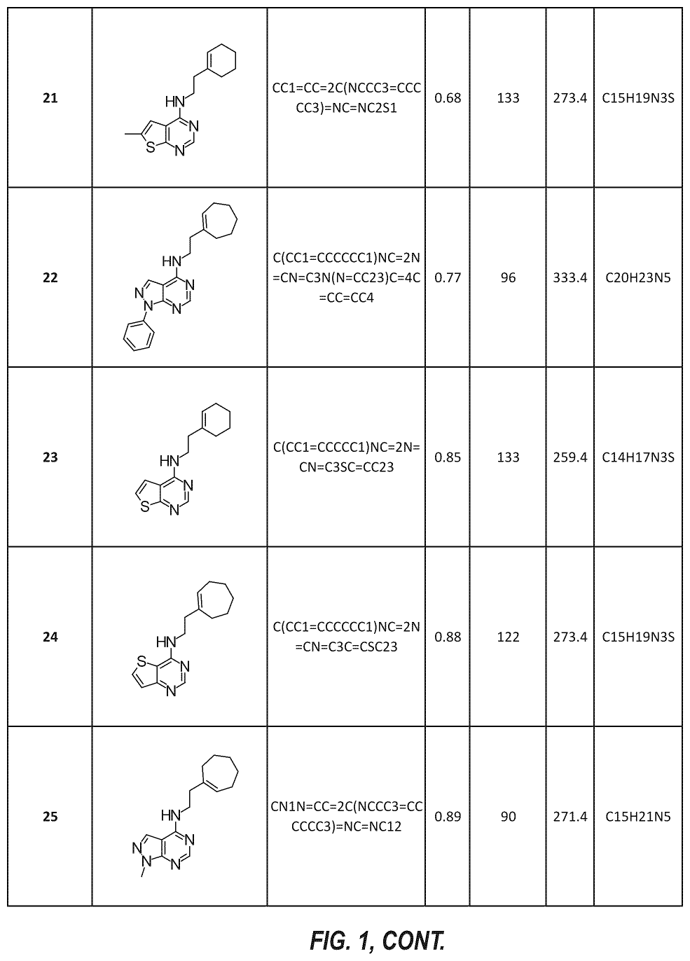

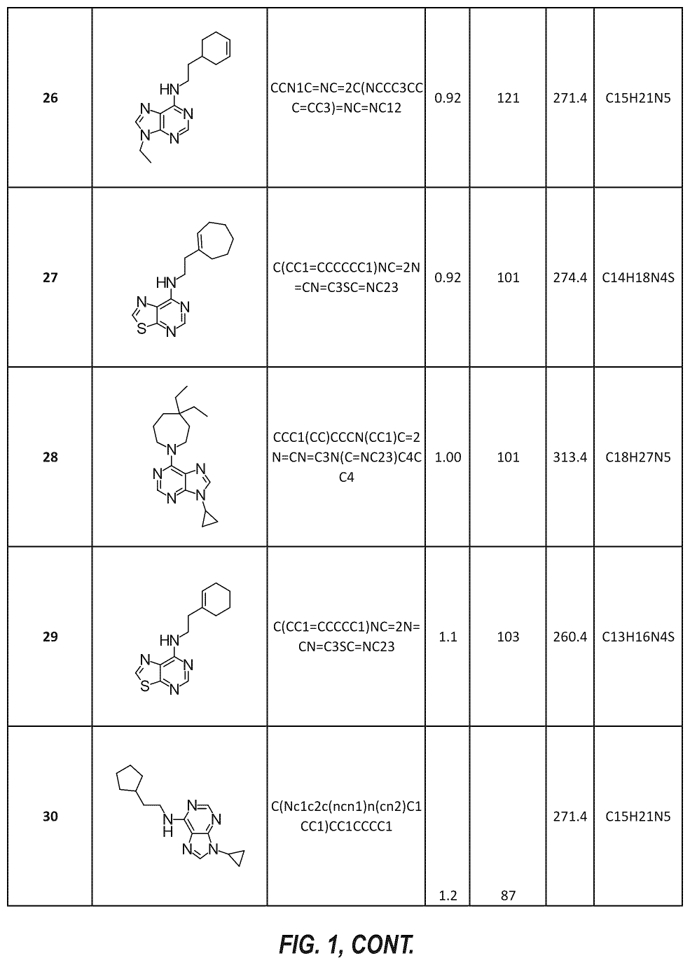

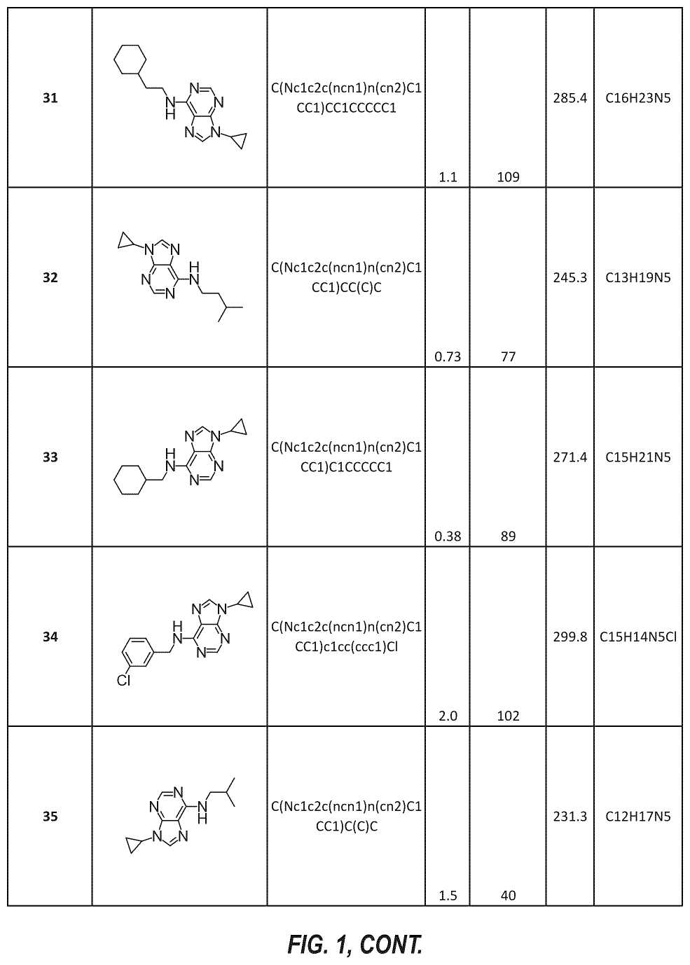

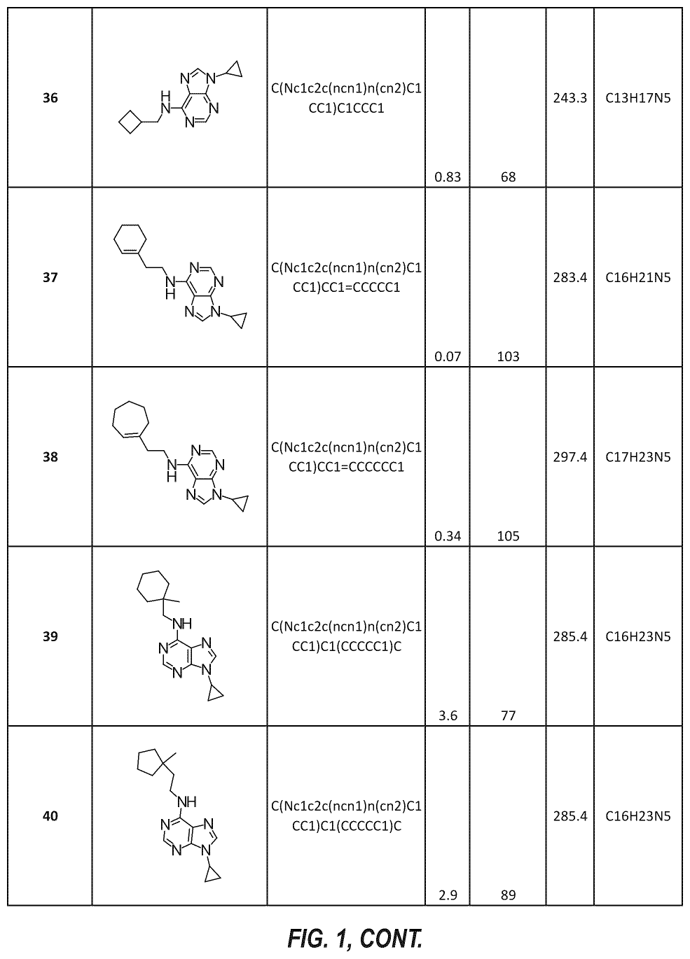

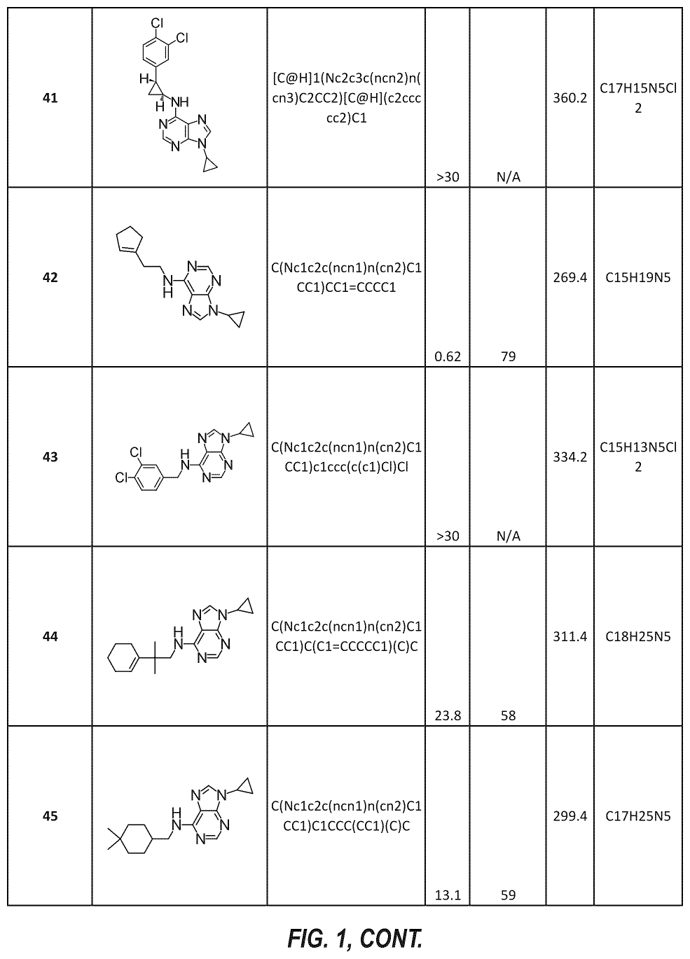

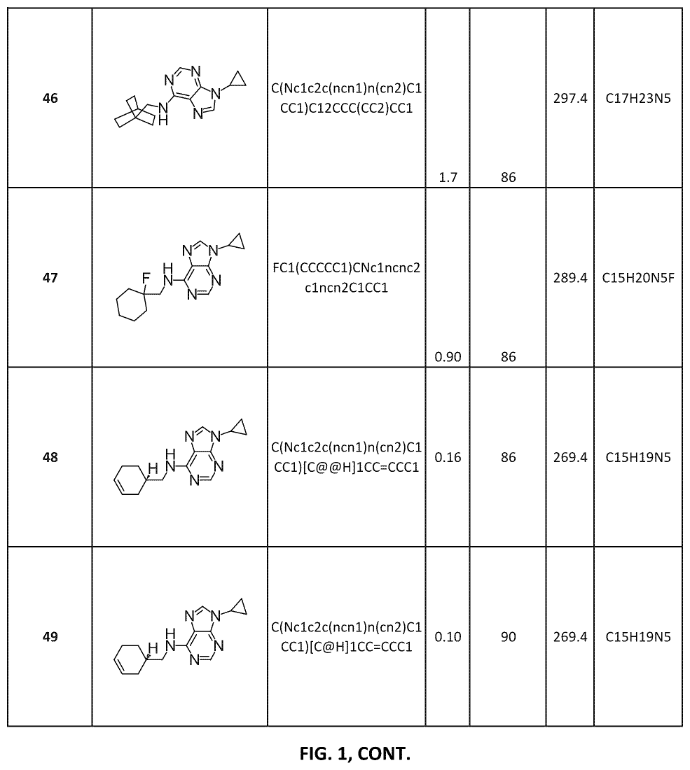

A listing of illustrative Compounds 1-49 are presented in FIG. 1. Compounds 3-49 are prepared and purified via similar methods to Examples 1 and 2, with the appropriate amine and heterocyclic reagents, and are characterized by 1H NMR, HPLC and MS.

### Assays and Experimentation

### Example 4.

### Table 2. Luciferase Reporter Assay to Evaluate Klotho Gene Expression Changes

HEK293 cells/pKlotho_Luc in suspension (20 μl/well, density 0.25×106 cells/ml) were dispensed into assay-ready 384-well tissue culture treated plates (plates (BD BioCoat Poly-D-Lys coated, Cat.354661) preloaded with 1000× stock of test compounds in DMSO (20 nL/well, 10 concentration points, 3× dilutions from 20 uM to 0.001 uM). Growth medium: DMEM, 1% FBS, Pen/Strep, 600 ug/ml G418. Negative control: DMSO. Positive control: 2 uM Compound H from King, et. al. Biochem. J. (2012) 441, 453-461. Cells were incubated in CO₂ incubator overnight. At the end of the incubation time, 20 ul/well of the luciferase assay solution (Steady-Luc Firefly HTS Assay Biotium, Cat 30028) were added to the plate and mixed on a VibraTranslator (Union Scientific). Plates were incubated for 5 min at 25° C. Steady glow luminescence signal was read using a BMG Labtech PHERAstar FSX reader.

TABLE 2

| Compound Number | EC₅₀, uM | Activation, % |
|---|---|---|
| 1 | 0.06 | 149 |
| 2 | 0.07 | 94 |
| 3 | 0.10 | 138 |
| 4 | 0.12 | 105 |
| 5 | 0.12 | 61 |
| 6 | 0.14 | 98 |
| 7 | 0.16 | 150 |
| 8 | 0.16 | 145 |
| 9 | 0.18 | 81 |
| 10 | 0.20 | 98 |
| 11 | 0.21 | 125 |
| 12 | 0.21 | 98 |
| 13 | 0.25 | 86 |
| 14 | 0.26 | 122 |
| 15 | 0.27 | 203 |
| 16 | 0.37 | 151 |
| 17 | 0.38 | 114 |
| 18 | 0.45 | 48 |
| 19 | 0.50 | 152 |
| 20 | 0.60 | 120 |
| 21 | 0.68 | 133 |
| 22 | 0.77 | 96 |
| 23 | 0.85 | 133 |
| 24 | 0.88 | 122 |
| 25 | 0.89 | 90 |
| 26 | 0.92 | 121 |
| 27 | 0.92 | 101 |
| 28 | 1.00 | 101 |
| 29 | 1.1 | 103 |
| 30 | 1.2 | 87 |
| 31 | 1.1 | 109 |
| 32 | 0.73 | 77 |
| 33 | 0.38 | 89 |
| 34 | 2.0 | 102 |
| 35 | 1.5 | 40 |
| 36 | 0.83 | 68 |
| 37 | 0.07 | 103 |
| 38 | 0.34 | 105 |
| 39 | 3.6 | 77 |
| 40 | 2.9 | 89 |
| 41 | >30 | N/A |
| 42 | 0.62 | 79 |
| 43 | >30 | N/A |
| 44 | 23.8 | 58 |
| 45 | 13.1 | 59 |
| 46 | 1.7 | 86 |
| 47 | 0.90 | 86 |
| 48 | 0.16 | 86 |
| 49 | 0.10 | 90 |

Accordingly, each of compounds 1-49 were shown to be effective at increasing klotho gene expression.

### Aged Rat Efficacy Model

The aim of the study was to assess the efficacy of test compound in improving physiologic parameters in aged rats such as blood pressure values. The experiment included daily repeated administrations of the test compound for 14 consecutive days and follow up observations within a 15-day post-dosing period. Body weight (BWt) were monitored daily during the dosing period and twice a week during the post-dosing period. Blood pressure (BP) was measured on 0, 7th, 14th, 21s t and 28th day of the study. Study design, animal selection, handling and treatment were all in accordance with the CROs efficacy study protocols and Standard Operation Procedure, and the Animal Care Guidelines.

Fifteen aged male Wistar rats (22.5 months old; initial body weights ranged from 474.8 g to 672.4 g with an average body weight across the groups of 540.8 g) were randomized into two groups by body weight and systolic blood pressure: 11 animals distributed to compound 49-treated group and 4 animals—to vehicle-treated group. Two rats (rat#8, rat#16) previously exposed to local surgery due to tumor appearing, did not undergo to randomizing process and were placed one into compound-treated group (rat#8) and another into vehicle-treated group (rat#16). Rats in the compound-treated group were repeatedly PO dosed with Compound 49 at the dose of 50 mg/kg at the volume of 5 ml/kg with an interval of 24 h for thirteen consecutive days starting from Day 1 of the experimental period. Day 14 animals were treated with Compound 49 at ⅓ of the dose. Experimental animals from vehicle-treated group were repeatedly dosed with vehicle (0.75% PVP K30 and 0.025% sodium docusate in distilled water) at the volume of 5 ml/kg with an interval of 24 h for thirteen consecutive days starting from Day 1 of the experimental period and at Day 14 they obtained ⅓ of the vehicle volume.

Blood pressure was measured by the Tail-Cuff Method using the Coda Non-invasive Blood-Pressure System. Individual body weights and relative changes in body weight (compared to the appropriate weight on Day 0) were recorded. Experimental animals' body weights were reduced during the administration period and gained during postdosing period in the same manner for Compound 49-treated and vehicle-treated groups.

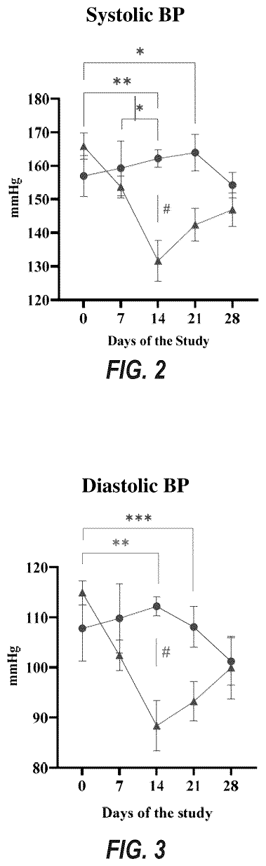

Systolic and diastolic blood pressure values were recorded, Significant difference between systolic blood pressure values in Compound 49-treated group and vehicle-treated group was registered at Day 14 of the study (see FIG. 2). Also, considerable decrease of systolic blood pressure was recorded during the Study in the Compound 49-treated group Diastolic blood pressure values were altered in the same manner: significant difference between the treated group and vehicle-treated group was pronounced on Day 14 (FIG. 3).

FIG. 2. Systolic BP values (mean per group±SE) in aged male Wistar rats during the study. Statistical differences calculated using Tukey's multiple comparisons test at the level of p<0.05, p<0.01 were given with asterisks * and ** within the Compound 49-treated group, and difference between the groups calculated using Sidak's multiple comparisons test at the level of p<0.05 were given with hash #, respectively. Green circles represent the vehicle-treated group and the red triangles are the drug-treated group.

FIG. 3: Diastolic BP values (mean per group±SE) in aged male Wistar rats during the study. Statistical differences calculated using Tukey's multiple comparisons test at the level of p<0.05, p<0.01 were given with asterisks * and ** within the Compound 49-treated group, and difference between the groups calculated using Sidak's multiple comparisons test at the level of p<0.05 were given with hash #, respectively. Green circles represent the vehicle-treated group and the red triangles are the drug-treated group.

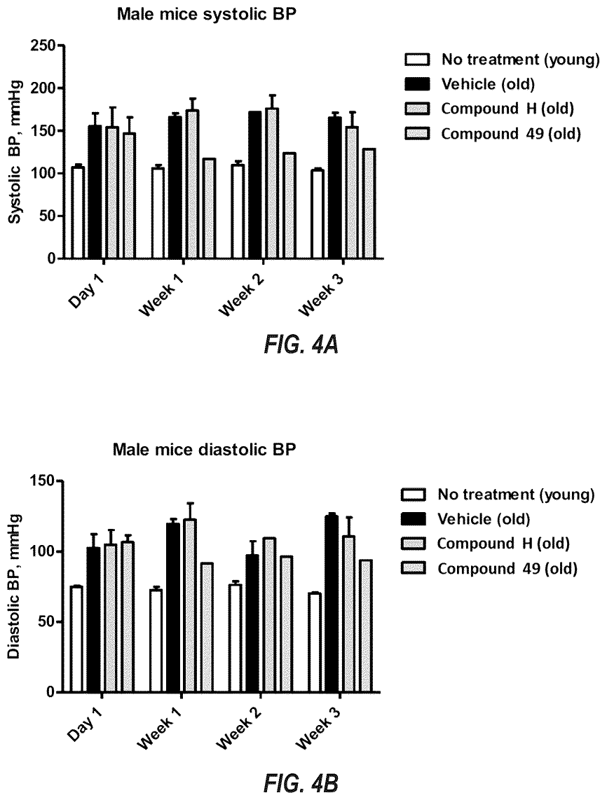

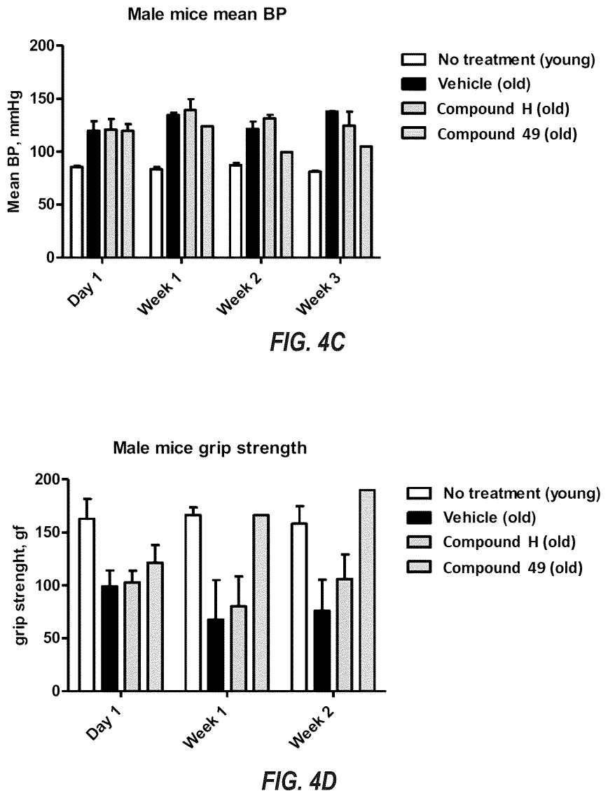

FIGS. 4A-4D illustrate, respectively systolic blood pressure (4A), diastolic blood pressure (4B), mean blood pressure (4C), and grip strength (4D) in male mice treated with illustrative Compound 49.

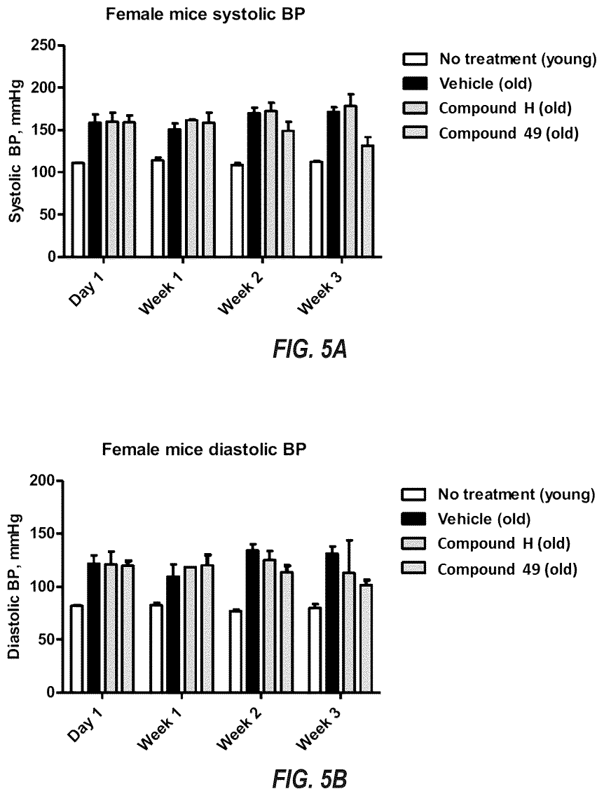

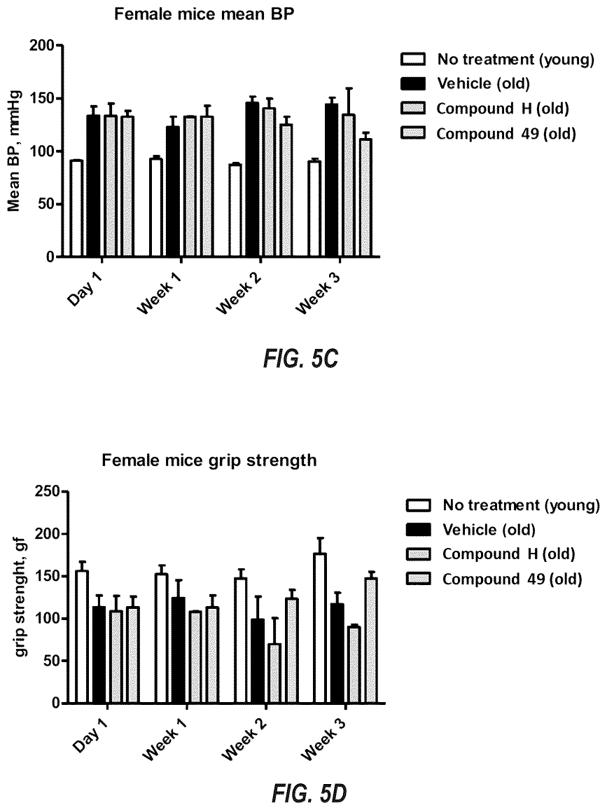

FIGS. 5A-5D illustrate, respectively systolic blood pressure (5A), diastolic blood pressure (5B), mean blood pressure (5C), and grip strength (5D) in female mice treated with illustrative Compound 49.

### Conclusion

While the foregoing detailed description makes reference to specific exemplary embodiments, the present disclosure may be embodied in other specific forms without departing from its spirit or essential characteristics. Accordingly, the described embodiments are to be considered in all respects only as illustrative and not restrictive. For instance, various substitutions, alterations, and/or modifications of the inventive features described and/or illustrated herein, and additional applications of the principles described and/or illustrated herein, which would occur to one skilled in the relevant art and having possession of this disclosure, can be made to the described and/or illustrated embodiments without departing from the spirit and scope of the disclosure as defined by the appended claims. Such substitutions, alterations, and/or modifications are to be considered within the scope of this disclosure.

The scope of the invention is, therefore, indicated by the appended claims rather than by the foregoing description. The limitations recited in the claims are to be interpreted broadly based on the language employed in the claims and not limited to specific examples described in the foregoing detailed description, which examples are to be construed as non-exclusive and non-exhaustive. All changes which come within the meaning and range of equivalency of the claims are to be embraced within their scope.

It will also be appreciated that various features of certain embodiments can be compatible with, combined with, included in, and/or incorporated into other embodiments of the present disclosure. For instance, systems, methods, and/or products according to certain embodiments of the present disclosure may include, incorporate, or otherwise comprise features described in other embodiments disclosed and/or described herein. Thus, disclosure of certain features relative to a specific embodiment of the present disclosure should not be construed as limiting application or inclusion of said features to the specific embodiment.

In addition, unless a feature is described as being requiring in a particular embodiment, features described in the various embodiments can be optional and may not be included in other embodiments of the present disclosure. Moreover, unless a feature is described as requiring another feature in combination therewith, any feature herein may be combined with any other feature of a same or different embodiment disclosed herein. It will be appreciated that while features may be optional in certain embodiments, when features are included in such embodiments, they can be required to have a specific configuration as described in the present disclosure.

Likewise, any steps recited in any method or process described herein and/or recited in the claims can be executed in any suitable order and are not necessarily limited to the order described and/or recited, unless otherwise stated (explicitly or implicitly). Such steps can, however, also be required to be performed in a specific order or any suitable order in certain embodiments of the present disclosure.

Furthermore, various well-known aspects of illustrative systems, methods, products, and the like are not described herein in particular detail in order to avoid obscuring aspects of the example embodiments. Such aspects are, however, also contemplated herein.

The present disclosure describes methods and compositions comprising N-phenyl-1H-indole-3-carboxamide derivates (also referred to herein as “active agents”), for example, N-halophen yl-1H-indole-3-carboxamides for the treatment of diseases and conditions such as, but not limited to, arterial stiffness, hypertension, and anti-aging effects, in a subject.

Before further describing various embodiments of the compounds, compositions and methods of the present disclosure in more detail by way of exemplary description, examples, and results, it is to be understood that the compounds, compositions, and methods of present disclosure are not limited in application to the details of specific embodiments and examples as set forth in the following description. The description provided herein is intended for purposes of illustration only and is not intended to be construed in a limiting sense. As such, the language used herein is intended to be given the broadest possible scope and meaning; and the embodiments and examples are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the present disclosure. However, it will be apparent to a person having ordinary skill in the art that the present disclosure may be practiced without these specific details. In other instances, features which are well known to persons of ordinary skill in the art have not been described in detail to avoid unnecessary complications of the description. It is intended that all alternatives, substitutions, modifications and equivalents apparent to those having ordinary skill in the art are included within the scope of the present disclosure.

All of the compounds, compositions, and methods of application and use thereof disclosed herein can be made and executed without undue experimentation in light of the present disclosure. Thus, while the compounds, compositions, and methods of the present disclosure have been described in terms of particular embodiments, it will be apparent to those of skill in the art that variations may be applied to the compounds, compositions and/or methods and in the steps or in the sequence of steps of the methods described herein without departing from the concept, spirit, and scope of the inventive concepts,

All patents, published patent applications, and non-patent publications mentioned in the specification or referenced in any portion of this application are herein expressly incorporated by reference in their entirety to the same extent as if each individual patent or publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

As utilized in accordance with the methods and compositions of the present disclosure, the following terms, unless otherwise indicated, shall be understood to have the follow meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or”' unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100 or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and. so forth. Reference to a range of 1-50 therefore includes 1, 2, 3, 4, 5, 6, 7, 8, 9, 10, 11, 12, 13, 14, 15, 16,17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100,100-500, and 500-1,000, for example.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” or “contain”) are inclusive or open-ended and do not exclude additional, unrecited elements or method steps.

The term “or combinations thereof” as used herein refers to an permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, SAC, or CAB. Continuing with this example, expressly included are combinations that contain repeats of one or more item or term, such as BB, AAA, AAB, BBC, AAABCCCC, CBBAAA, CABABB, and so forth. The skilled attisan with understanding that typically there is 1).-0 Um.it 011. the number of items. t.lt tennS. If any combination, unless otherwise apparent from the context.

Throughout this application the term “about” is used to indicate that a value includes the inherent variation of error for the composition, the method used to administer the composition, or the variation that exists among the study subject. As used herein the qualifiers “about” or “approximately” are indicated to include not only the exact value, amount, degree, orientation, or other qualified characteristic or value, but are intended to include some slight variations due to measuring error, manufacturing tolerances, stress excelled on various parts or components, observer error, wear and tear, and combinations thereof, for example. The term “about” or “approximately”—where used herein when referring to a measurable value such as an amount, a temporal duration and the like, is meant to encompass, for example, variations of J101h, or +˜5%, or ±1%, or ˜1::0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event 01 circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiments.

The term “pharmaceutically acceptable” refers to compounds and compositions which are suitable for administration to humans and/or animals without undue adverse side effects such as toxicity, irritation and/or allergic response comrnensmate with a reasonable benefit/risk ratio. The compounds of the present disclosure may be combined with one or more pharmaceutical acceptable excipients—including :a:n:iers, vehicles, and dialects which may improve solubility, deliverability, dispersion, stability, and/or conformational integrity of the “compounds or conjugates thereof”. As used herein a “pharmaceutically acceptable carrier” is a pharmaceutically acceptable solvent, specialiazing agent:, vehicle, or diluent for delivering the active agents of the present disclosure to a subject. The carrier may be liquid or solid and is selected with the planned manner of administration in mind. Examples of pharmaceutically acceptable carriers that may be utilized with the active agents disclosed herein include but are not limited to polyethylene glyceryl (PEG) of various molecular weights, liposomes, ethanol, dimethyl sulfoxide (DMSO), aqueous buffers, oils, and combinations thereof.

As used herein, “pure,” or “substantially pure” means an object species; is the predominant species present (i.e., on a molar basis it is more abundant than any other object species in the composition thereof, and particularly a substantially purified fraction is a composition wherein the object species comprises at least about 50 percent (on a molar basis) of an macromolecular species present. Generally, a substantially pure composition will comprise more than about 80° A, of all macromolecular species present in the composition, more particularly more than about 85%, more than about 90%, more than about 95%, or more than about 99%. The term “pure”or “substantially pure” also refers to preparations where the object species is at least 60% (w/w) pure, or at least 70% (w/w) pure, or at least 75% (w/w) pure, or at least 80% (w/w) pure, or at least 85% (w,w) pure, or at least 90% (w/w) pure, or at least 92% (w/w) pure, or at least 95% (w/w) pure, or at least 96% (w/1,v) pure, or at least 97% (w/w) pure, or at least 98% (w/w) pure, or at least 99% (w,w) pure, or 100% (w/w) pure.

Non-limiting examples of animals within the scope and meaning of this term include dogs, cats, rats, mice, guinea pigs, chinchmas, horses, goats, cattle, sheep, zoo animals, Old and New World monkeys, non-human primates, and humans.

“Treatment” refers to therapeutic treatments. “Prevention” refers to prophylactic or preventative treatment measures or reducing the onset of a condition or disease. The term “treating” refers to administering the composition to a subject therapeutic purposes and/or for prevention. Non-limiting examples of modes of administration, include oral, topical, retrobulbar, subconjunctival, nonsdemal, parenteral, subcutaneous, intranasal, intramuscular, intraperitoneal, intravitreal, and intravenous routes, including both local and systemic applications. In addition the compositions of the present disclosure may be designed to provide delayed, controlled, extended, and/or sustained release using formulation techniques which are well known in the art.

The term atopical, is used herein to define a mode of administration through an epitfa:lial surface, such as but not limited to a material that is administered by being applied externally or internally to a surface.

The terms “therapeutic composition ” and “pharmaceutical composition” refer to an active agent-containing composition that may be administered to a subject by any method known in the art or otherwise contemplated herein. Wherein administration of the composition brings about a therapeutic effect as described elsewhere herein. In addition, the compositions of the present disclosure may be designed to provide delayed, controlled, extended, and/or sustained release using formulation techniques which are well known in the art.

The term “effective amount” refers to the amount of an active agent of the present disclosure which is sufficient to exhibit a detectable therapeutic or treatment effort in a subject without excessive adverse side effects (such as substantial toxicity, irritation and allergic response) commensurate with a reasonable belle fitk isk ratio when used in the manner of the present disclose. The effective amount for a subject will depend upon the subject's type, size and health, the nahlre and severity of the condition to be treated, the method of administration, the duration. Of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

The term “ameliorate” means a detectable or measurable improvement in a subject's condition or symptom thereof. A detectable or measurable improvement includes a subjective or objective decrease, nthlction, inhibition, suppression, limit, or control in the occurrence. frequency, severity, progression, or duration of the condition, or an improvement in a symptom or an underlying cause or a consequence of the condition, or a reversal of the condition. A successful treatment outcome can lead to a “therapeutic effect,” or “benefit” of ameliorating, d-e.,1eitsiQg, reducing inhibiting, suppressin limiting, controlling or preventing the occurrence, frequency, severity, progression, or duration of a condition, or consequences of the conditioning a subject.

A decrease or reduction in worsening, such as stabilizing the condition is also a successful treatment outcome. A therapeutic benefit therefore need not be complete ablation or reversal of the condition, or any one, most or all advenw symptoms, implications, consequences or underlying causes associated with the condition. Time, a satisfactory endpoint may be achieved when there is an incremental improvement such as a partial decrease reduction, inhibition, suppression, ihxit., eotrtn)_{1 1}″ ii, irrelevant to the occurrence, frequency, severity, progression, or duration, or inhibition or reversal of the condition (e.g., stabilizing), over a short or long duration of time (e.g., seconds, minutes, hours).

In at least certain non-limiting embodiments, the present disdmmre includes compositions and methods of treating a subject for arterial stiffness and/or hypertension, and/or aging effects, with an N-phenyl-1 H-indole-3-carboxamide derivative. In certain embodiments the N-phenyl-1 H-indole-3-carboxamide derivative is at least one N-halophenyl-1H-indole-3-carboxamide selected from the group: N-chlorophenyl-1H-indole-3-carboxamide, N-fluorophenyl-1H-indole-3-carboxamide, N-bromopiten-yl-1H-indole-3-carboxamide, and N-iodophenyl-1H-indole-3-carboxamide, or pharmaceutically-acceptable salts thereof, wherein the halogen is on the 2, 3, 4, 5, or 6-carbon position on the phenyl ring.

In certain embodiments, the N-halophenyl-1H-indole-3-carboxamide active agent is an N-(2-halophenyl)-1H-indole-3-carboxamide, or a pharmaceutically-acceptable salt thereof having Formula 1;

formula I
wherein R is, selected from the group consisting of chlorine (Cl), fluorine (F), bromine (Br) and iodine (I).

In certain embodiments, the N-halophenyl-1H-indole-3-carboxamide active agent, or pharmaceutically-acceptable salt thereof, may have a halogen on two or more of the carbons of the phenyl ring, such as on two, three, four, or five carbons selected from the 2, 3, 4, 5, and 6-carbon positions of the phenyl ring, wherein the two or more halogens may be selected from Cl, F, Br, and I, and combinations thereof. The two or more halogens may be the same (e.g., both Cl) or different (e.g., Cl and F).

In at least one embodiment, the present disclosure is directed to a method of treating hypertension and/or arterial aging in a subject in need of such therapy, comprising: administering to the subject an effective amount of a composition comprising an N-halophenyl-1 H-indole-3-carboxamide or a pharmaceutically-acceptable salt thereof, wherein the N-halophenyl-1H-indole3-carboxamide has ahalogeu on at least one of the 2, 3, 4, 5, and 6-carbon positions of the phenyl ring, wherein the halogen is selected from the group consisting of chlorine (Cl), fluorine (F), bromine (Br), and iodine cp. The N-balophenyl-1H-indole-3-carboxamide or pharmaceutically-acceptable salt thereof may be represented by Formula I above, wherein R in Formula I is selected from the group consisting of Cl, F, Br, and L. For example, R may be CL.

In at least one embodiment, the present disclosure is directed to a method of treating arterial aging in a subject in need of such therapy. Comprising administering to the subject an effective amount of a compound that increases arterial elastin production, the compound comprising an N-halophenyl-1H-indole-3-carboxamide, or a pharmaceutically-acceptable salt thereof, wherein the N-halophenyl-1H-indole-3-carboxamide has a halogen on at least one of the 2, 3, 4, 5, and 6-carbon positions of the phenyl ring; wherein the halogen is selected from the group consisting of chlorine (Cl), fluorine (F), bromine (Br), and iodine (I). The N-balophenyl-1H-indole-3-carboxamide or pharmaceutically-acceptable salt thereof may be represented by Formula I above, wherein R in Formula I is selected from the group consisting of Cl, F, Br, and L. For example, R may be Cl. The compound may further decrease arterial collagen production.

As noted above, the present disclosure describes methods of administering compositions comprising active agents for the treatment of certain diseases and conditions, including but not limited to, arterial stiffness, hypertension, diabetes, and anti-aging effects, in a subject without wishing to be bound by them, it is believed that the active agents alter the physiological process of these conditions and diseases by activating DNA demethylases and increasing expression of Klotho protein. Further, the active agents and methods of use thereof are also directed to methods of providing anti-aging treatments. Without wishing to be bound by theory, it is believed that the active agents cause arterial remodeling by decreasing collagen production, and increasing elastin production in the arterial tissues of subject. The methods disclosed herein are therefore directed to (1) the methods of treating and attempting arterial stiffening and/or hypertension by altering the pathological process of arterial stiffening and hypertension, and (2) to methods of providing arterial remodeling by decreasing collagen production and increasing elastin production letfid tissues thereby res11W. ig i:ri autf,:aging effects in the subject. The active agents described herein may be used to treat any disease or condition characterized by arterial stiffening, and/or by loss of elastin, including but not limited to, hypertension, aortic disease, cardiovascular disease, including heart failure (such as, congestive heart failure), diabetes, kidney disease, osteoporosis, Alzheimer's disease, infertility, and emphysema.

In certain embodiments, the present disclosure is directed to compositions and conjugates comprising the active agent, wherein the compositions and conjugates may be utilized in methods of treating arterial stiffening and hypertension and for providing anti-aging treatments for providing arterial remodeling, and for treating other diseases and conditions identified herein. The inventive concepts disclosed herein also kits that include said active agent and compositions and conjugates thereof.

The present disclosure is also directed to methods of administering an effective amount of active agent to a subject, wherein the effective amount of active agent is sufficient to cause activation of DNA demethylases and attenuation of aging-related arterial stiffening and hypertension, and/or decreasing collagen production and increasing elastin production in arterial tissues for anti-aging treatments for providing arterial remodeling.

Without wishing to be bornid by theory it is believed that the active agent increases DNA demethylase activity and decreases methylation of the Klotho gene, thereby increasing expression of Klotho protein. The resulting increase in circulating levels of Klotho protein cm.i.sed by administration of the active agent is thought to reduce accumulation of stiffer collagen and reduce degeneration of compliant elastin fibers. Further, the ine.n. Klotho expression attenuates the aging increased activity and expression of rvi:MP2 (arterial matrix metalloproteinase-2), MM P9 (arterial matrix metalloproteinast, . . . , 9) and the aging increased expression of TGF-Pt (transforming growth factor Pl), TGF-3 (transforming growth factor--3t RUNX2 (nmr, related. transcription factor 2), and ALP (alkaline phosphatase) to inhibit arterial fibrosis and stiffness.

In certain embodiments, the methods of the present disclosure are directed to treating arterial stiffness and/or hypertension in a subject, such as a human, or other mammal, which is in mlel of such therapy, by administering a the pentkally-effective amount of an active agent, as disclosed herein. Without wishing to be bound by theory it is believed that the active agent has its effect by increasing the activity of the klotho gene thereby causing an increase in the expression of Kiotho protein which leads to a reduction in arterial collagen ptpdtwtkm miri tli.) foctease hiarterial elastin production, thereby, attenuating arterial stiffness and hypertension,

In certain embodiments, the methods of the present disclosure are directed to causing arterial remodeling (anti-aging) in a subject, such as a human or other manual, which is in need of such therapy by administering a therapeutically—effective account of the active agent, as disclosed herein. Without wishing to be bound by theory it is believed that the active agent has its effect by increasing the activity of the klotho gene thereby causing an increase in the expression of Klotho protein which leads to a reduction in arterial collagen production and an increase in arterial elastin production, thereby causing arterial remodeling, and the attenuation of arterial stiffness and hypertension.

Moreover, as noted, administration of the active agents of the present: disclosure cause an increase in production Klotho protein Klotho protein is known as a treatment for diabetes or diabetes-related diseases or conditions (e.g., see. Published PCT application WO 2014/152993 A1, expressly incorporated herein by reference in its entirety in cellular embodiments therefore, the presently disclosed active agents; by virtue of their effect in causing enhanced production of Klotho protein, but be used to treat a subject having diabetes or a diabetes related disease or condition—such as but not limited to Type 1 diabetes mellitus (T1DM), Type 2 diabetes mellitus (T2DM), and hyper-insulin (pre-diabetes). Diabetes related diseases or conditions include, but are not limited to: obesity; peripheral arterial disease (PAD)) of the anus, legs, and foot—foot ulcers—hypertension; diabetic neuropathy—diabetic retinopathy; diabetic kidney disease; ketoacidosis; and hyperosmolar hyperglycemia nonketotic syndrome (HHNS).

T2DM generally progresses through several phases from pre-diabetes to full-blown insulin dependence. As elevated glucose levels occur in the body, it will place a higher demand on insulin secreting pancreatic beta cell to produce insulin and restore glucose homeostasis and alleviate postprandial spikes). This increased demand to take insulin leads to endoplasmic reticulum stress (ERS) in the beta cells. ERS can also result in phosphorylation of the insulin receptor, which attenuates insulin efficacy, causing insulin resistance and, as a result further increases the demand for insulin production. As this ERS in the beta cells continues, it will eventually lead to a loss in beta cell mass, which has been observed in autopsies of T2DM individuals. Since beta cells do not appear to be regenerated in the pancreas, this loss in beta cell mass leads to the folly insulin dependent phenotype of later stage T2DM patients,

Preservation of pancreatic beta cells thus maintains beta cell mass due to amelioration of ERS. The latter serves to prevent one of the most deleterious outcomes in T2DM, which is loss of beta cell mass in regard to T1DM, amelioration of beta cell loss deleting the early stages of the disease helps preserve beta cell mass while treatments against beta cell-killing autoimmunity T1DM are mounted. Therefor, in at least one embodiment, the present disclosure includes a method of preserving beta cell mass in a subject suffering from diabetes or a diabetic related condition by administering to the subject at least one of the active agents described herein. The preservation of beta cell mass in the subject can be shown by an increase or stabilization in the amount of insulin production and/or C-peptide production in the subject: (as measured by blood or urine tests), in one suitable assay beta cell mass is indirectly calculated by determining the ratio of C-peptide to glucose following oral glucose ingestion particularly as measured 1.5 minutes after glucose ingestion (Meier et al., Diabetes 2009; 58, 1595-1603). Alternatively, beta cell mass preservation can be indirectly calculated by using the Homeostasis model assessment (HOMA) index (Matthews et al., Diabetologia 1985; 28: 412-419). Additionally, in at least one embodiment, the administration of an active agent disclosed herein results in a blood hemoglobin A1C which is less than about: 7% in the subject.

The active agents of the present disclosure may be administered to a subject by any method known in the art, indicating but not limited to, oral, topical, transdermal, parenteral, subcutaneous, intranasal, intramuscular, intraperitoneal, and intravenous routes, and including both local and systemic applications. The active agents may also include a pharmaceutically acceptable carrier, such as a solvent, suspending agent, or vehicle for delivering the compositions or conjugates to the subject. In addition, the active agents disclosed herein may be configured to provide delayed or condemned release using simulation techniques which are well known in the art.

The present disclosure is also directed to a pharmaceutical composition comprising an effective amount of an active agent in combination with a pharmaceutically acceptable carrier. As used herein the term “effective amount” refers to an amount of a biologically active molecule or conjugate or derivative thereof sufficient to exhibit a detectable therapeutic effect without undue side effects (such as toxicity, irritation and allergic response) commensurate with a reasonable benefit/risk evaluation when used in the matter of the present disclosure. As noted above, the therapeutic effect may include, for example attenuating arterial stiffening and hypertension, and rebuilding arterial remodeling. As one of ordinary skill in the art individually appreciate the effective amount for a particular subject, such as an adult male, adult female, or child, will depend upon the type of subject, the subject's size and health, the nature and severity of the condition to be treated the method of administration, the duration of treatment, the nature of any additional therapy or treatment employed, the specific formulations employed, and the like.

The term “gene” is used herein for simplicity to refer to a functional protein, polypeptide, or peptide encoding DNA unit. As will be understood by those in the and this functional term includes genomic sequences, cDNA sequences or combinations thereof. “isolated substantially away from other ending sequences” means the gene of interest forms the significant part of the coting region of the DNA segment, and that the DNA segment does not contain other notHeleant large portions of naturally-occurring coding DNA, and as large chromosomal fragments or other functional genes or DNA coding regions. This refers to the DNA segment as originally isolated, and does not exclude genes or coding regions later added to, or intentionally left in the segment by a person.

The term “polypeptide” means a molecule comprising a series of amino acids linked through amide linkages along the alpha carbon backbone. Modifications of the peptide side chains may be present, along with glycosylation, hydroxylation and delhi. Additionally, other non-peptide molecules, including lipids and small molecule agents may be attached to the polypeptide.

The nucleic add segments of the present disclosure, regardless of the length of the coding sequence itself may be combined with other DNA sequences, such as promoters, polyadenylation signals, additional restriction enzyme sites, multiple cloning sites, epitope tags, polyhistidine regions, other coding segments and the like. Such that the overall length may vary considerably. It is, therefore, contemplated that nucleic acid fragments of almost any length may be employed with the total length preferably being limited by the ease of preparation and use in the intended recombinant DNA protocol.

Practice of the methods of the presplit, disclosure may comprise administering to a subject an effective amount of the active agent in any suitable systemic and for local formulation in an amount effective to deliver the dosages listed herein, or other acceptable dosages as determined by the attending physician. An effective amount of an active agent of the present disclosure will generally contain sufficient active agent to deliver in current embodiments, from about 0.1 pg/kg to about 100 mg/kg (mass of active agent/body weight of the subject). Particularly, the composition will deliver about 0.5 pg/kg to about 50 mg/kg, and more particularly about 1 μg/kg to about 10 mg/kg. The dosage can be administered, for example but not by way of limitation, on a onetime basis, or administered at multiple times (for example but not by way of limitation, from one to five times per day, or once or twice per week), or continually via, a venous drip, depending on the desired therapeutic effect in one non-limiting embodiment of a therapeutic method, the active agent is provided in an IV infusion in the range of from about 0.1 pg/kg to about 10 mg/kg of body weight once a day.

Administration of the active agent used in the pharmaceutical composition or to practice the method of the present disclosure can be carried out in a variety of conventional ways, such as, but not limited to, orally, by inhalation, rectally, or by cutaneous, subcutaneous, intraperitoneal, or intravenous injection. Oral formulations may be formatted such that the active agent passes through a portion of the digestive system before being released, for example it may not be released until reaching the small intestine or the colon.

When an effective amount of the active age 11 (is administered orally, it may be in the form of a solid or liquid preparations such as capsules, pills, tablets, lozenges, melts, powders, suspensions, solutions, or emulsions. Solid unit dosage forms can be capsules of the ordinary gelatin type containing, for example, surfactants, lubricants, and inert fillers such as ladose, sucrose, and comsuirch, or the dosage forms can be sustained release preparations. The pharmaceutical composition may contain a solid carrier, such as a gelatin or an adjuvant. The tablet, pseudo and powder may contain from about 0.05 to about 95% of the active substance compound by dry weigh, thus administered in liquid form a liquid carrier such as vapor, petroleum, oils of anfrilal or plant origins such as peanut oil, mineral oil, sesame oil, or synthetic oils may be added. The liquid form of the pharmaceutical composition may further contain physiological saline solution, dextrose or other saccharide solution, or glycols such as ethylene glycol, propylene glycol, or polyethylene glycol. When administered in liquid form, the pharmaceutical composition particularly contains from about 0.005 to about 95% by weight of the active tigent. For example, u dose of about 10 mg to about 1000 mg once or twice a day could be administered orally.

In another embodiment, the active agents described herein can be tableted with conventional tablet bases such as lactose, sucrose, and maltose combination with binders such as acacia, eon starch or gelatin, disintegrating agents such as potato starch or alginic acid and a lubricant such as stearic acid or magnesium stearate liquid preparations are prepared by dissolving the active agent in an aqueous or non-aqueous pharmaceutically acceptable solvent which may also contain suspending agents, sweetening agents, flavoring agents, and preservative agents as are known in the art.

For parenteral administration, for example, the active agent of the present disclosure may be disposed in a physiologically acceptable pharmaceutical carrier and administered as either solution or a suspension. Illustrative of suitable pharmaceutical carriers are water, saline, dextrose solutions, fructose solutions, ethanol, or oils of animal, vegetative, or synthetic origin. The pharmaceutical carrier may also contain preservatives and buffers as are known in the art.

When an effective amount of the active agent is administered by intravenous cutaneous, or subcutaneous injection, the active agent may be in the form of a pyrogen-free, parenterally acceptable are oils solution or suspension. The preparation of such parenterally acceptable solutions living due and to pH isotonicity, stability, and the like, is well within the skill in the art. A particular pharmaceutical composition for intravenous, cutaneous, or subcutaneous injection may contain, in addition to the active agent, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present disclosure may also contain stabilizers, preservatives, butters, antioxidants, or other additives known to those of skill in the art.

As noted, particular answers and modes of administration can be determined by one skilled in the art. One skilled in the art of preparing translations can readily select the proper form and mode of administration, depending upon the particular characteristics of the active agent selected the condition to be treated, the stage of the condition, and other relevant circumstances using formation technology known in the art described, for example, in Remington II Science and Practice of Pharmacy 2 pt. ed.

Additional pharmaceutical methods may be employed to control the duration of action of the active agent. Increased half-life anchor controlled release preparations may be achieved through the use of polymers to conjugate complex with and/or absorb the active agent described herein. The controlled delivery and/or increased half may be achieved by selecting appropriate macromolecules (for example but not by way of limitation, polysaccharides, polyesters, polyamino acids, homopolymers polyvinyl pyrrolidone, ethylenevinylacetate, methylcellulose, or carboxymethylcellulose, and acrylamides such as N-(2-hydroxypropyl) methacrylamide), and the appropriate concentration of macromolecules as well as the methods of incorporation, in order to control release. The active agent may also be ionically or covalently conjugated to the macromolecules described above.

Possible methods useful in controlling the duration of action of the active agent by controlled release preparations and half-life is incorporation of the active agent into particles of a polymeric material such as polyesters, polyamides, polyamine adds, hydrogels, poly(lactic acid), ethylene vinyl acetate copolymers, copolymers micelles of for example, polyethylene glycol (PEG) and poly(l-aspamunide).

It is also possible to entrap the active agent in microcapsules prepared, for example, by conservation techniques or by interfacial polymerization (for example, hydroxymethylcellulose or gelatin-microcapsules and poly-(methylmethacyfate) micro capsules; respectively), in colloidal drug delivery systems (for example, Liposomes, albumin microspheres, microemulsions, nanoparticles, and microcapsules), or in macroemulsions. Such techniques are well known to persons having ordinary skill in the art.

When the active agent is to be used as an injectable material, it can be formulated into a conventional injectable carrier. Suitable carriers include biocompatible and pharmaceutically acceptable phosphate buffered saline solutions, which are particularly isotonic.

The active agent may be formulated in a composition that induces a sterile diluent, *Nbich may contain materials generally recognized for approximating physiological conditions and/or as required by governmental regulation. In the respect, the sterilization may contain a buffering agent to obtain a physiologically acceptable pH such as sodium chloride, saline, phosphate-buffered saline, and/or other substances which are physiologically acceptable and/or same for use in general, the material for intravenous injection in humans should conform to regulations established by the Food and Drug Administration, which are available to those in the field. The pharmaceutical composition may also be in the form of an aqueous solution containing many of the same substances as described above.

The active agents of the present disclosure can also be administered as a pharmaceutically acceptable acid additional, followed by reaction with inorganic acids such as Hydrochloric acid; hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with inorganic base such as sodium hydroxide, Ammonium hydroxide, potassium hydroxide, and organic bases such as mono-, di-, tri-alkyl and aryl amines, and substituted ethanolamines.

Several embodiments of the present disclosure, having now been a generally described, will be more readily understood by reference to the following example, which is included merely for purposes of illustration of certain aspects and embodiments of the present disclosure and is not intended to be limiting. Those skilled in the art will promptly recognize suitable and appropriate variations from the various compositions, compounds, components, procedures and methods described in the example.

### EXAMPLE

The object of this example is to described the effect of one embodiment of an active agent of the present disclosure on arterial stiffness and hypertension in a subject such as aged (old) mice for example. In this example the active agent is N-(2-chlorophenyl)-Ul-indde-3-carboxamide, but it is to be understood that the methods of the present disclosure are not to be limited to this particular compound. Other N-phenyl-1H-indole-3-carboxamide derivatives, including but not limited to other N-halophenyl-1H-indole-3-caboxamides can effect the same results. This example demonstrates that aging-related arterial stiffening and hypertension are partially attributed to increased DNA methylation. The active agent is shown to increase Klotho expression and attenuate arterial stiffening and hypertension in old mice. Thus, the active agent is an effective raps agent for aging-related arterial stiffening and hypertension. All older use of the active agent is to cause a decrease in arterial collagen production and an increase in aerial elastin production thereby providing arterial remodeling which serves as an anti-aging treatment.

In this example, old (“aged”) mice (24-30 months of age), were treated with or without Compom1<1H 0.5 mg/kg, (P daily) for 2 weeks while adult mice (12 months of age) without treatments were used as controls. Compound H in this non-Limiting example is N-(2-′- fluorophenyl)-1H-indole-3-arboxamide.

Pulse wave velocity (PWV), n_direct weasm-e Qf arterial stiffness, and blood pressure (BJ>) were increased significantly in aged mice. Notably, the active agent reversed the age-related increases in PWV and BP within 2 weeks of treatment. The active agent effectively increased secreted Klotho levels in both kidney and seen through increasing ONA demethylase activity to lysinemethylation of Klotho gene. Aging-related arterial stiffness was associated accumulation of stiffer collagen and degeneration of compliant elastin fibers. In addition, the activity and expression of IV1MP2, MMP9, and the expression of TGF-1 TGf-P3, RUNX2 and ALP were increased in aortas of aged mice. These changes were attempted by the active agent. Mechanistically, the silent information, regulator T1 enzyme-AMP-activated protein kinase endothelial nitric oxide synthase (SIRT1-AMPK-eNOS) pathway may also take part in the therapeutic effort of the active agent.

The active agent effectively attenuated the increases in PWV and BP in old mice, indicating that it is an effective therapeutic agent for arterial stiffness and hypertension. The active agent also electively decreased collagen production and increased elastin production (as shown in FIG. 4B), indicating that it is effective in arterial remodeling for use as an anti-aging treatment.

### Method

Animal study protocols. This example was performed according to the guide lines of the National Institute of Health on the care and use of laboratory animals and approved by the institutional Animal Care and Use Committee of University of Oklahoma Health Science Center. AU mice were housed in cages at room temperature (2S±PC) and were provided with Purina laboratory chow No 5001) and tap Water and lithium. 6 adult mice (12 months) and 20 old mice (24-26 months) mice were next in this study. The old (aged) mice were randomly divided into three subgroups and each group had 6 or 7 mice. One subgroup received compound H (10 mg/kg/day1, IP, Enamine LLC, Monmouth Jct., NJ) and one group received an equal volume of DMSO (dimethyl sulfoxide, 5%) and served as a control. The third group received no treatment Blood pressure was measured before and after treatment with the active agent at 1 week and 2 week. Pulse wave velocity (PWV) was measured after 2 weeks treatment with active agent all animals were sacrificed and perfused transcardially with PBS under deep anesthesia (ketamine xylazine, 90/10 mg, IP). The aortas were then quickly removed, washed and cut into pieces for subsequent analyses.

Cell culture and treatment: MOVAS (ATCC®CRL-2797) is a continuous mouse aortic vascular smooth muscle cell type that has been demonstrated to reach VSMC-like phenotype, including a spindle cell methodology and the expression of VSMC-specific markers such as smooth muscle reaction and S:M22-a, MOVAS were cultured in Dulbecco's modified Eagle's medium (DMEM:) supplemented with 10% heat-inactivated fetal bovine serum (FBS) and 2 mM L-glutamine. After cells confluent, the media were switched to DMEM with or without 2° A1 serum, and the cells were then treated with 10 μM active agent or 5 nM secreted Klotho for another 16 h then harvested for western blot analysis.

Measurement of Pulse Wave Velocity: Aortic PWV was measured as described previously. Briefly, mice were anesthetized under 2% isoflurane in a closed circuit anesthesia machine (SonmoSuite, Kent Scientific, Torrington, CT) for −1-3 nun. Anesthesia was maintained via nose cone, and mice were secured in a supine position on a heating board (˜37° C) to maintain body temperature. Velocities were measured with 6-mm crystal 20-100 Hz Doppler probes (Indus Instruments, Webster, TX) at the transverse aortic arch and 3.5 cm distal in the abdominal aorta and collected using Doppler signal processing workstation (Indus instruments). Absolute pulse arrival times were indicated by the sharp upstroke or foot, of each velocity waveform. Aortic PWV is then calculated as the quotient of the separation distance, assessed to the nearest half millimeter by engineering caliper and difference in absolute arrival times.

Measurement of Blood Pressure: Blood pressure was measured by the volume-pressure recording (VPR) tail-cuff method with slight (28° C.) but not heating of the tail using it CODA none-invasive blood pressure monitoring system (Kent Scientific). This method of measurement has been validated by using a telemetry system. Animals were gently handled and trained for the VPR tail-cuff measurement to minimize handling stress. No signs of stress were observed during BP measurements. The operator was also strictly trained for the measurement procedure. At least 20 stable cycles data were obtained for the result analysis for each measurement.

Histological and Immunohistochemical Staining: Thoracic aortas were quickly excised and placed in cold (4′T) physiological saline solution. Three Trimester rings with perivascular tissue intact were removed from the thoracic aorta directly distal to the greater curvature of the aortic arch. Aorta rings were post-fixed in 4% paraformaldehyde, embedded in paraffin and sectioned at 5 cm thickness, Collagen was quantified by Masson' s trichrome staining as described previously. The black staining represented collagen deposition. A series of 5-10 sections of each mouse (5 mice per group) were examined and photographed using an Olympus microscope coupled with a digital color camera. Blue-stained collagen areas were quantified with ImageJ (NIH, Bethesda, USA) film 4-5 regions per section. The same threshold was used for each photo to make sure they are comparable. Elastin was assessed by Immunohistochemical visualization. Briefly, sections are washed and incubated in primary antibody against elastin (1:50, Abeam, Cambridge, MA, USA) or negative control (2.5% horse serum, Vector Labs) overnight and elastin was visualized using the appropriate secondary antibody. Finally, series of 5˜10 sections were examined and photographed using an Olympus BH-L, microscope coupled with a digital color camera. Elastin levels were quantified with ImageJ (NIH, Bethesda, MA) from 4-5 regions per section. The same threshold was used for each photo to make sure they are comparable.

Western Blot Analysis: Protein samples from the thoracic aorta were prepared in Lysis buffer as described previously. The proteins (40-50 mg were resolved by SOS-PAGE and transferred to a nitrocellulose membrane (Bio-Rad). The membrane was then incubated overnight (4° C.) with a primary antibody against collagen-1-elastin. M1′v1P2; MMP9, ALP (Abeam1 1:1000), TGF-1, TGF˜N3 (Santa Cmz, 1:100). RUX2, or aNTulmliu (Cell Signaling. 1:1000). Goat autimouse or goat anti rabbit horseradish peroxidase (1:2000 . . . 1:5,000; Santa Cruz Biotechnology) was used as a secondary antibody find incubated for 1 hour at room temperature. Specific proteins were detected by chemiluminescent methods using Amersham™ western blotting detection reagents (GE Healthcare, UK). Protein abundance on western blots was quantified densitometry using Image lab software (Bio-Rad, Hercules, CA).

Reverse transcription-PCR (RT-PCR): Total RNA was extracted using a Direct˜zol T!vl RNA Miniprep kit (Zymo Research, Irvine CA) from kidney of adult and aged mice. The first smtrrd qDNA was synthesized from 500 ng total RNA by using an iScript cDNA synthesis kit (Bio-Rad). The gene-specific primers for Klotho are shown in Table 1 in the parent application, U.S. Provisional Application No. 62/509,818.

The PCR conditions for the Klotho and (ht\ctiu primers were as follows: bold the 0.5 min at 94° C. followed by 30 cycles consisting of denaturation at 94° C (30 s), appealing at 57° C. (30 s). and elongation at 72° C. (1 min). The amplified products were subjected to electrophores is on a 1% agarose gel. Each reaction was performed in triplicates. The gene expression was calculated as band intensity of Klotho band intensity of action and plotted after localization to the control group.

Methylation Analyses of Klotho Gene: The methylation status of CpG islands of mice Klotho gene was analyzed using methylation-specific PCR (MSP) and bisulfite sequencing. First, the genomic DNA was extracted kidney of adult and aged mice with tissue DNA Kit (Omega Norcross, GA.) and modified by bisulfite treatment (EZ DNA Methylation (fold Kit, Zymo Research) for MSP analyses with gene promoter-specific primer pair that recognize the methylated and unmethylated CpG sites. The PCR product of genomic DNA without bisulfate treatment, with primers located in the promoter region of the mouse Klotho gene, was used as the inputted control for the MSP. The PCR producers were visualized by ethidium bromide staining in 2% agarose gels, and the dosimetry intensity corresponding to each baud was quantified. Each reaction was performed in triplicates. The methylation index was calculated as (band intensity of MSP with methylated primers)/(band intensity of inputted control). The methylation index was plotted after normalization to the control group. The sequences of MSP primers and the amplification program are summarized in Table 2 in the parent application, U.S. Provisional Application No. 62/509,818. The PCR program conditions for the MSP primers are shown in Table below.

### Table

Measurement of DNA Demethylase and DNA Methylation force Activity. The DNA demethylase and DNA methyltransferase activity was measured by using DNA demethylase activity qualification kit (Abeam˜Cambridge, MA) and methyltransferase coloritnetxic assay kit (Cayman Chemical Company, Ann Arbor, Michigan). Protein extracts from the thoracic aorta were prepare in lysis buffer. Assay procedures were followed by the manufacturer's protocols. The absorbance was read at 450 um for DNA methylene activity assay and 510 nm for DNA methyltransferase activity using BioTeck Multi-Mole Microplate Readers.

Measurements for MMP2 and MMP9 Activity. MMP2 and MMP9 activity were measured by Zymogram PAGE (Bio˜-Rad). Briefly Lysates from thoracic aorta were quantified using BCA assay (Pierce) and prepared under non-reducing, non-denaturing conditions. Protein from lysates was separated on a zymognun gel containing Gelatin (Bio-Rad). After warming, the gd was incubated in the Zymogram Renaturing Buffer with gentle agitation for 30 minutes at room temperature. Next the gel was equilibrated in 1× developing for 30 minutes on a shaker at room temperature. Then, fresh developing buffer was replaced and the gel was incubated at 37° C. overnight to develop. The next day, the gel was washed 3×, 5 minutes each, in doubly distilled water, then stained with brilliant blue R₂₅₀ for 30 minutes. Gels are de-stained with an appropriate Co-omassie R-250 destaining solution (Methanol: Acetic acid: Water (50: 10:40). Areas of protease activity appear as clear bands against a dark blue background where the protease has digested the substance.

Statistical Analysis: Initiative data is presented as the Means±SE. Differences between experimental groups were examined by one way analysis of variance (A NOVA) followed by the Bonferroni posHest using Prism software (GraphPad). For aH analysis, p<0.05 were considered statistically significant.

### Results

Activation of demethylase by the active agent attenuated arterial stiffness and hypertension in aged mice. Arterial pulse wave velocity (PWV) is a direct measure of arterial stiffness. The widening pulse pressure (the numeric difference between systolic and diastolic blood pressure) seen with aging is another direct indicator of arterial stiffness. PWV of aged mice was increased significantly compared to that of adult mice (3.11±0.15 mis vs. 2.27±0.13 mis, p<0.01) (FIG. 1A). Pulse pressure of aged mice was also significantly increased compared to that of adult mice in deferent time point (FIG. 1B), indicating that aging causes arterial stiffness. However increasing circulating levels of Klotho by the active agent decreased P1/V and pulse pressure after 2 week treatment in aged mice FIGS. 1A and B). Besides reducing arterial stiffness, the active agents decreased system blood pressure (FIG. 1C), diastolic blood pressure (FIG. 1D), and niean blood pressure (FIG. 1E) in aged mice to the added levels after 2 weeks treatment. Therefore, increasing circulating levels of Klotho by the active agent reduced arterial stiffness and Blood pressure in aged mice.

The active agent decreased the DNA, hypermethylation of Klotho gene in aged mice. DNA demethylase activity and DNA methyltransferase activity was then measured. The DNA demethylase activity was significantly decreased and the DNA methyltransferase activity was significantly increased in aged mice (24-26 months old) compared to that of adult mice (12-16 months old) FIG. 2A and B). The active agent increased the DNA demethylase activity which was decreased in aged mice, while the active agent did not affect the DNA methyltransferase activity (FIG. 2A and B). The methylation of the Klotho gene was then measured. The methylation of Klotho gene was significantly increased in aged mice compared to that of adult mice (FIG. 2C). Than, the active agent inhibited the methylation of Klotho gene which was decreased in aged mice (FIG. 2C).

The active agent increased expression of secreted Klotho in aged mice. Secreted Klotho, acting as a hormone, shows different frictional from full-length Klotho. The active agent significantly increased secreted Klotho protein expression in kidney of 24-26 month-old mice, while the active agent did not affect full-length Klotho expression (FIG. 3A). The circulating levels of secreted Klotho were also markedly increased by the active agent (FIG. 3B). Besides protein expression, the active agent also increased secreted Klotho mRNA expression in kidney (FIG. 3C). Taken together, the active agent increased secreted Klotho mRNA and protein expression in aged mice.

Further, increasing circulating levels the Klotho by the active agent prevented accumulation of stiffer collagen and degeneration of collagen elastic fibers in aortas of aged mice. The arterial collagen ruled elastin levels were measured by immunostaining and western blot assays. The immunostaining assay showed that aortic collagen levels were increased significantly ill aged mice (FIG. 4A). Aging induced collagen deposition was mainly found in the medial and adventitial layer of the aorta. Aortic elastin levels were decreased significantly in aged mice (FIG. 4A). Western blot analysis confirmed that aging upregulated collagen I expression but downregulated elastin levels in aortas (FIG. 4B). The ratio of elastin to collagen in aortas was markedly decreased in aged mice (FIGS. 4A-4B), indicating the aging causes arterial remodeling. Due to increasing circulating levels of Klotho the active agent abolished upregulation of collagen and downregulated of elastin in aortas leading to antennal of arterial remodeling in aged mice (FIGS. 4AAB). The active agent effectively decreased collagen production and increased elastin production (as shown in FIG. 4B), indicating that it is an effective antiaging treatment for providing arterial remodeling.

Aging increased arterial MMP activity and expression, which can be eliminated by the active agent. MMPs are a family of proteases that play important roles in extracellular matrix (ECM) remodeling and degradation increased. MMP activity contributes to ECM remodeling and fibrosis. MMPs activity was measured by zylography, MMP2 and MMP9 activities were increased. significantly in aortas of aged mice (FIG. 5A). Increasing circulating levels of Klotho by the active agent decreased MMP2 and MMP9 activities to the control levels of adult mice (FIG. 5A). MMPs expression levels were measured by western blot MMP2 and MMP9 protein expressions were increased significantly in aortas of aged mice (F1(J. 5B), Increasing circulating levels of Klotho by the active agent decreased MMP2 and MMP9 expressions to the control levels (FIG. 5B).

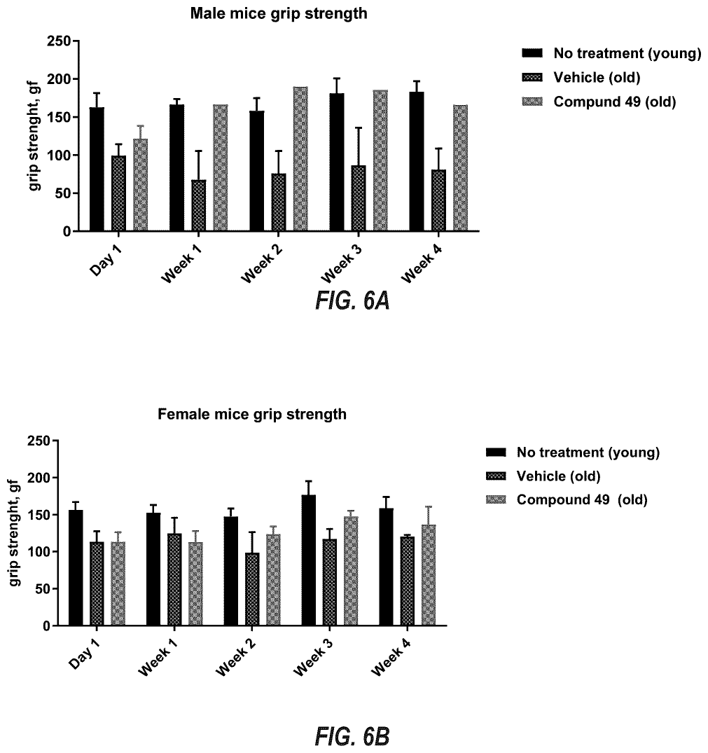

Aging increased arterial TGF-P1, TGF-P3, RUNX2 and ALP expression, which can be abolished by the active agent. TGFP increases matrix protein synthesis and decreases matrix protein degradation, resulting in tissue fibrosis Runt-related transcription factor 2 (RUNX2) and Alkaline phosphatase (ALP) are another two markers of fibrosis and arterial stiffening. Western blot analysis showed that TGF-1, TGF-P3, RUNX2 and ALP expressions were increased significantly in the aortas of aged mice (FIG. 6A-E). Increasing circulating levels of Klotho by the active agent decreased TGF˜t , TGF-18 3, RUNX2 and ALP expressions to the control levels of adult mice (FIG. 6A-E), indicating that aging-induced upregulation of TGF-1, TGF-P31 RUNX2 and ALP can be abolished by increasing circulating levels of Klotho.

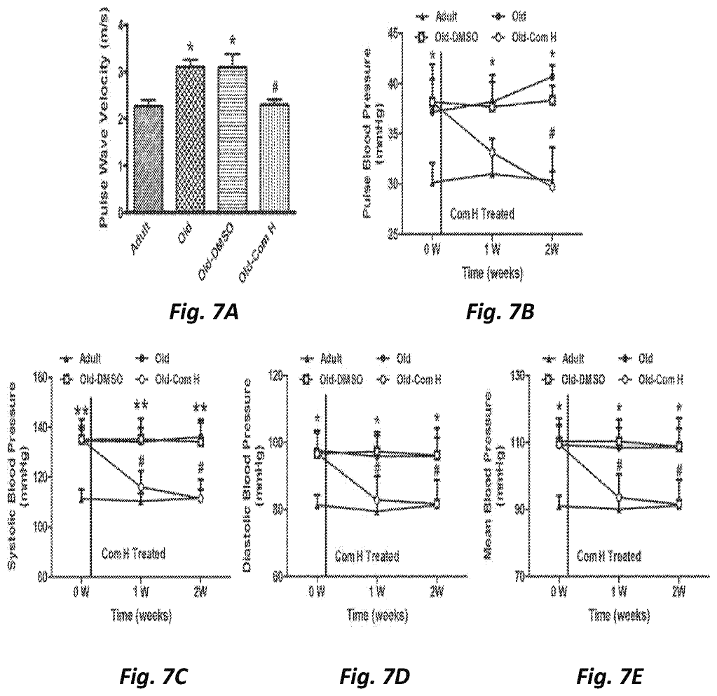

Aging inhibited SIRT1-A-1PK-eNOS pathway which can be activated by the active agent Silent information regulator T1 (SIRT1) plays an important role in the regulation of aging and longevity in mammals. The cross-talks between SIRT1 and AMP-activated protein kinase (AMPK), both activity, NOS activity, is indicated for controlling the senescence program, therefore, the SIRT1A-PK-eNOS cascade changing in aged mice and the effect of the active agent on this cascade was measured. The SIRT1 expression and activity in the aortas of aged mice were decreased significantly compared to adult mice. A.I though it did not affect S1RT1 expression, the active agent inquired SIRT1 activity significantly, as evidenced by an increase in deacetylation of p53 tumor suppressor protein (FIG. 7A). The phospho-AMPK and phosphoeNOS expression were also decreased in the aortas of aged mice, indicated AMPK and eNOS activity were decreased. However, the active agent increased AMPK and eNOS activity to the aortas of aged mice (FIGS. 7B and 7C). Therefore the SIRT1 -AMPK-eNOS cascade pathway was suppressed in the aortas of aged mice, which can be activated by the active agent.

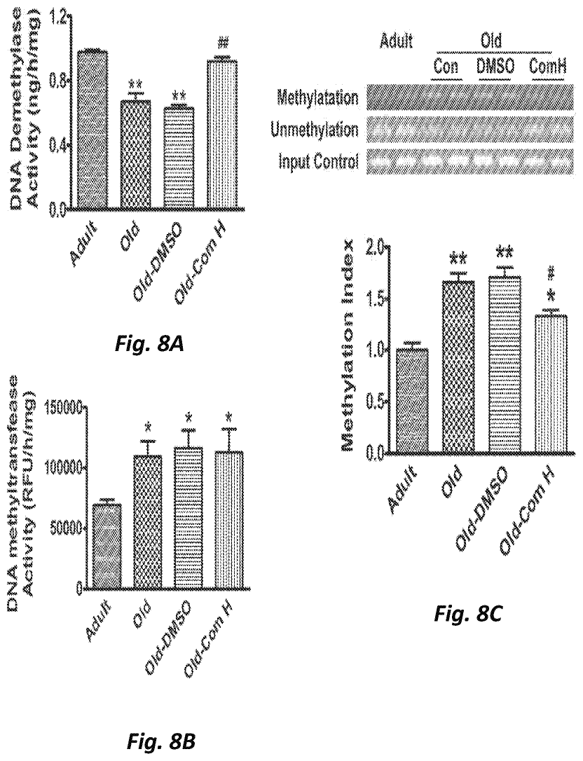

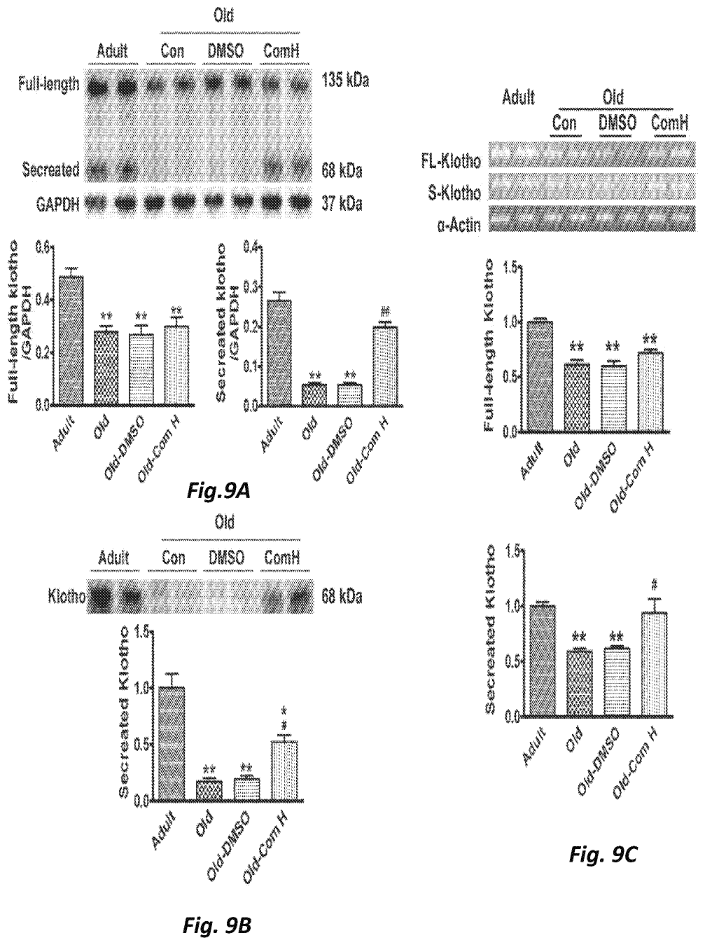

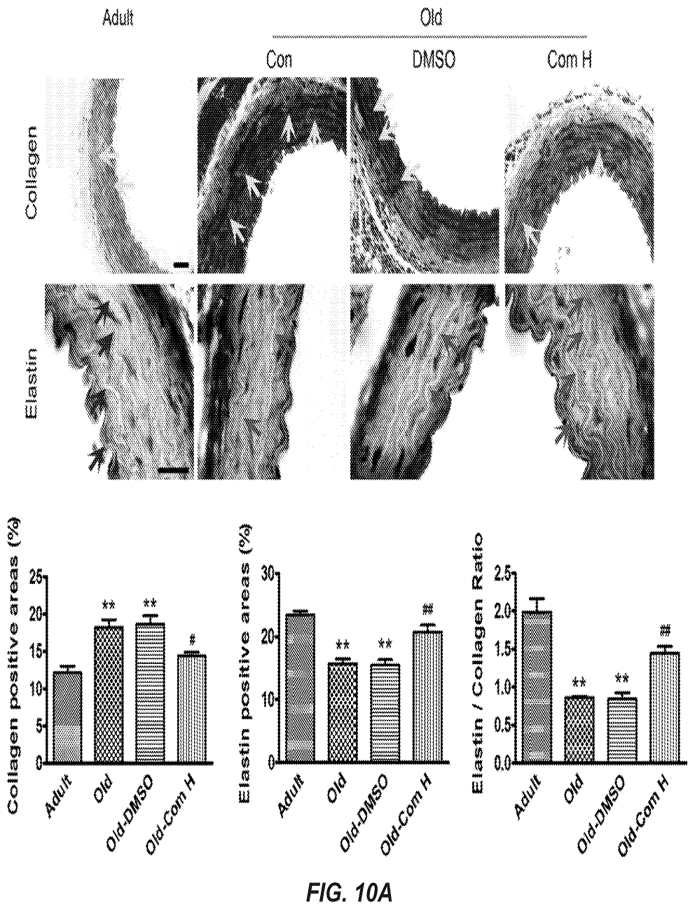

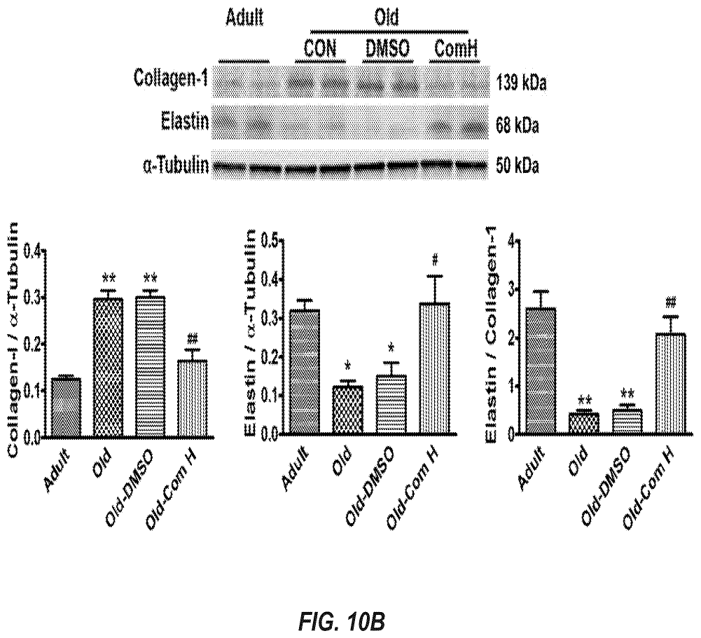

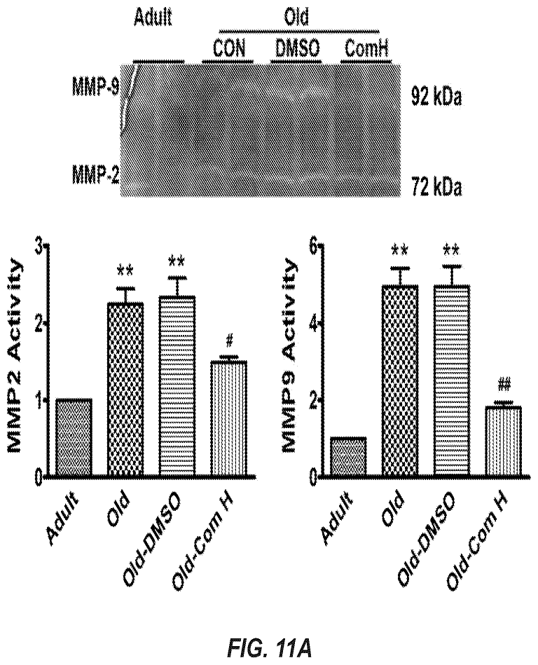

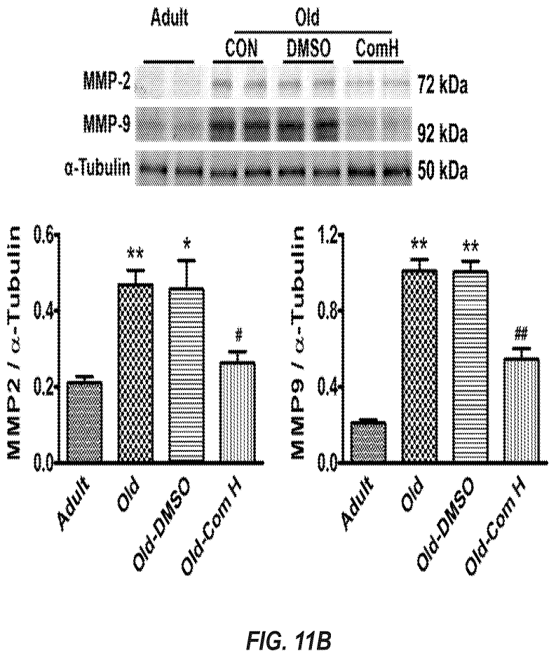

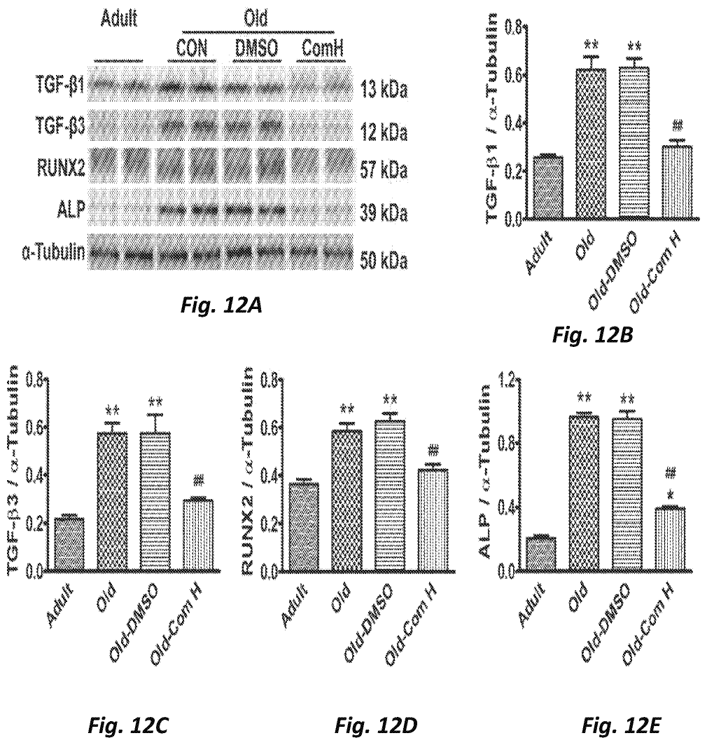

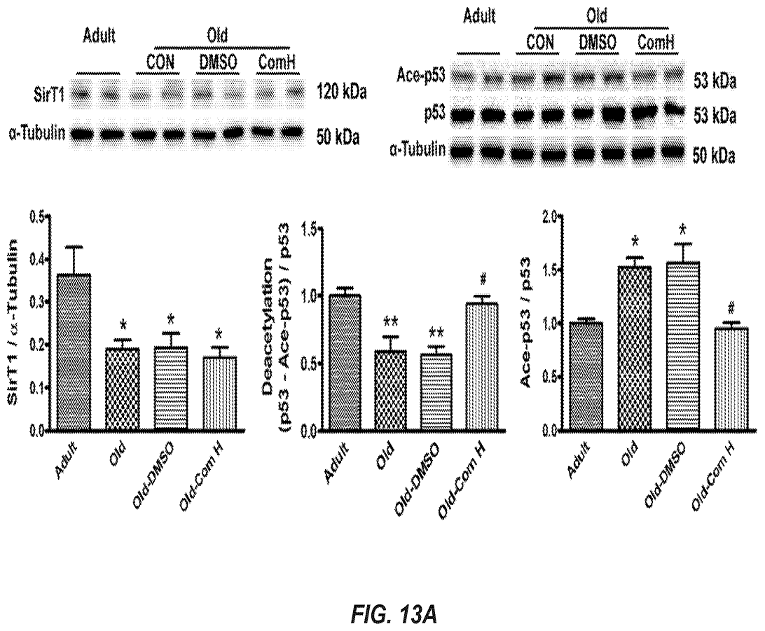

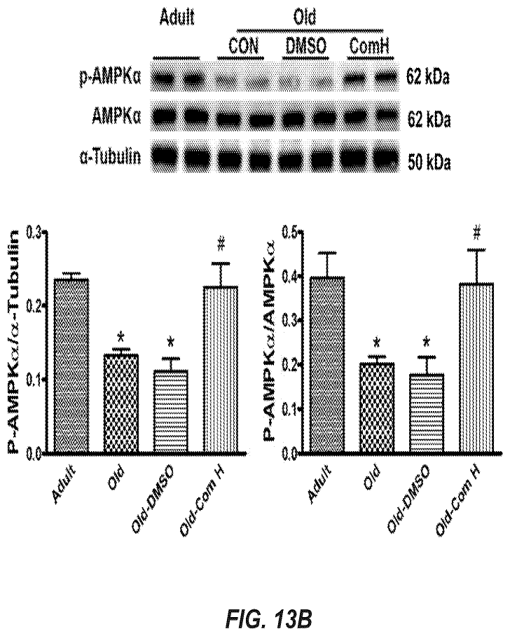

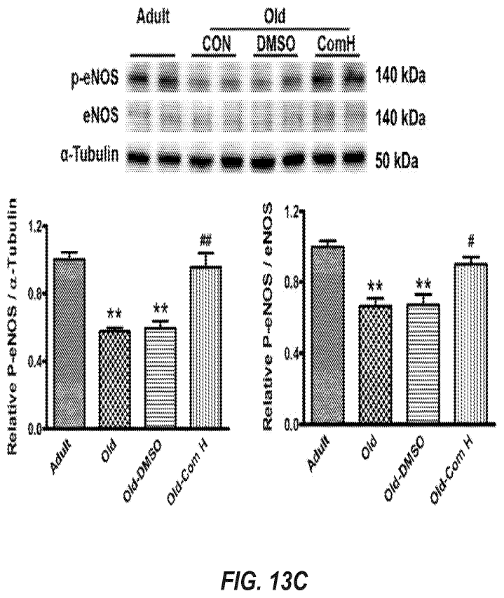

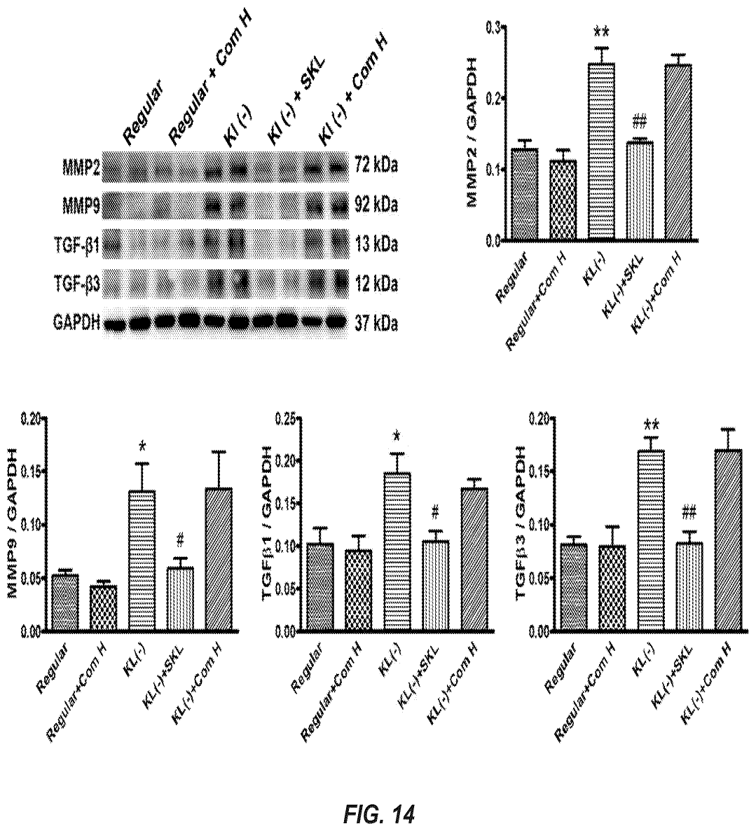

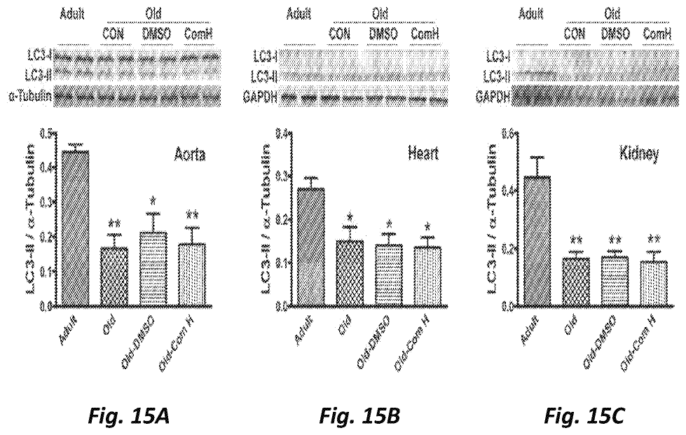

The active agent did not affect MMP2, MMP9, TGFP1, and TGFP3 expression in mouse vascular aortic smooth muscle cells (MO VAS), The in vivo example showed that the active agent increased dedicating levels of Klotho and attenuated arterial stiffening and hypertention of aged twice. Without wishing to be bond by theory, the results demonstrate that the active agent attenuates arterial stiffening and hypertension through increasing circulating levels of Klotho. Mouse aortic smooth muscle cells (MOVAS) which do not express endogenous Klotho were treated with active agent for 16 band then harvested for western blot analysis. In regular medium, which has constant levels of Klotho in the serum, active agent treatment did not change MMP2, MMP9, TGFP1 , and TGFP3 expressions (FIG. 8), indicating that active agent did not have direct effect on MMP2, MMP9, TGFP1, and TGFP3 expressions in smooth muscle cells. In Klotho free meilium (KL(−)) treated cells, MMP2, MMP9, TGFfH, and TGF-3 expressions were increased significantly compared to that of regular medium treated cells (FIG. 8), indicating that Klotho deficiency directly upregulates MMP2, MMP9, TGF-31, and TGF-3 expressions. However, secreted Klotho (sKL) treatment significantly decreased MMP2, MMP9, TGF˜1, and TGFP3 expressions (FIG. 8), indicating that Klotho directly regulates MMP2, MMP1, TGF-1, and TGFf33 expressions. But the active agent treatment did not affect MMP2, MMP9, TGFfH, and TGFP3 expressions in Klotho free medium treatment cells (FIG. 8). These results indicate that Klotho decreases MMP2. MMP9. TGflH, and TGP-3 expressions to inhibit arterial fibrosis and stiffness, and active agent has no direct effect on arterial fibrosis and stiffness without increasing Klotho expression. Therefore, the active agent attenuates arterial stiffening and hypertension through increasing circulating levels of Klotho. Another use of the active agent is an antiaging treatment for providing arterial remodeling which occurs as a result of a decrease in collagen production and an increase in elastin production (as shown in FIG. 48).

### Discussion

Aging is defined as the age-related decline in physiological function essential for survival fertility. Age can result in cardiovascular outcomes; such as arterial stiffness and hypertension. The results from the above described example, show that blood pressure (BP) and pulse wave velocity (PWV) were increased in 24-27 month old mice, with the age of equivalent of 70-80 years in humans. This demonstrates that activation of demethylase by the active agent reversed arterial stiffening and hypertension in aged mice (FIG. 1), indicating that increased methylation contributes to aging-refuted arterial stiffening and hypertension, and demonstrating that the active agent is an effective therapeutic agent for arterial stiffness and hypertension. Another part of the active agent is as an anti-aging treatment for providing arterial remodeling which occurs as a result of a decrease in collagen production and an increase in elevation production (as shown in FIG. 4B).

DNA methylation is one of several epigenetic mechanisms that cells use to control gene expression. During development, the pattern of DNA methylation in the genome changes as a result of a dynamic process involving both DNA methylation, and demethylation. The methylation state of the promoter region is related to Klotho mRNA expression, indicating that Klotho expression is regulated by DNA methylation. in above described example shows that DNA demethylase activity was decreased and the DNA methyltransferase activity was increased in aged mice (FIG. 2), indicating that aging increases DNA methylation.

DNA methylation of Klotho gene was increased in aged mice compared to that of adult mice, which was decreased by the active agent (FIG. 2). The active agent increased the DNA demethylase activity which was decreased in aged mice, while it did not affect the DNA methyltransferase activity. The active agent increased Klotho expression in aged mice through increasing the DNA demethylase activity to decrease the hypermethylation of Klotho gene. Therefore, the attenuation of aging-related arterial stiffening and hypertension by the active agent is attributed, at least in part, to increased Klotho levels. The example described herein shows that deficiency of Klotho gene caused arterial stiffness and hypertension. As discussed above, at age 70 years, the serum level of Klotho was a human is only about one half of what it was at age 40 years, while the prevalence of arterial stiffness and hypertension increases with age. Thus, Klotho deficiency is a pathological factor for aging associated arterial stiffness and hypertension.

Three types of Klotho protein with potentially different functions have been identified: (1) a full-length transmembrane Klotho; (2) a truncated soluble Klotho; and (3) a secreted Klotho. The full-length Klotho is mainly expressed in kidney distal tubule cells and serves as co-receptor of FIG. 23 and enhances FIG. 23 signaling to maintain mineral metabolism. While the circulating Klotho, inducing soluble Klotho and secreted Klotho, may act as a hormone and regulate the functions in tissues or cells that do not express Klotho (e.g., vascular endothelial cells and smooth muscle cells). In this example, both full-length and secreted Klotho were decreased in aged mouse, The active agent increased secreted Klotho but not the transmembrane form of Klotho.

The in vivo example shows that the active agent increased circulating levels of Klotho and attenuated arterial stiffening and hypertension in a genetic fodk-tin.g thaJaetive agent attenuated arterial stiffening and hypertension through increasing circulating levels of Mouse aortic smooth muscle cells (M′.OVAS) which do not express endogenous Klotho were treated with active agent and some arterial stiffness related protein expression was measured by western blot analysis. Because of no endogenous Klotho expression in MO VAS, the active agent could not affect MMP2, MMP9, TGFfI, and TGFfP3 expressions in either Klotho-contained medium or Klotho free medium. However, Klotho itself affects MMP2, MMP9, TGFf>1, and TGFP3expressions. Without wishing to be bound by theory, these results indicate that Klotho decreases MMP2, MMP9, TGFPI, and TGFP3 expressions to inhibit arterial fibrosis and stiffness) and that the active agent has no direct effect on anodal fibrosis and stiffness without increasing Klotho expression. Based on the results of the in vivo and in vitro examples discussed above, the active agent attenuates material stiffening and hypertension through increasing circulating levels of Klotho. Another use of the active agent is an antiaging treatment for providing arterial remodeling which occurs as a result of a decrease in collagen production and an increase in elastin. Production (as shown in FIG. 48),

The results of the example show that SIRT1 activity was increased in aged mice while the active agent increased SIRT1 activity. SIRT1, known as class II histone deacetylases is a nuclear protein implicated in the regulation: of many cellular processes, including apoptosis, cellular senescence, endocrine signaling, glucose homeostasis, aging, and longevity. SIRT1 has been reported to deacetylate the lysine residues of a number of nuclear proteins, such as p53, NFKB, PG1a. CBPip:300, and forehead family proteins. SIRT1 can inhibit TGF-P signaling and ameliorate fibrosis. In this example, aging decreased SIRT1 activity, increased TGFP and MMP expression, and induced fibrosis. So the inhibition of SIRT1 activity is the initial cause of aging induced fibrosis.

Exposure to the active agent decreased blood pressure after only 1 week or treatment. This acute effect is due to endothelium protection. Western blot results showed that the expression of pcNOS and pAMPK is decreased in aged mice, while the active agent increased their expression. Endothelial nitric-oxide symbols (eNOS) is an important enzyme in the cardiovascular system. It catalyzes the production of nitric oxide (NO), a key generator of blood pressure, vascular remodeling, and angiogenesis. Several protein kinases including AMPKB, PK.A, and AMPK activate eNOS by phosphorylating cell and in response to various stimuli. Due to increasing pAMPK and peNOS expressing, the active agent induce more NO production to protect endothelial function to low blood pressure.

While the present disclosure has been described herein in connection with certain embodiments so that aspects thereof may be more folly understood and appreciated, it is not intended that the present disclosure be limited to these particular embodiments. On the contrary, it is intended that all alternatives, modifications and equivalents are included within the scope of the present disclosure as defined herein. Titus the examples described above, which include particular embodiments, will serve to illustrate the practice of the inventive concepts of the present disclosure, it being understood that the particulars shown are by way of example and for purposes of instructive discussion of particular embodiments only and are presented for the cause of providing what is believed to be the most useful and readily understood description of procedures as well as of the principles and conceptual aspects of the present disclosure. Changes may be made in the formulation of the various compositions described herein, the methods described herein or in the steps or the sequence of steps of the methods described herein without departing from the spirit and scope of the present disclosure. Further, while various embodiments of the present disclosure have been described in claims herein below, it is not intended that the present disclosure be limited to these particular claims.

### References

The following references, to the extent that they provide exemplary procedural or other details supplementary to those set forth herein, are specifically incorporated herein by reference.

Avrahami D, Li Changhong, Schug, Avrahami Ran S, Stadler M B, Burger L, Schubeler D, Glaser B, Kaestner K H, Aging-Dependent Demethylation of Regulatory Elements Correlates with Chromatin State and Improved beta Cell Function. Cell Metab. 2015; 22 (4): 619.632.

Wagner W., Fernandez-Rebollo E, Frobel J, DNA-methylation changes in replicative senescence and aging: two sides of the same coin Epigenomics 2016- 8(1): 1-3.

Zinovkina LA, Zinovkin RA. DNA Metbylation, Mitochondria, and Programmed. Aging. Biochemistry (Mose). 20 15- 80 (12): 1571 -1577.

Bigot A, Duddy W J, Ouandaogo Z G, Negroni E, Mariot V, Ghimbovschi S, Harmon B, Wielgosik A, Loiseau C, Devaney J, Dumonceaux J, Butler-Browne G, Mouly V, Duguez S Age-Associated Methylation Suppresses SPRY1, Leading to a Failure of Re-quiescence and Loss of the Reserve Stem Cell Pool in Elderly Muscle. Cell Rep. 2015; 5; 13 (6): 1172- 1182.

Alvares S M, Mayberry G A, Joyner E Y, Lakowski B, Ahmed S. H3K4 demethylase activities repress proliferative and postmitotic aging. Aging Cell. 2014; 13 (2): 245-253.

Sun Z. Aging, arterial stiffness, and hypertension. Hypertension. 2015; 65 (2): 252-256,

Hashimoto J, Ito S. Central pulse pressure and aortic stiffness determine renal hemodynamics: pathophysiological implication for microalbuminuria in hypertension Hypertension. 2011; 58 (5): 839-846.

Hashimoto J. Ito S. Aortic stiffnes determines diastolic blood flow reversal in the descending thoracic aorta: potential implication for retrograde embolic stroke in hypertension. Hypertension. 2013; 62 (3): 542-549.

Karras A, Haymann J P, Bozec E, Metzger M, Jacquot C, Maruani G, Houillier P; Froissart Marc, Stengel B, Guardiola P, Laurent S, Boutouyrie P, Briet M, Nephro Test Study G. Large artery stiffening and remodeling are independently associated with all-cause mortality and cardiovascullar events in chronic kidney disease. Hypertension. 2012; 60 (6): 1451-1457.

Kitzman D W, Herrington D M, Brubaker P H, Moore J B, Eggeheen J, Haykowsky M J. Carotid arterial stiffness and its relationship to exercise intolerence in older patients with heart failure and preserved ejection fraction. Hypertension. 2013; 61 (1): 112-119.

Kuro-o M: Klotho in health and disease. Current opinion in Nephrology and hypertension, “2012”21 (4): 362-368.

Kuro-o M:, Matsumura Y, Aizawa H, Kawaguchi H, Suga T, Utsugi T, Ohyama Y, Kurabayash M, Kannme T, Kurne E, Iwasaki H, Iida A, Shiraki-Iida T, Nishikawa S, Nagai R, Nabeshima YI. Mutation of the mouse Klotho gene leads to a syndrome resembling ageing. Nature. 1997; 390 (6655): 45-51.

Kurosu H, Yamamoto M, Clark J D, Pastor J V, Nandi A, Gurnani P, McGuinness O P, Chikuda H, Yamaguchi M; Kawaguchi H; Shimomura I; Takayama Y˜Herz J, Kahn C R, Rosenblatt K P, Kuro-o M. Suppression of aging in mice by the hormone Klotho. Science. 2005; 309 (5742): 1829-1833.

Zhou L, Li Y, Zhou D, Tan R J, Liu Y. Loss of Klotho contributes to kidney injury by depression of Wnt/β-catenin signaling. Journal of the American Society of Nephrology: JASN. 2013; 24 (5): 771-785.

Lin Y˜Sun Z. In vivo pancreatic beta-cell-specific expression of antiaging gene Klotho: a novel approach for preserving beta-cells in type 2 diabetes. Diabetes, 2015; 64 (4): 1444-1458.

Doi S, Zou Y, Togao O, Pastor J V, John G B, Wang L, Shiizak K, Gotshall R, Schiavi S, Yorioka N, Takahashi M, Boothman D A, Kuro-o M. Klotho inhibits transforming growth factor-beta1 (TGF-beta1) signaling and suppresses renal fibrosis and cancer metastasis in mice. The Journal of biological chemistry. 201 1; 286 (10): 8655-8665.

Moe S M, Klotho: a master regulator of cardiovascular disease? Circulation. 2012; 125 (18): 2181-2183.

Chen K, Zhou X, Sun Z, Haplodeficiency of Klotho Gene Causes Arterial Stiffening via Upregulation of Scleraxis Expression and Induction of Autophagy. Hypertension. 2015; 66 (5): 1006N10.B.

Xiao N M, Zhang Y M, Zheng Q, Gu J. Klotho is a serum factor related to human aging. Chinese medical journal. 2004; 117 (5): 742-747.

Kotsis V, Stahouli S, Arterial stiffness, vascular aging, and intracranial large artery disease. American journal of hypertension. 2011; 24 (3) 252.

Karras A, Haymann J P, Bozec E, Metzger M, Jacquot C, Maruani G, Houillier P, Froissart M, Stengel B, Guardiola P, Laurent S, Boutouyrie P, Briet M. Large artery stiffening and remodeling are independently associated with all-cause mortality and cardiovascular events in chronic kidney disease. Hypertension. 2012; 60 (6): 1451-1457.

Hartley C J, Taffet G E, Michael L H, Pham T T, Entman M L. Noninvasive determination of pulse-wave velocity in mice. The American journal of physiology. 1997; 273 (I Pt 2): H494-500.

Steppan J, Barodka V, Berkowitz D E, Nyhan D. Vascular stiffness and increased pulse pressure in the aging cardiovascular system. Cardiology research and practice. 2011; 2011: 263585.

Capelli A, Lusuardi M, Cerutti C G, Donner C F. Lung alkaline phosphatase as a marker of fibrosis in chronic interstitial disorders, American journal of respiratory and critical care medicine. 1997˜155 (1): 249-253.

Lin M E, Chen T, Leaf E M, Speer M Y, Giachelli C M, Runx2 Expression in Smooth Muscle Cells Is Required for Arterial Medial Calcification in Mice, The American journal of pathology. 2015; 185 (7): 1958-1969,

Wang-F, Chen H Z, Lv X, Liu D P. SIRT1 is a novel potential treatment target for vascular aging and age-related vascular diseases. Current molecular medicine—2013; 13 (1): 155-164.

Satoh A, Brace C S, Rensing N, Cliften P, Wozniak D F, Herzog E D, Yamada K A, Imai S, SIRT1 extends life span and delays aging in mice through the regulation of Nk2 homeobox 1 in the DMH and LB. Cell metabolism. 2013; 18 (3): 416-430.

Alcendor R R, Gao S, Zhai P, Ziblocki D, Holle E, Yu X, Tian B, Wagner T, Vatner S F, Sadoshima J, SIRT1 regulates aging and resistance to oxidative stress in the heart, Circulation research, 2007; 100 (10): 1512-1521.

Wang Y, Liang Y, Vanhoutte P, SIRT1 and AMPK in regulating mammalian senescence: a critical review and a working model. FEBS letters. 2011;. 585 (7): 986-994,

Flurkey K, Currer J, Harrison D E. The Mouse in Aging Research. The Mouse in Biomedical. Research 2nd Edition, 2007: 637--672,

Moore L D, Le T, Fan G. DNA methylation and its basic function. Neuropsychopharmacology: official publication of the American College of Neuropsychopharmacology. 2013; 38 (1): 23-38.

Azuma M, Koyama D, Kikuchi J, Yoshizawa H, Thasinas D, Shiizaki K, Kuro-o M, Furukawa Y, Kusano E. Promoter methylation confers kidney-specific expression of the Klotho gene. FASEB journal: official publication of the federation of American Societies for Experimental Biology. 2012-26(10): 4264-4274.

King G D, Rosene D L, Abraham C R. Promoter methylation and age-related downregulation of Klotho in rhesus monkey. Age. 2012; 34 (6): 1405-1419.

Sun C Y, Chang S C, Wu M S. Suppression of Klotho expression by protein-bound uremic toxins is associated with increased DNA methyltransferase expression and DNA hypermethylation. Kidney international. 2012; 81 (7); 640-650.

Young G H, Wu V C, Klotho methylation is linked to uremic toxins and chronic kidney disease. Kidney international. 2012˜8.1 (7): 611 -612.

Chen K, Zhou X, Sun Z. Haplodeficiency of Klotho Gene Causes Arterial Stiffening via Upregulation of Scleraxis Expression and Induction of Autophagy. Hypertension. 2015.

Xu Y—Sun Z. Molecular basis of Klotho: from gene to function in aging. Endocrine reviews. 2015; 36 (2): 174-193.

Yuan F, Xie Q, Wu J, Bai Y, Mao B, Dong Y, Bi W, Ji G, Tao W, Wang Y, Yuan Z. MST1 promotes apoptosis through regulating Sirt1-dependent p53 deacetylation. The Journal of biological chemistry. 2011-286 (9); 6940-6945.

Salminen A, Kaarniranta K. NF-kappaB signaling in the aging process. Journal of clinical immunology. 2009; 29 (4): 397-405.

Amat R, Planavila A, Chen S L, Iglesias R., Giralt M, Villarroya F. SIRT1 controls the transcription of the peroxisome proliferator-activated receptor-gamma Co-activator-1alpha (PGC-1 alpha) gene in skeletal muscle through the PGC-1alpha autoregulatory loop and interaction with MyoD. The Journal of biological chemistry. 2009; 284 (33): 21872-21880,

Das C, Lucia M S, Hansen K C, Tyler J K, CBP/p300-mediated acetylation of histone H3 on lysine 56 Nature. 2009; 459 (7243): 113-117.

Brunet A, Sweeney L B, Sturgill J F, Chua K F, Greer P L, Lin Y, Tran H, Ross S E, Mostoslaysky R, Cohen H Y, Hu L S, Cheng H L, Jedrychowski M P, Gygi S P, Sinclair D A, Alt F W, Greenberg M E. Stress dependent regulation of FOXO transcription factors by the SIRT1 deacetylase. Science. 2004; 303 (5666): 2011-2015.

Kume S, Haneda M, Kanasaki K, Sugimoto T, Araki S, Isshiki K, Isono M, Uzu T, Guarente L, Kashiwagi A, Koya D. SIRT1 inhibits transforming growth factor beta-induced apoptosis in glomerular mesangial cells via Smad7 deacetylation. The Journal of biological chemistry. 2007; 282 (1): 151-158.

Zerr P, Palumbo-Zerr K, Huang J, Tomcik M, Sumova B, Distler O, Schett G, Disler J H, SIRT1 regulates canonical TGF-beta signaling to control fibroblast activation and tissue fibrosis. Annals of the rheumatic diseases. 2014.

Huang X Z, Wen D, Zhang M, Xie Q, Ma L, Guan Y, Ren Y, Chen J, Hao C M. Sirt1 activation ameliorates renal fibrosis by inhibiting the TGF-beta/Smad.3 pathway. Journal of cell biochemistry. 2014; 115 (5): 996-1005.

Shaul P W, Regulation of endothelial nitric oxide synthase location, location, location. Annual review of physiology. 2002; 64: 749-774.

Dimmeler S, Fleming I, Fisslthaler B, Hermann C, Busse R, Zeiher A M. Activation of nitric oxide synthase in endothelial cells by Akt-dependent physphorylation. Nature. 1999; 399 (6736): 601-605.

Fulton D, Gratton J P, McCabe T J, Fontana J, Fujio Y, Walsh K, Franke T F, Papapetropoulos A, Sessa W C. Regulation of endothelium-derived nitric oxide production by the protein kinase Akt. Nature. 1999˜199 (6736): 597-601,

Hartley C J, Taffet G E, Michael L H, Pham T T, Entman M L. Noninvasive determination of pulse-wave velocity in mice. The American journal of physiology. 1997; 273: H494-500.

Reddy A K, Li Y H, Pham T T, Ochoa L N˜Trevino M T, Hartley C J, Michael L H, Entman M L. Taffet G E. Measurement of aortic input impedance in mice: Effects of age on aortic stiffness. American journal of physiology. Heart and circulatory physiology. 2003; 285:8 1464-1470.

Whitesall S E, Hoff J B, Vollmer A P, D'Alecy L G. Comparison of simultaneous measurement of mouse systolic arterial blood pressure by radiotelemetry and tail-cuff methods. Am J. Physiol Heart Circ Physiol. 2004; 286: H2408M2415.

Feng M, Whitesall S, Zhang Y, Beibel M, D'Alecy L, DiPetrillo K. Validation of volume-pressure recording tail-cuff blood pressure measurements. Am J Hypertens. 2008; 21: 1288-1291.

Wang X, Skelley L, Wang B, Mejia A, Sapozhnikov V, Sun Z. AAV-based RNAi silencing of NADPH oxidase gp91(phox) attenuates cold-induced cardiovascular dysfunction. Hum. Gene Ther. 2012; .23: L016-1026,

Chen K, Kobayashi S, Xu X, Viollet B, Liang Q. Amp activated protein kinase is indispensable for myocardial adaptation to caloric restriction in mice. PloS one. 2013; 8: c59682.

Sun CY. Chang SC, Wu MS. Suppression of Klotho expression by protein-bound uremic toxins is associated with increased DNA methyltransferase expression and DNA hypermethylation Kidney international 2012; 81: 640-650.

What is claimed is:

1. A method of treating at least one of artificial stiffness, hypertension in a subject in need of such therapy, comprising:
administering to the subject an effective amount of a compound comprising an N-halophenyl-1H-indole-3-carboxamide, or a pharmaceutically-acceptable salt thereof, wherein the N-halophenyl- 1H-indole-3-carboxamide has a halogen on at least one of the 2, 3, 4, 5, and 6-carbon positions of the phenyl ring, wherein the halogen is selected from the group consisting of chlorine (Cl), fluorine (F), bromine (Br), and iodine (I).

2. The method of claim 1 wherein the N-halophenyl-1H-indole-3-carboxamide or pharmaceutically-acceptable salt thereof is represented by Formula I: 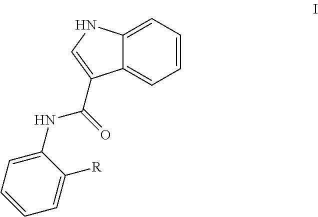
wherein R is selected from the group consisting of Cl, F, Br, and I.

3. The method of claim 2, wherein R is Cl.

4. A method of treating arterial aging in a subject in need of such therapy, comprising:
administering to the subject an effective amount of a compound that increases arterial elastin production, the compound comprising an N-halophenyl-1H-indole-3-carboxamide or a pharmaceutically-acceptable salt thereof, wherein the N-halophenyl-1H-indole-3-carboxamide has a halogen on at least one of the 2, 3, 4, 5, and 6-carbon positions of the phenyl ring, wherein the halogen is selected from the group consisting of chlorine (Cl), fluorine (F), bromine (Br). and iodine (1).

5. The method of claim 4, wherein the N-halophenyl-1H-indole-3-carboxamide or pharmaceutically-acceptable salt thereof is represented by Formula I: 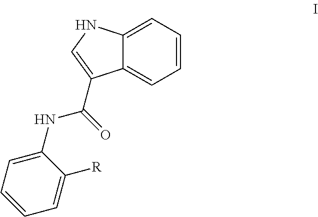
wherein R is selected from file group consisting of Cl, F, Br, and I.

6. The method of claim 5, wherein R is Cl.

7. The method of claim 4, wherein the compound decreases arterial collagen production.

8. A method of treating diabetes or a diabetes-related disease or condition in a subject in need of such therapy, comprising:
administering to the subject an effective amount of a compound comprising an halophenyl-1H-indole-3-carboxmide, or a pharmaceutically-acceptable salt thereof, wherein the N-halophenyl-1H-indole-3-carboxamide has a halogen on at least one of the 2, 3, 4, 5, and 6-carbon positions of the phenyl ring, wherein the halogen .is selected from the group consisting of chlorine (Cl), fluorine (F), bromine (Br), and iodine (I).

9. The method of claim 8, wherein the N-halophenyl-1H-indole-3-carboxamide or pharmaceutically-acceptable salt thereof is represented by Formula I: 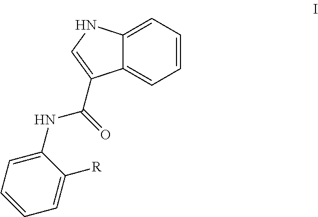
wherein R is selected from the group consisting of Cl, F, Br, and I,

10. The method of claim 9, wherein R is Cl.

11. The method of claim 8, wherein the diabetes or diabetes-related disease or condition is selected from the group consisting of Type 1 diabetes mellitus (T1DM), Type 2 diabetes mellitus (T2DM), hyperinsulinemia, obesity; peripheral arterial disease (PAD) of the arms, legs, and feet; foot ulcers; diabetic neuropathy; diabetic retinopathy; diabetic kidney disease; ketoacidosis; and
hyperosmolar hyperglycemic nonketotic syndrome (HHNS).

12. A method of (i) decreasing aortic expression of TGF-β1, TGF-β3, RUNX2, and ALP, (ii) increasing aortic expression of MMP2 and MMP9, (iii) increasing endothelial nitric oxide (NO) production, (iv) increasing kidney Klotho mRNA expression, (v) increasing serum Klotho protein levels, (vi) increasing arterial elastin levels, and (vii) decreasing arterial collagen production and arterial collagen levels in a mammalian subject, the method comprising:
administering to the mammalian subject an effective amount of a therapeutic composition, the effective amount comprising about 0.1 μg per kg of mammalian subject body weight to about 100 mg per kg of mammalian subject body weight, per dose, wherein administering the effective amount is sufficient to (i) decrease aortic expression of TGF-μ1, TGF-μ3, RUNX2, and ALP, (ii) increase aortic expression of MMP2 and MMP9, (iii) increase endothelial nitric oxide (NO) production, (iv) increase serum Klotho protein levels, (v) increase kidney Klotho mRNA expression, (vi) increase arterial elastin levels, and (vii) decrease arterial collagen production and arterial collagen levels in the mammalian subject, the therapeutic composition comprising:
an N-halophenyl-1H-indole-3-carboxamide as represented by Formula I, or a pharmaceutically-acceptable salt thereof: 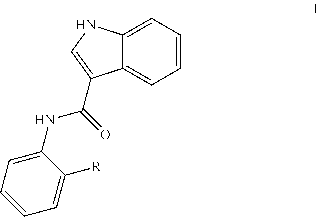
wherein R is selected from the group consisting of Cl, F, Br, and I; and a pharmaceutically acceptable carrier,
wherein administering the composition increases DNA demethylase activity and decreases methylation of the Klotho gene and (i) decreases aortic expression of TGF-β1, TGF-β3, RUNX2, and ALP, (ii) increases aortic expression of MMP2 and MMP9, (iii) increases endothelial nitric oxide (NO) production, (iv) increases serum Klotho protein levels, (v) increases kidney Klotho mRNA expression, (vi) increases arterial elastin levels, and (vii) decreases arterial collagen production and arterial collagen levels in the mammalian subject.

13. The method of claim 12, wherein R is Cl.
